(19) United States
(12) Patent Application Publication
(10) Pub. No.: US 2014/0088178 A1
(43) Pub. Date: Mar. 27, 2014

(54) COMBINATION OF ANTI-CLUSTERIN OLIGONUCLEOTIDE WITH ANDROGEN RECEPTOR ANTAGONIST FOR THE TREATMENT OF PROSTATE CANCER

(71) Applicants: Martin E. Gleave, Vancouver (CA); Amina Zoubeidi, West Vancouver (CA)

(72) Inventors: Martin E. Gleave, Vancouver (CA); Amina Zoubeidi, West Vancouver (CA)

(73) Assignee: THE UNIVERSITY OF BRITISH COLUMBIA, Vancouver, BC (CA)

(21) Appl. No.: 14/005,179

(22) PCT Filed: Mar. 14, 2012
(86) PCT No.: PCT/IB2012/000609

§ 371 Date: Nov. 20, 2013

Related U.S. Application Data
(60) Provisional application No. 61/452,583, filed on Mar. 14, 2011.
(60) Provisional application No. 61/453,309, filed on Mar. 16, 2011.
(60) Provisional application No. 61/453,885, filed on Mar. 17, 2011.
(60) Provisional application No. 61/493,336, filed on Jun. 3, 2011.

(51) Int. Cl. A61K 31/4166; A61K 31/7088

(52) U.S. Cl. CPC A61K 31/4166; A61K 31/7088
USPC 514/44 R

(57) ABSTRACT

A method for treating a mammalian subject afflicted with prostate cancer comprising i) an oligonucleotide which reduces clusterin expression and ii) an androgen receptor antagonist having the structure

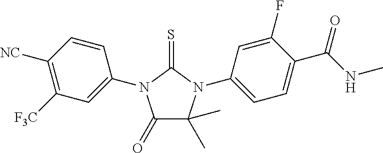 [SMILES: CNC(=O)c1ccc(N2C(=S)N(c3ccc(C#N)c(C(F)(F)F)c3)C(=O)C2(C)C)cc1F]

or a pharmaceutically acceptable salt thereof, each in an amount that when in combination with the other is effective to treat the mammalian subject.

This application claims priority of U.S. Provisional Application Nos. 61/452,583, filed Mar. 14, 2011, 61/453,309, filed Mar. 16, 2011, 61/453,885, filed Mar. 17, 2011, and 61/493,336, filed Jun. 3, 2011, the contents of which are hereby incorporated by reference.

Throughout this application, various publications are referenced, including referenced in parenthesis. Full citations for publications referenced in parenthesis may be found listed in alphabetical order at the end of the specification immediately preceding the claims. The disclosures of all referenced publications in their entireties are hereby incorporated by reference into this application in order to more fully describe the state of the art to which this invention pertains.

### FIELD OF THE INVENTION

The subject invention relates to combination therapy for treating prostate cancer.

### BACKGROUND OF THE INVENTION

Prostate cancer is the most common cancer that affects men, and the second leading cause of cancer deaths in men in the Western world. Because prostate cancer is an androgen sensitive tumor, androgen withdrawal, for example via castration, is utilized in some therapeutic regimens for patients with advanced prostate cancer. Androgen withdrawal leads to extensive apoptosis in the prostate tumor, and hence to a regression of the disease. However, castration-induced apoptosis is not complete, and a progression of surviving tumor cells to androgen-independence ultimately occurs. This progression is the main obstacle to improving survival and quality of life, and therapies capable of treating prostate cancer both before and after the progression to androgen independence are needed.

It has been observed that numerous proteins are expressed in increased amounts by prostate tumor cells following androgen withdrawal. At least some of these proteins are assumed to be associated with the apoptotic cell death which is observed upon androgen withdrawal. (Raffo et al., 1995; Krajewska et al., 1996; McDonnell et al., 1992). The functions of many of the proteins, however, is not completely understood. Clusterin (also known as sulfated glycoprotein-2 (SGP-2) or TRPM-2) is within this latter category.

### Clusterin

Clusterin is a cytoprotective chaperone protein that promotes cell survival and confers broad-spectrum resistance to cancer treatments (Chi et al. 2005). In Sensibar et al., Cancer Research 55: 2431-2437, 1995, the authors reported on LNCaP cells transfected with a gene encoding clusterin, and watched to see if expression of this protein altered the effects of tumor necrosis factor α (TNFα), to which LNCaP cells are very sensitive. Treatment of the transfected LNCaP cells with TNFα was shown to result in a transient increase in clusterin levels for a period of a few hours, but these levels had dissipated by the time DNA fragmentation preceding cell death was observed.

As described in U.S. Pat. No. 7,534,773, the contents of which are incorporated by reference, enhancement of castration-induced tumor cell death and delay of the progression of androgen-sensitive cancer cells to androgen-independence may be achieved by inhibiting the expression of clusterin by the cells.

### Custirsen

Custirsen is a second-generation antisense oligonucleotide that inhibits clusterin expression. Custirsen is designed specifically to bind to a portion of clusterin mRNA, resulting in the inhibition of the production of clusterin protein. The structure of custirsen is available, for example, in U.S. Pat. No. 6,900,187, the contents of which are incorporated herein by reference. A broad range of studies have shown that custirsen potently regulates the expression of clusterin, facilitates apoptosis, and sensitizes cancerous human prostate, breast, ovarian, lung, renal, bladder, and melanoma cells to chemotherapy (Miyake et al. 2005), see also, U.S. Patent Application Publication No. 2008/0119425 A1. In a clinical trial for androgen-dependent prostate cancer, the drugs flutamide and buserelin were used together in combination with custirsen, increasing prostate cancer cell apoptosis (Chi et al. 2004; Chi et al., 2005).

### Androgen Receptor Antagonists

Androgen receptor (AR) antagonists reduce the stimulation of prostate cancer cells by androgens by perturbing or reducing a function of AR, including androgen-AR binding, AR transcriptional activity, or cellular transport of AR such as translocation from the cytoplasm to the nucleus. Custirsen is not an AR antagonist. Custirsen inhibits the progression of prostate cancer to androgen independence by reducing the anti-apoptotic effects of clusterin and is not thought to affect androgen signaling pathways.

### Combination Therapy

The administration of two drugs to treat a given condition, such as prostate cancer, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry. In vivo drug metabolism/drug interaction studies—study design, data analysis, and recommendations for dosing and labeling). Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry. In vivo drug metabolism/drug interaction studies—study design, data analysis, and recommendations for dosing and labeling). The interaction may also heighten or lessen the side effects of each drug. Hence, upon administration of two drugs to treat a disease, it is unpredictable what change will occur in the profile of each drug.

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry. In vivo drug metabolism/drug interaction studies—study design, data analysis, and recommendations for dosing and labeling).

Thus, the success of one drug or each drug alone in an in vitro model, an animal model, or in humans, may not correlate into efficacy when both drugs are administered to humans.

### SUMMARY OF THE INVENTION

The present invention relates to a method for treating a mammalian subject afflicted with prostate cancer comprising administering to the mammalian subject i) an oligonucleotide which reduces clusterin expression and ii) an androgen receptor antagonist having the structure

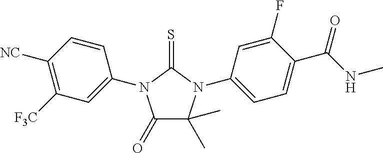 [SMILES: CNC(=O)c1ccc(N2C(=S)N(c3ccc(C#N)c(C(F)(F)F)c3)C(=O)C2(C)C)cc1F]

or a pharmaceutically acceptable salt thereof, each in an amount that when in combination with the other is effective to treat the mammalian subject.

Some embodiments of the invention provide a method for treatment of a mammalian subject afflicted with androgen-independent prostate cancer, consisting of administering to the subject i) an oligonucleotide which reduces clusterin expression, and ii) an androgen receptor antagonist, each in an amount that when in combination with the other is effective to treat the mammalian subject.

An aspect of the present invention provides a pharmaceutical composition comprising an amount of an oligonucleotide which reduces clusterin expression, and an androgen receptor antagonist for use in treating a mammalian subject afflicted with androgen-independent prostate cancer.

An aspect of the present invention provides an oligonucleotide which reduces clusterin expression for use in combination with an androgen receptor antagonist in treating a mammalian subject afflicted with androgen-independent prostate cancer.

An aspect of the present invention provides a composition for treating a mammalian subject afflicted with prostate cancer comprising i) an oligonucleotide which reduces clusterin expression and ii) an androgen receptor antagonist having the structure

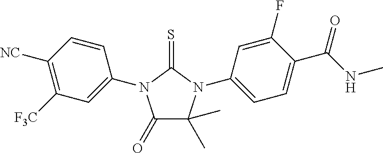 [SMILES: CNC(=O)c1ccc(N2C(=S)N(c3ccc(C#N)c(C(F)(F)F)c3)C(=O)C2(C)C)cc1F]

or a pharmaceutically acceptable salt thereof.

### BRIEF DESCRIPTION OF THE DRAWINGS

FIG. 1. Inhibition of LNCaP cell proliferation upon treatment with 1 μM AR1 and 10 nM siRNA targeting clusterin (CLU) or 10 nM SCR. SCR is a scrambled sequence siRNA control. (A), FBS condition is media supplemented with FBS. (B), CSS condition is charcoal serum stripped media.

FIG. 2. Inhibition of LNCaP cell proliferation upon treatment with 1 μM AR1 and 500 nM custirsen or 500 nM SCRB. SCRB is a scrambled sequence antisense oligonucleotide control. (A), FBS condition is media supplemented with FBS. (B), CSS condition is charcoal serum stripped media.

FIG. 3. Inhibition of C4-2 cell proliferation upon treatment with 1 μM AR1 and 500 nM custirsen or 500 nM SCBR. (A), FBS condition is media supplemented with FBS. (B), CSS condition is charcoal serum stripped media.

FIG. 4. PC-3 (AR-negative) cell proliferation upon treatment with 1 μM AR1 and 10 nM siRNA targeting clusterin or 10 nM SCR (A). PC-3 (AR-negative) cell proliferation upon treatment with 1 μM AR1 and 500 nM custirsen or 500 nM SCRB (B).

FIG. 5. Cytotoxicity in LNCaP cells upon treatment with AR1 and 10 nM siRNA targeting clusterin or 10 nM SCR (A). Cytotoxicity in LNCaP cells upon treatment with 1 μM AR1 and 500 nM custirsen or 500 nM SCRB (B). Cells grown in media supplemented with FBS. X-axis is AR1 concentration.

FIG. 6. Potency of AR1 and custirsen combination therapy in LNCaP cells. (A), Cell growth inhibition after treatment with each drug or a combination thereof by crystal violet assay. X-axis is [AR1]/[custirsen]. P-value was calculated by the Friedman test. (B), Dose effect curve for each treatment. (C), Combination index (CI) at several effective doses. CI=1, additive effect; CI<1, combination effect; CI>1, antagonistic effect.

FIG. 7. Cell Cycle Distribution upon treatment of AR1, siRNA targeting clusterin, or a combination thereof in LNCaP cells. OTR refers to cells treated with oligofectamine transfection reagent (Invitrogen Life Technologies, Inc.) in the absence of custirsen or siRNA.

FIG. 8. FACS Analysis of Cell Cycle Distribution upon treatment of AR1, custirsen, or a combination thereof in LNCaP cells.

FIG. 9. Effect of AR1 administration on AR and clusterin protein expression in LNCaP cells. (A), 10 μM AR1. (B), after 48 hours of AR1 treatment at indicated concentrations.

FIG. 10. Effect of AR1 administration on AKT and ERK phosphorylation and protein levels in LNCaP cells. (A), 10 μM AR1. (B), after 48 hours of AR1 treatment at indicated concentrations. (C), Dose dependent change of expression level of AKT or ERK after treatment with AR1. (D), Dose dependent change of expression level of AKT or ERK after treatment with AR1.

FIG. 11. Effect of AR1 administration on AR and clusterin mRNA expression in LNCaP cells. (A), AR mRNA expression 48 hours after adding AR1 at each concentration. (B), AR mRNA expression at indicated time points following the addition of 10 μM AR1. (C), clusterin mRNA expression 48 hours after adding AR1 at each concentration. (D), Clusterin mRNA expression at indicated time points following the addition of 10 μM AR1.

FIG. 12. Change of protein expression in LNCAP cells after treatment of siRNA targeting clusterin, custirsen, or indicated controls (A and C). (B), comparison of clusterin upregulation by bicalutamide and AR1. (D), Expression of AR co-chaperone by treatment with AR1 and custirsen (ASO).

FIG. 13. Effect of 10 μM AR1 and 10 nM siRNA targeting clusterin on PSA in LNCaP cells (A), or on AR mRNA expression (B).

FIG. 14. Effect of 10 μM AR1 and 500 nM custirsen combination therapy on AR (A), or PSA mRNA expression (B).

FIG. 15. Western blot analysis of protein expression and PARP cleavage after treatment of LNCaP cells with AR1 and 10 nM siRNA targeting clusterin. FBS condition is media supplemented with FBS. CSS condition is charcoal serum stripped media.

FIG. 16. Effect of AR1 and 10 μM siRNA targeting clusterin on protein levels upon androgen stimulation in LNCaP cells. R1881 is a potent androgen that is also known as metribolone.

FIG. 17. Western blot analysis of protein expression and PARP cleavage upon treatment of LNCaP cells with AR1 and custirsen or control. Cells (1×10⁶) were seeded in 10 cm dishes with RPMI medium containing 5% FBS. The next day, cells were transfected with 500 nM custirsen or control for 48 h. 10 μM AR1 was then added to the cells for 48 hours before harvesting for Western blot analysis. AR and PSA expression were highly repressed by custirsen and AR1 combination therapy.

FIG. 18. Western blot analysis of protein expression and phosphorylation upon treatment of LNCaP cells with AR1 and 10 nM siRNA targeting clusterin, or control. Phospho-AKT and phosph-ERK are activated by AR1 treatment; however, AR1 and Clu siRNA combination therapy reduces levels of phosphorylated AKT and ERK protein. Combination treatment represses the AKT-mTOR-p70S6K pathway more potently than monotherapy.

FIG. 19. Western blot analysis of AR proteasome degradation upon treatment of LNCaP cells with a combination of AR1 and custirsen or an siRNA targeting clusterin. MG132 is a proteasome inhibitor, and CHX is cycloheximide, an inhibitor of protein biosynthesis. AR protein degradation is potently increased by AR1 and custirsen combination therapy.

FIG. 20. Effect of AR1 and custirsen combination therapy on AR transcriptional activity. Dual luciferase assay; LNCaP cells were transfected for 2 days with 500 nM custirsen in CSS. AR1 (1 uM) or DMSO was then added with or without R1881 (1 nM) for 24 hours before harvesting for analysis.

FIG. 21. Increased inhibition of AR translocation from the cytoplasm to the nucleus upon combination of 10 nM siRNA targeting clusterin with 10 μM AR1 compared to monotherapy. LNCaP cells were used.

FIG. 22. Increased inhibition of AR translocation from the cytoplasm to the nucleus upon combination of 10 nM siRNA targeting clusterin with 10 μM AR1 compared to monotherapy. LNCaP cells were used.

FIG. 23. Increased association of AR with ubiquitin upon combination treatment of 10 μM AR1 and 10 nM siRNA targeting clusterin compared to monotherapy (A). Association of AR with ubiquitin upon combination treatment of 10 μM AR1 and 10 nM siRNA targeting clusterin, or control in the presence of MG132 (B). LNCaP cells were used.

FIG. 24. Comparison of clusterin knock-down between treatment of bicalutamide or AR1, in combination with custirsen (ASO) or control in LNCaP cells.

FIG. 25. Effect of (FKBP52) over-expression on AR degradation and clusterin knock-down in LNCaP cells.

FIG. 26. Decreased castration-resistant prostate cancer tumor growth and increased survival upon combination treatment of AR1 and custirsen in mice. Male athymic nude mice were injected s.c. in two sites with LNCaP cells in Matrigel. The mice were castrated once tumors reached 150 mm³ or the PSA level increased above 50 ng/mL. Once tumors progressed to castration resistance (PSA levels increased to the same level as pre-castration), 10 mice were randomly assigned to each of AR1+scrambled antisense oligonucleotide (SCRB) or AR1+custirsen. Custirsen (10 mg/kg/each dose) or SCRB (10 mg/kg/each dose) was injected i.p. once daily for the first week and then three times per week. AR1 (10 mg/kg/each dose) was administered orally once daily (morning) 7 days per week for 8 to 12 weeks.

FIG. 27. Increased survival upon combination treatment of AR1 and custirsen in mice. Male athymic nude mice were injected s.c. in two sites with LNCaP cells in Matrigel. The mice were castrated once tumors reached 150 mm³ or the PSA level increased above 50 ng/mL. Once tumors progressed to castration resistance (PSA levels increased to the same level as pre-castration), 10 mice were randomly assigned to each of AR1+scrambled antisense oligonucleotide (SCRB) or AR1+custirsen. Custirsen (10 mg/kg/each dose) or SCRB (10 mg/kg/each dose) was injected i.p. once daily for the first week and then three times per week. AR1 (10 mg/kg/each dose) was administered orally once daily (morning) 7 days per week for 8 to 12 weeks.

FIG. 28. Decreased PSA protein expression upon combination treatment of AR1 and custirsen in mice. Male athymic nude mice were injected s.c. in two sites with LNCaP cells in Matrigel. The mice were castrated once tumors reached 150 mm³ or the PSA level increased above 50 ng/mL. Once tumors progressed to castration resistance (PSA levels increased to the same level as pre-castration), 10 mice were randomly assigned to each of AR1+scrambled antisense oligonucleotide (SCRB) or AR1+custirsen. Custirsen (10 mg/kg/each dose) or SCRB (10 mg/kg/each dose) was injected i.p. once daily for the first week and then three times per week. AR1 (10 mg/kg/each dose) was administered orally once daily (morning) 7 days per week for 8 to 12 weeks.

FIG. 29. Decreased PSA protein expression upon combination treatment of AR1 and custirsen in mice. Male athymic nude mice were injected s.c. in two sites with LNCaP cells in Matrigel. The mice were castrated once tumors reached 150 mm³ or the PSA level increased above 50 ng/mL. Once tumors progressed to castration resistance (PSA levels increased to the same level as pre-castration), 10 mice were randomly assigned to each of AR1+scrambled antisense oligonucleotide (SCRB) or AR1+custirsen. Custirsen (10 mg/kg/each dose) or SCRB (10 mg/kg/each dose) was injected i.p. once daily for the first week and then three times per week. AR1 (10 mg/kg/each dose) was administered orally once daily (morning) 7 days per week for 8 to 12 weeks.

FIG. 30. Clusterin expression is induced in AR1 resistant tumors. (A) Increased clusterin expression following AR1 treatment. (B) Increased clusterin expression following AR1 treatment in AR1 resistant tumors. (C) Clusterin expression is up-regulated in a time and dose dependent manner after AR1 treatment, as determined by Western blot analysis.

FIG. 31. Combination treatment of custirsen and AR1 is more effective than custirsen or AR1 monotherapy in CRPC LNCaP xenografts. AR1 plus custirsen treatment decreased AR and PSA expression in CRPC xenografts.

FIG. 32. Clusterin knockdown decreases AR transcriptional activity and expression of AR-dependent genes. Transmembrane protease serine 2 (TMPRSS2) mRNA levels decreased following AR1 treatment, clusterin knockdown, and AR1 treatment plus clusterin knockdown.

FIG. 33. Clusterin knockdown decreases AR protein levels when combined with AR1. The possible interaction between heat shock protein 27 (Hsp27) and AR contributing to AR transcriptional activity, PSA expression, and cell survival is depicted.

FIG. 34. Clusterin knockdown decreases heat shock factor protein 1 (HSF-1) transcription activity and expression of heat shock proteins.

FIG. 35. Clusterin overexpression increases HSF-1 activity.

FIG. 36. Possible mechanism of action for AR1 treatment plus custirsen treatment in a tumor cell.

FIG. 37. Clusterin and autophagy may play a role in stress and cancer. Increased clusterin expression following endoplasmic reticulum (ER) stress, chemo-stress, and androgen deprivation is depicted.

FIG. 38. AR1 treatment induces autophagy in LNCaP cells.

FIG. 39. Treatment stressors induce clusterin which co-localizes with LC3B in aggresomes.

FIG. 40. ER stress-induced autophagy is inhibited by clusterin silencing.

FIG. 41. CLU is induced by AR1 and highly expressed in AR1 resistant cells. C, Dose and time dependent AR1 induction of CLU. D, CLU is also induced in AR1 resistant cells by AR knock down. AR ASO also induces CLU in several AR1 resistant MR49F cells. CLU is high in AR1 resistant cells, e.g. MR49F.

FIG. 42. Induction of stress response (ER, YB-1), as well as cross talk of pAKT and MAPK.

FIG. 45. Effect of combination treatment on AR transactivation and translocation. A, Custirsen combined with AR1 treatment decreases AR transactivation more than custirsen or AR1 monotherapy. LNCaP cells were transfected 500 nmol/L of custirsen or SCRB control for 2 consecutive days, and at day 2, transiently cotransfected with 1 μg of PSA-luciferase and Renilla-luciferase. On the next day, the cells were treated 10 μmol/L of AR1, then added 1 nmol/L of R1881 or vehicle for 24 h. Cells were harvested, and luciferase activity was determined. Columns represent means of at least three independent experiments done in triplicate. PSA activation was normalized Renilla-luciferase activity. B, Effect of AR translocation by combination treatment. 24 hours after transfection with 10 nmol/L of CLU siRNA or SCR siRNA control, LNCaP cells were treated with DMSO, 10 μmol/L of AR1 and 1 nmol/L of R1881 for 30 minutes and fixed in methanol/acetone for immunofluorescence staining with anti-AR antibody. Nuclei were stained with DAPI. AR1 inhibited AR translocation from the cytoplasm to the nucleus. CLU knockdown combined with AR1 shows increased effects of inhibition of AR translocation.

### DETAILED DESCRIPTION OF THE INVENTION

The present invention relates to a method for treating a mammalian subject afflicted with prostate cancer comprising administering to the mammalian subject i) an oligonucleotide which reduces clusterin expression and ii) an androgen receptor antagonist having the structure

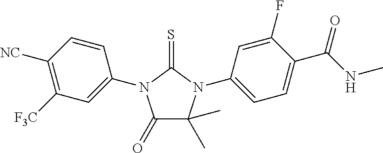 [SMILES: CNC(=O)c1ccc(N2C(=S)N(c3ccc(C#N)c(C(F)(F)F)c3)C(=O)C2(C)C)cc1F]

or a pharmaceutically acceptable salt thereof, each in an amount that when in combination with the other is effective to treat the mammalian subject.

In some embodiments of the invention the cancer is androgen-independent prostate cancer.

In some embodiments, the amount of the oligonucleotide and the amount of the androgen receptor antagonist or a pharmaceutically acceptable salt thereof when taken together is more effective to treat the subject than when each agent is administered alone.

In some embodiments, the amount of the oligonucleotide in combination with the amount of the androgen receptor antagonist or a pharmaceutically acceptable salt thereof is less than is clinically effective when administered alone.

In some embodiments, the amount of the androgen receptor antagonist or a pharmaceutically acceptable salt thereof in combination with the amount of the oligonucleotide is less than is clinically effective when administered alone.

In some embodiments, the amount of the oligonucleotide and the amount of the androgen receptor antagonist or a pharmaceutically acceptable salt thereof when taken together is effective to reduce a clinical symptom of prostate cancer in the subject.

In some embodiments, the mammalian subject is human.

In some embodiments, the oligonucleotide is an antisense oligonucleotide.

In some embodiments, the antisense oligonucleotide spans either the translation initiation site or the termination site of clusterin-encoding mRNA.

In some embodiments, the antisense oligonucleotide is modified to enhance in vivo stability relative to an unmodified oligonucleotide of the same sequence.

In some embodiments the antisense oligonucleotide consists essentially of an oligonucleotide selected from the group consisting of Seq. ID Nos. 1 to 11.

In some embodiments, the antisense oligonucleotide consists essentially of an oligonucleotide consisting of Seq. ID No. 3.

In some embodiments, the oligonucleotide is custirsen.

In some embodiments, the amount of custirsen is less than 640 mg.

In some embodiments, the amount of custirsen is less than 480 mg.

In some embodiments, the amount of custirsen is administered intravenously once in a seven day period.

In some embodiments, the amount of the androgen receptor antagonist is less than 240 mg.

In some embodiments, the amount of the androgen receptor antagonist is from 150 mg to 240 mg.

In some embodiments, the amount of the androgen receptor antagonist is from 30 mg to 150 mg.

In some embodiments, the amount of the androgen receptor antagonist is 80 mg.

In some embodiments, the amount of the androgen receptor antagonist is administered orally once per day.

Some embodiments of the invention provide a method for treatment of a mammalian subject afflicted with androgen-independent prostate cancer, consisting of administering to the subject i) an androgen receptor antagonist and ii) an oligonucleotide which reduces clusterin expression, each in an amount that when in combination with the other is effective to treat the mammalian subject.

In some embodiments the androgen receptor antagonist is a non-steroidal antiandrogen.

In some embodiments, the androgen receptor antagonist is AR1.

In some embodiments, the androgen-independent prostate cancer is resistant to AR1.

In some embodiments the combination of the androgen receptor antagonist and the oligonucleotide is effective to decrease androgen receptor translocation from the cytoplasm to the nucleus of the tumor cells.

In some embodiments, the combination of the androgen receptor antagonist and the oligonucleotide is effective to increase the proteasome degradation of the androgen receptor protein in the tumor cells.

In some embodiments, the combination of the androgen receptor antagonist and the oligonucleotide is effective to decrease androgen receptor transcriptional activity in the tumor cells.

In some embodiments, the combination of the androgen receptor antagonist and the oligonucleotide is effective to decrease the amount of phosphorylated AKT in the tumor cells.

In some embodiments, the combination of the androgen receptor antagonist and the oligonucleotide is effective to decrease the amount of phosphorylated ERK in the tumor cells.

In some embodiments, the combination of the androgen receptor antagonist and the oligonucleotide is effective to inhibit the proliferation of prostate cancer cells.

Some embodiments of the invention provide a method by which AR1 resistant prostate cancer cells are sensitized to AR1 by concomitant treatment with custirsen.

Some embodiments of the invention provide a method of increasing the sensitivity of AR1 resistant prostate cancer cells to AR1 comprising treating the AR1 resistant prostate cancer cells with custirsen.

Some embodiments of the invention provide a method for treatment of a mammalian subject afflicted with prostate cancer that is resistant to AR1, comprising administering to the subject i) AR1 and ii) custirsen, each in an amount that when in combination with the other is effective to treat the mammalian subject, wherein the custirsen increases the sensitivity of the prostate cancer to AR1.

An aspect of the present invention provides a pharmaceutical composition comprising an amount of an oligonucleotide which reduces clusterin expression, and an androgen receptor antagonist for use in treating a mammalian subject afflicted with androgen-independent prostate cancer.

An aspect of the present invention provides an oligonucleotide which reduces clusterin expression for use in combination with an androgen receptor antagonist in treating a mammalian subject afflicted with androgen-independent prostate cancer.

An aspect of the present invention provides a composition for treating a mammalian subject afflicted with prostate cancer comprising i) an oligonucleotide which reduces clusterin expression and ii) an androgen receptor antagonist having the structure

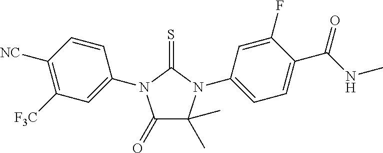 [SMILES: CNC(=O)c1ccc(N2C(=S)N(c3ccc(C#N)c(C(F)(F)F)c3)C(=O)C2(C)C)cc1F]

or a pharmaceutically acceptable salt thereof.

Aspects of the invention involve the increased potency of the combination of an oligonucleotide which decreases clusterin expression and an AR antagonist in the treatment of prostate cancer compared to oligonucleotide or AR antagonist monotherapy. Increased potency includes but is not limited to reduced proliferation of prostate cancer cells, increased apoptosis of cancer cells, reduced translocation of AR from the cytoplasm to the nucleus, reduced transcriptional activity of AR, increased PARP cleavage, reduced AKT phosphorylation, reduced ERK phosphorylation, and increased AR protein degradation. In embodiments in which AKT and/or ERK phosphorylation is reduced, all isoforms of AKT and ERK are envisioned. This includes but is not limited to AKT1, AKT2, AKT3, ERK1, and ERK2. In some embodiments, the increased proteasome degradation of AR involves the increased association of AR with ubiquitin.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” includes 0.2 mg/kg/day, 0.3 mg/kg/day, 0.4 mg/kg/day, 0.5 mg/kg/day, 0.6 mg/kg/day etc. up to 5.0 mg/kg/day.

### Terms

As used herein, and unless stated otherwise, each of the following terms shall have the definition set forth below.

As used herein, “about” in the context of a numerical value or range means±10% of the numerical value or range recited or claimed.

As used in the specification and claims of this application, the term “clusterin” refers to a glycoprotein present in mammals, including humans, and denominated as such in the humans. The sequences of numerous clusterin species are known. For example, the sequence of human clusterin is described by Wong et al., Eur. J. Biochem. 221 (3), 917-925 (1994), and in NCBI sequence accession number NM_{—}001831 (SEQ ID NO: 43). In this human sequence, the coding sequence spans bases 48 to 1397.

As used herein, “oligonucleotide which reduces clusterin expression” is an oligonucleotide with a sequence which is effective to reduce clusterin expression in a cell. The oligonucleotide which reduces clusterin expression may be, for example, an antisense oligonucleotide or an RNA interference inducing molecule.

As used herein, “antisense oligonucleotide” refers to a non-RNAi oligonucleotide that reduces clusterin expression and that has a sequence complementary to clusterin mRNA. Antisense oligonucleotides may be antisense oligodeoxynucleotides (ODN). Exemplary sequences which can be employed as antisense molecules in the invention are disclosed in PCT Patent Publication WO 00/49937, U.S. Patent Publication No. US 2002-0128220 A1, and U.S. Pat. No. 6,383,808, all of which are incorporated herein by reference. Specific antisense sequences are set forth in the present application as SEQ ID NOs: 1 to 18, and may be found in Table 1.

TABLE 1
Sequence Identification Numbers
for Antisense Oligonucleotides

| SEQ ID NO: | Sequence |
|---|---|
| 1 | GCACAGCAGG AGAATCTTCA T |
| 2 | TGGAGTCTTT GCACGCCTCG G |
| 3 | CAGCAGCAGA GTCTTCATCA T |
| 4 | ATTGTCTGAG ACCGTCTGGT C |
| 5 | CCTTCAGCTT TGTCTCTGAT T |
| 6 | AGCAGGGAGT CGATGCGGTC A |
| 7 | ATCAAGCTGC GGACGATGCG G |
| 8 | GCAGGCAGCC CGTGGAGTTG T |
| 9 | TTCAGCTGCT CCAGCAAGGA G |
| 10 | AATTTAGGGT TCTTCCTGGA G |
| 11 | GCTGGGCGGA GTTGGGGGCC T |
| 12 | GGTGTAGACG CCGCACG |
| 13 | GCAGCGCAGC CCCTGG |
| 14 | GCAGCAGCCG CAGCCCGGCT CC |
| 15 | AGCCGCAGCC CGGCTCCT |
| 16 | CAGCAGCCGC AGCCCGGCTC |
| 17 | CAGCAGCCGC AGCCCGGCTC |
| 18 | AGCAGCCGCA GCCCGGCTCC |

The ODNs employed may be modified to increase the stability of the ODN in vivo. For example, the ODNs may be employed as phosphorothioate derivatives (replacement of a non-bridging phosphoryl oxygen atom with a sulfur atom) which have increased resistance to nuclease digestion. MOE (2′-O-(2-methoxyethyl)) modification (ISIS backbone) is also effective. The construction of such modified ODNs is described in detail in U.S. Pat. No. 6,900,187 B2, the contents of which are incorporated by reference. In some embodiments, the ODN is custirsen.

As used herein, “custirsen” refers to an antisense oligonucleotide that reduces clusterin expression having the sequence CAGCAGCAGAGTCTTCATCAT (Seq. ID No.: 3), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. Custirsen is also known as TV-1011, OGX-011, ISIS 112989 and Custirsen Sodium.

As used herein, “RNA interference inducing molecule” refers to a molecule capable of inducing RNA interference or “RNAi” of clusterin expression. RNAi involves mRNA degradation, but many of the biochemical mechanisms underlying this interference are unknown. The use of RNAi has been described in Fire et al., 1998, Carthew et al., 2001, and Elbashir et al., 2001, the contents of which are incorporated herein by reference.

Isolated RNA molecules can mediate RNAi. That is, the isolated RNA molecules of the present invention mediate degradation or block expression of mRNA that is the transcriptional product of the gene, which is also referred to as a target gene. For convenience, such mRNA may also be referred to herein as mRNA to be degraded. The terms RNA, RNA molecule(s), RNA segment(s) and RNA fragment(s) may be used interchangeably to refer to RNA that mediates RNA interference. These terms include double-stranded RNA, small interfering RNA (siRNA), hairpin RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the RNA or internally (at one or more nucleotides of the RNA). Nucleotides in the RNA molecules of the present invention can also comprise nonstandard nucleotides, including non-naturally occurring nucleotides or deoxyribonucleotides. Collectively, all such altered RNAi molecules are referred to as analogs or analogs of naturally-occurring RNA. RNA of the present invention need only be sufficiently similar to natural RNA that it has the ability to mediate RNAi.

As used herein the phrase “mediate RNAi” refers to and indicates the ability to distinguish which mRNA molecules are to be afflicted with the RNAi machinery or process. RNA that mediates RNAi interacts with the RNAi machinery such that it directs the machinery to degrade particular mRNAs or to otherwise reduce the expression of the target protein. In one embodiment, the present invention relates to RNA molecules that direct cleavage of specific mRNA to which their sequence corresponds. It is not necessary that there be perfect correspondence of the sequences, but the correspondence must be sufficient to enable the RNA to direct RNAi inhibition by cleavage or blocking expression of the target mRNA.

As noted above, the RNA molecules of the present invention in general comprise an RNA portion and some additional portion, for example a deoxyribonucleotide portion. The total number of nucleotides in the RNA molecule is suitably less than in order to be effective mediators of RNAi. In preferred RNA molecules, the number of nucleotides is 16 to 29, more preferably 18 to 23, and most preferably 21-23. Suitable sequences are set forth in the present application as SEQ ID NOs: 19 to 42 (Table 2).

TABLE 2
Sequence Identification Numbers for
RNA Interference Inducing Molecules

| SEQ ID NO: | Sequence |
|---|---|
| 19 | CCAGAGCUCG CCCUUCUACTT |
| 20 | GUAGAAGGGC GAGCUCUGGTT |
| 21 | GAUGCUCAAC ACCUCCUCCTT |
| 22 | GGAGGAGGUG UUGAGCAUCTT |
| 23 | UAAUUCAACA AAACUGUTT |
| 24 | GACAGUUUUA UUGAAUUAGTT |
| 25 | UAAUUCAACA AAACUGUTT |
| 26 | ACAGUUUUGU UGAAUUATT |
| 27 | AUGAUGAAGA CUCUGCUGCTT |
| 28 | GCAGCAGAGU CUUCAUCAUTT |
| 29 | UGAAUGAAGG GACUAACCUG TT |
| 30 | CAGGUUAGUC CCUUCAUUCA TT |
| 31 | CAGAAAUAGA CAAAGUGGGG TT |
| 32 | CCCCACUUUG UCUAUUUCUG TT |
| 33 | ACAGAGACUA AGGGACCAGA TT |
| 34 | ACAGAGACUA AGGGACCAGA TT |
| 35 | CCAGAGCUCG CCCUUCUACT T |
| 36 | GUAGAAGGGC GAGCUCUGGT T |
| 37 | GUCCCGCAUC GUCCGCAGCT T |
| 38 | GCUGCGGACG AUGCGGGACT T |
| 39 | CUAAUUCAAU AAAACUGUCT T |
| 40 | GACAGUUUUA UUGAAUUAGT T |
| 41 | AUGAUGAAGA CUCUGCUGC |
| 42 | GCAGCAGAGU CUUCAUCAU |

The siRNA molecules of the invention are used in therapy to treat patients, including human patients, that have cancers or other diseases of a type where a therapeutic benefit is obtained by the inhibition of expression of the targeted protein. siRNA molecules of the invention are administered to patients by one or more daily injections (intravenous, subcutaneous or intrathecal) or by continuous intravenous or intrathecal administration for one or more treatment cycles to reach plasma and tissue concentrations suitable for the regulation of the targeted mRNA and protein.

As used herein, a “mammalian subject afflicted with prostate cancer” means a mammalian subject who was been affirmatively diagnosed to have prostate cancer.

As used herein, “androgen-independent prostate cancer” encompasses cells, and tumors predominantly containing cells, that are not androgen-dependent (not androgen sensitive). Often androgen-dependent cells progress from being androgen-dependent to being androgen-independent. Additionally, in some embodiments androgen-independent prostate cancer may encompass a tumor that overall is not androgen-dependent (not androgen sensitive) for growth. In some embodiments, androgen independent prostate cancer has progressed since the administration of hormone ablation therapy and/or the administration of an AR antagonist (as in hormone blockade therapy). In some embodiments, there is increased AR expression in the androgen-independent prostate cancer compared to prostate cancer that is not androgen-independent.

As used herein, “castration-resistant prostate cancer” encompasses any androgen-independent prostate cancer that is resistant to hormone ablation therapy or hormone blockade therapy. In some embodiments, castration-resistant prostate cancer has progressed since the administration of hormone ablation and/or hormone blockade therapy. In some embodiments, there is increased AR expression in the castration-resistant prostate cancer compared to prostate cancer that is not castration resistant.

As used herein, “androgen-withdrawal” encompasses a reduction in the level of an androgen in a patient afflicted with prostate cancer.

As used herein, “hormone blockade therapy” means a reduction in the function of receptors or cellular pathways that respond to an androgen. A non-limiting example of a hormone blockade therapy is an AR antagonist.

As used herein “androgen ablation therapy” is any therapy that is capable of causing androgen-withdrawal in a mammalian subject afflicted with prostate cancer. Terms used herein that are synonymous with androgen ablation therapy, are “androgen withdrawal” and “hormone ablation therapy”. Non-limiting examples of androgen ablation therapies include both surgical (removal of both testicals) and medical (drug induced suppression of testosterone or testosterone induced signaling) castration. Medical castration can be achieved by various regimens, including but not limited to LHRH agents, and agents that reduce androgen expression from a gland such as the adrenal glands (Gleave et al., 1999; Gleave et al., 1998).

As used herein, “AR antagonist” refers to an agent that perturbs or reduces a function of AR, including androgen binding, AR signaling, cellular transport of AR such as translocation from the cytoplasm to the nucleus, AR protein levels, or AR protein expression. AR antagonists include but are not limited to AR-specific monoclonal antibodies, oligonucleotides that target AR expression (such as AR-targeting antisense oligonucleotides or RNA inducing molecules), peptide agents specific for AR, and small molecule inhibitors specific for AR. An AR antagonist may be a non-steroidal antiandrogen such as AR1, bicalutamide, flutamide, nilutamide, RD162, and ZD4054.

AR1 is an AR antagonist of the invention having the structure:

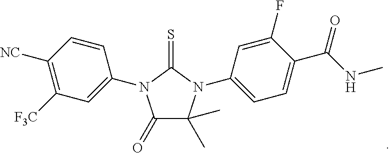 [SMILES: CNC(=O)c1ccc(N2C(=S)N(c3ccc(C#N)c(C(F)(F)F)c3)C(=O)C2(C)C)cc1F]

Methods of synthesis for AR1 are described in U.S. Pat. No. 7,709,517 B2, the contents of which are incorporated herein by reference. Alternatively, AR1 may be obtained from Medivation, Inc. (San Francisco, Calif., USA). The CAS Registry No. for AR1 is 915087-33-1, and its PubChem No. is 15951529. AR1 has the chemical formula C₂₁H₁₆F₄N₄O₂S, and is also known as MDV3100 and 4-(3-(4-cyano-3-(trifluoromethyl)phenyl)-5,5-dimethyl-4-oxo-2-thioxoimidazolidin-1-yl)-2-fluoro-N-methylbenzamide. AR1 is a second generation orally available AR antagonist that works by blocking androgen binding to AR, impeding the nuclear translocation of AR from the cytoplasm, and inhibiting AR-DNA binding (Tran et al. 2009). AR1 is currently being evaluated in clinical trials for the treatment of advanced prostate cancer (Scher et al., 2010).

As used herein, “transcriptional activity” refers to a protein's ability to bind or otherwise become directly or indirectly associated with a portion of DNA in a cell resulting in an influence on the level of expression of one or more genes.

The inhibition of clusterin expression may be transient, and may occur in combination with androgen ablation therapy or administration of an AR antagonist. In humans with prostate cancer that is not androgen-independent, this means that inhibition of expression should be effective starting within a day or two of androgen withdrawal or administration of an AR antagonist, and extending for about 3 to 6 months thereafter. This may require multiple doses to accomplish. It will be appreciated, however, that the period of time may be more prolonged, starting before castration and extending for substantial time afterwards without departing from the scope of the invention.

Aspects of the invention can be applied to the treatment of androgen-independent prostate cancer, or to prevent prostate cancer from becoming androgen-independent.

Aspects of the invention can be applied to the treatment of castration-resistant prostate cancer, or to prevent prostate cancer from becoming castration-resistant.

“Combination” means either at the same time and frequency, or more usually, at different times and frequencies than an oligonucleotide which reduces clusterin expression, as part of a single treatment plan. Aspects of the invention include the administration of the oligonucleotide before, after, and/or during the administration of an AR antagonist. An AR antagonist may therefore be used, in combination with the oligonucleotide according to the invention, but yet be administered at different times, different dosages, and at a different frequency, than a oligonucleotide which reduces clusterin expression.

As used herein, an “amount” or “dose” of an oligonucleotide measured in milligrams refers to the milligrams of oligonucleotide present in a drug product, regardless of the form of the drug product.

As used herein, “effective” when referring to an amount of oligonucleotide which reduces clusterin expression, an AR antagonist, or any combination thereof refers to the quantity of oligonucleotide, AR antagonist, or any combination thereof that is sufficient to yield a desired therapeutic response without undue adverse side effects (such as toxicity, irritation, or allergic response) commensurate with a reasonable benefit/risk ratio when used in the manner of this invention.

As used herein, “treating” encompasses, e.g., inhibition, regression, or stasis of the progression of prostate cancer. Treating also encompasses the prevention or amelioration of any symptom or symptoms of prostate cancer.

As used herein, “inhibition” of disease progression or disease complication in a subject means preventing or reducing the disease progression and/or disease complication in the subject.

As used herein, a “symptom” associated with prostate cancer includes any clinical or laboratory manifestation associated with prostate cancer, and is not limited to what the subject can feel or observe.

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds and/or combinations to the subject.

### Dosage Units

Administration of an oligonucleotide that targets clusterin expression can be carried out using the various mechanisms known in the art, including naked administration and administration in pharmaceutically acceptable lipid carriers. For example, lipid carriers for antisense delivery are disclosed in U.S. Pat. Nos. 5,855,911 and 5,417,978, which are incorporated herein by reference. In general, the oligonucleotide is administered by intravenous (i.v.), intraperitoneal (i.p.), subcutaneous (s.c.), or oral routes, or direct local tumor injection. In preferred embodiments, an oligonucleotide targeting clusterin expression is administered by i.v. injection. In some embodiments, the amount of oligonucleotide administered is 640 mg.

The amount of oligonucleotide administered is one effective to inhibit the expression of clusterin in prostate cells. It will be appreciated that this amount will vary both with the effectiveness of the oligonucleotide employed, and with the nature of any carrier used.

The amount of antisense oligonucleotide targeting clusterin expression administered may be from 40 to 640 mg, or 300-640 mg. Administration of the antisense oligonucleotide may be once in a seven day period, 3 times a week, or more specifically on days 1, 3 and 5, or 3, 5 and 7 of a seven day period. In some embodiments, administration of the antisense oligonucleotide is less frequent than once in a seven day period. Dosages may be calculated by patient weight, and therefore a dose range of about 1-20 mg/kg, or about 2-10 mg/kg, or about 3-7 mg/kg, or about 3-4 mg/kg could be used. This dosage is repeated at intervals as needed. One clinical concept is dosing once per week with 3 loading doses during week one of treatment. The amount of antisense oligonucleotide administered is one that has been demonstrated to be effective in human patients to inhibit the expression of clusterin in cancer cells.

In some embodiments of the invention, the amount of oligonucleotide targeting the expression of clusterin required for treatment of prostate cancer is less in combination with an AR antagonist, than would be required with oligonucleotide monotherapy.

Custirsen may be formulated at a concentration of 20 mg/mL as an isotonic, phosphate-buffered saline solution for IV administration and can be supplied as an 8 mL solution containing 160 mg custirsen sodium in a single vial.

Custirsen may be added to 250 mL 0.9% sodium chloride (normal saline). The dose may be administered using either a peripheral or central indwelling catheter intravenously as an infusion over 2 hours. Additionally, an infusion pump may be used.

Administration of an AR antagonist may be oral, nasal, pulmonary, parenteral, i.v., i.p., intra-articular, transdermal, intradermal, s.c., topical, intramuscular, rectal, intrathecal, intraocular, and buccal. A preferred route of administration for AR1 is oral. One of skill in the art will recognize that higher doses may be required for oral administration of an AR antagonist than for i.v. injection.

The dose of an AR antagonist may be 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 240 mg, 360 mg, 480 mg, or 600 mg. In some embodiments, the dose of an AR antagonist is less than 30 mg. In these embodiments the dose may be as low as 25 mg, 20 mg, 15 mg, 10 mg, 5 mg, or less. In some embodiments, the dose of an AR antagonist is administered daily. In some embodiments the dose is administered orally.

A dosage unit of the oligonucleotide which reduces clusterin expression and an AR antagonist may comprise one of each singly or mixtures thereof. A combination of an oligonucleotide which reduces clusterin expression and AR1 can be administered in oral dosage forms as tablets, capsules, pills, powders, granules, elixirs, tinctures, suspensions, syrups, and emulsions. An oligonucleotide which reduces clusterin expression and/or an AR antagonist may also be administered in intravenous (bolus or infusion), intraperitoneal, subcutaneous, or intramuscular form, or introduced directly, e.g. by injection or other methods, into or onto a prostate cancer lesion, all using dosage forms well known to those of ordinary skill in the pharmaceutical arts.

An oligonucleotide which reduces clusterin expression and/or an AR antagonist of the invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. An oligonucleotide which reduces clusterin expression and/or an AR antagonist can be administered alone or mixed with a pharmaceutically acceptable carrier. This carrier can be a solid or liquid, and the type of carrier is generally chosen based on the type of administration being used. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

An oligonucleotide which reduces clusterin expression and/or an AR antagonist can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

For oral administration in liquid dosage form, AR1 may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

In some embodiments of the invention, the amount of AR antagonist required for treatment of prostate cancer is less in combination with an oligonucleotide targeting the expression of clusterin, than would be required with AR antagonist monotherapy.

A dosage unit may comprise a single compound or mixtures of compounds. A dosage unit can be prepared for oral or injection dosage forms.

According to an aspect of the invention, there is provided an oligonucleotide which reduces clusterin expression-containing pharmaceutical composition packaged in dosage unit form, wherein the amount of the oligonucleotide in each dosage unit is 640 mg or less. Said pharmaceutical composition may include an AR antagonist, and may be in an injectable solution or suspension, which may further contain sodium ions.

According to another aspect of the invention, there is provided the use of an oligonucleotide targeting clusterin expression and an AR antagonist in the manufacture of a medicament for the treatment of cancer, where the medicament is formulated to deliver a dosage of 640 mg or less of oligonucleotide to a patient. The medicament may contain sodium ions, and/or be in the form of an injectable solution.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Clusterin Inhibitor Custirsen Together with AR Antagonist AR1 is a Potent Combination Therapy in Castration-Resistant Prostate Cancer Models

### Introduction and Objective

AR and intra-tumoral androgen synthesis are implicated in promoting tumor cell survival and development of castration-resistant prostate cancer (CRPC). AR1, has shown activity in preclinical and clinical studies. Previous studies link androgen ablation therapy with clusterin upregulation and castration resistance. The antisense inhibitor, custirsen, increases cell death when combined with castration or chemotherapy in prostate cancer (CaP) models. Herein below, the ability of custirsen and AR1 combination therapy to delay progression in a castration-resistant LNCaP model was tested.

### Methods

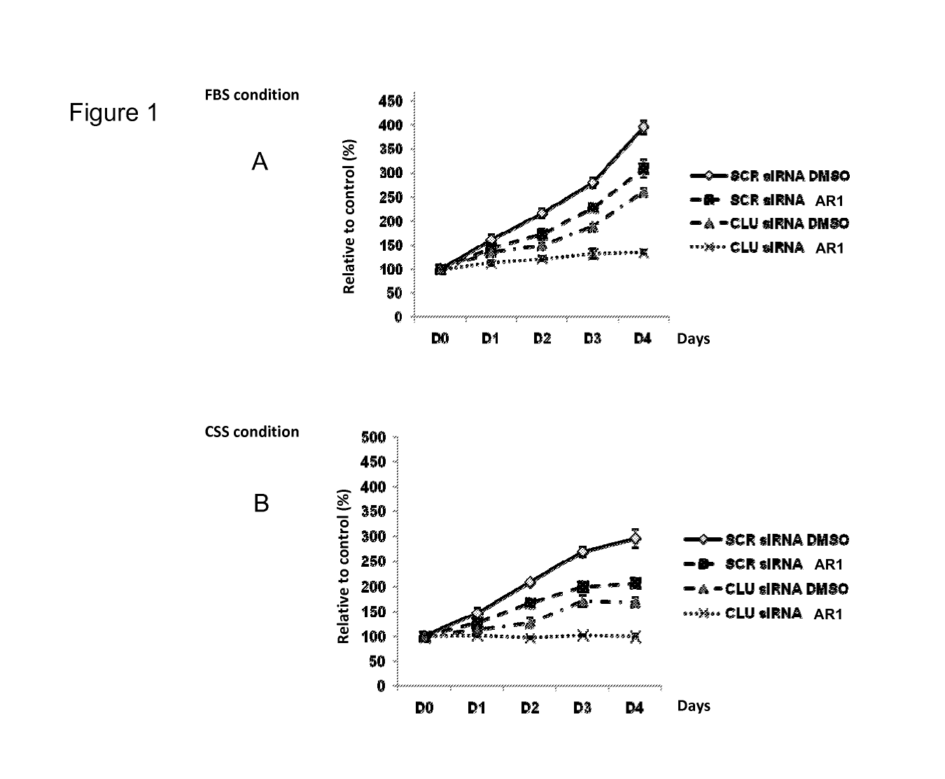

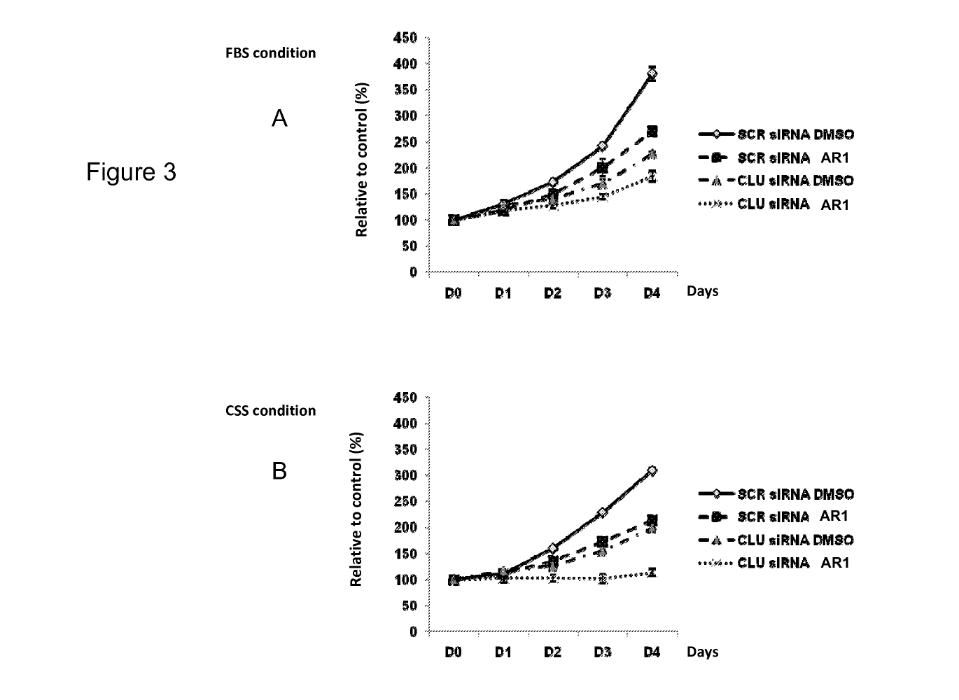

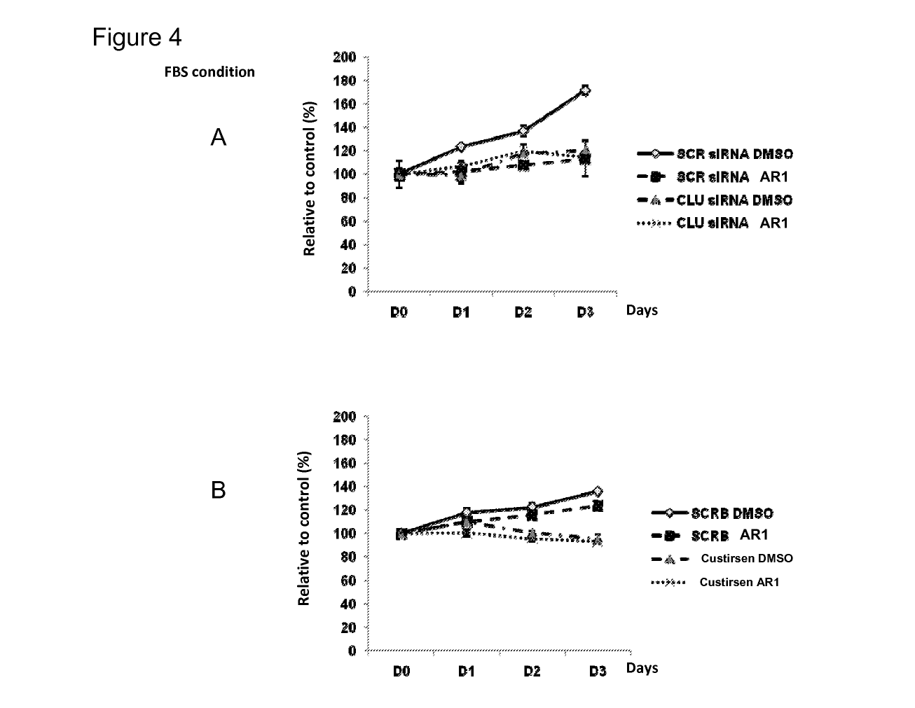

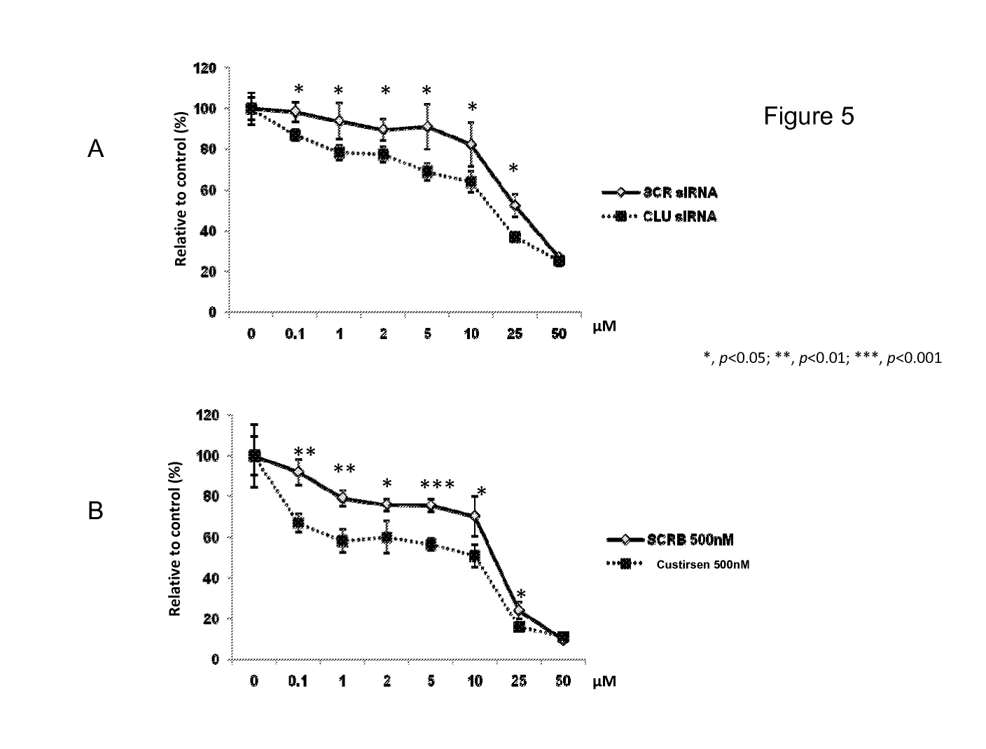

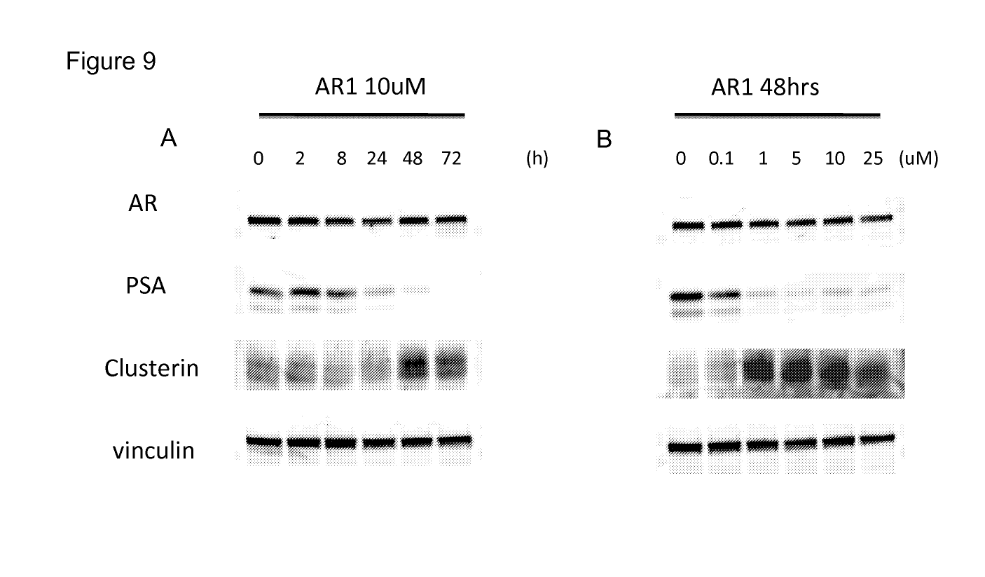

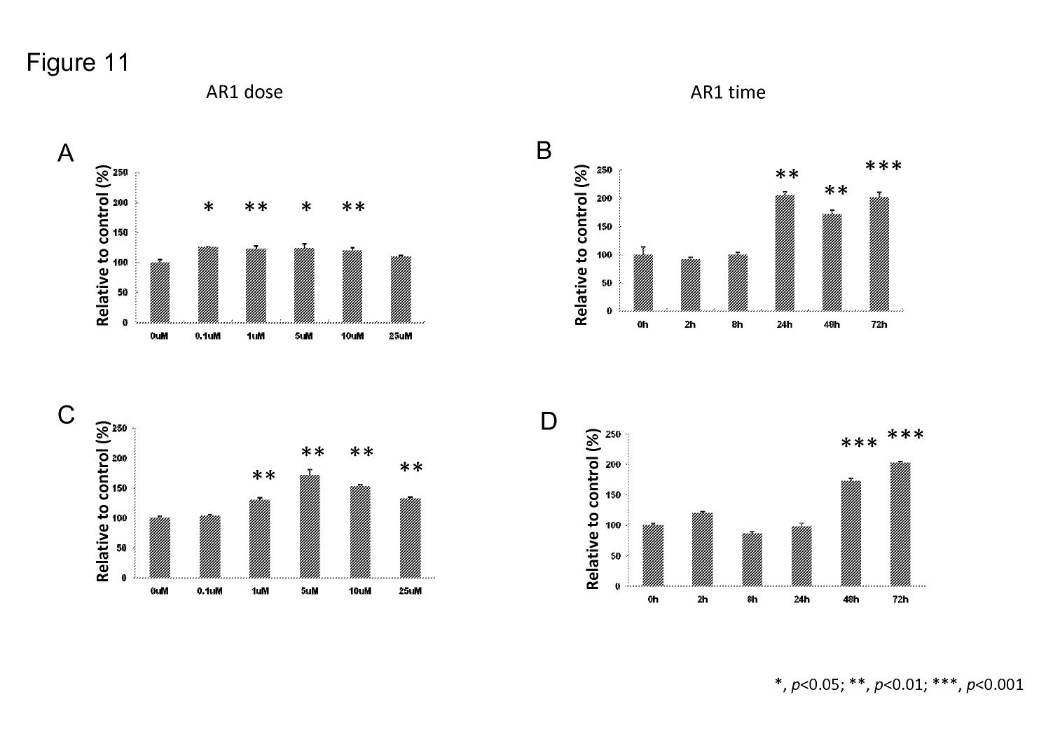

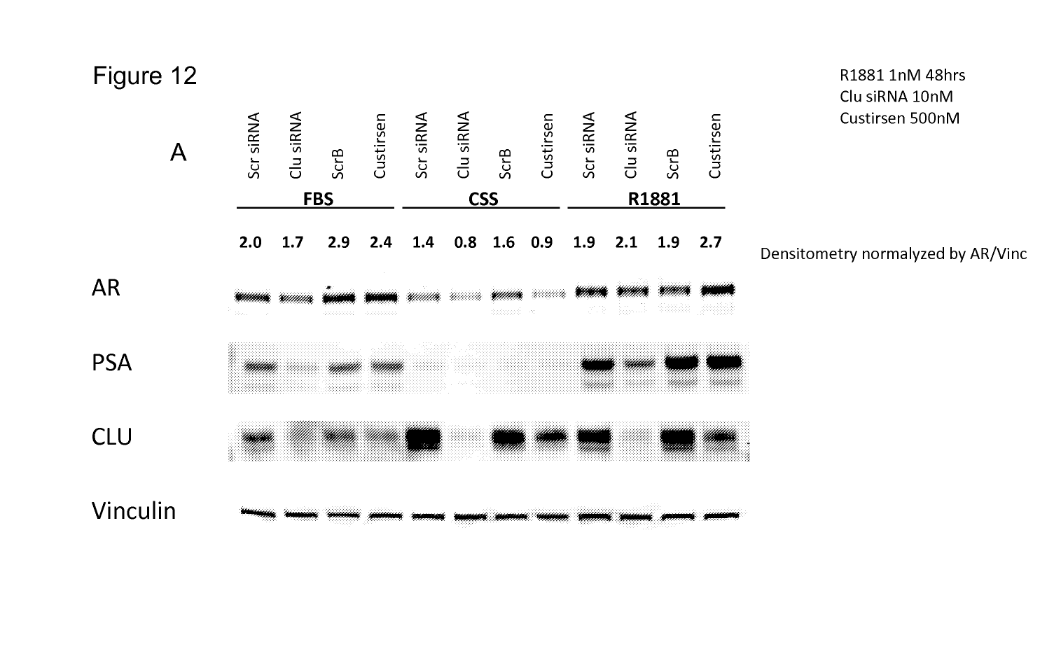

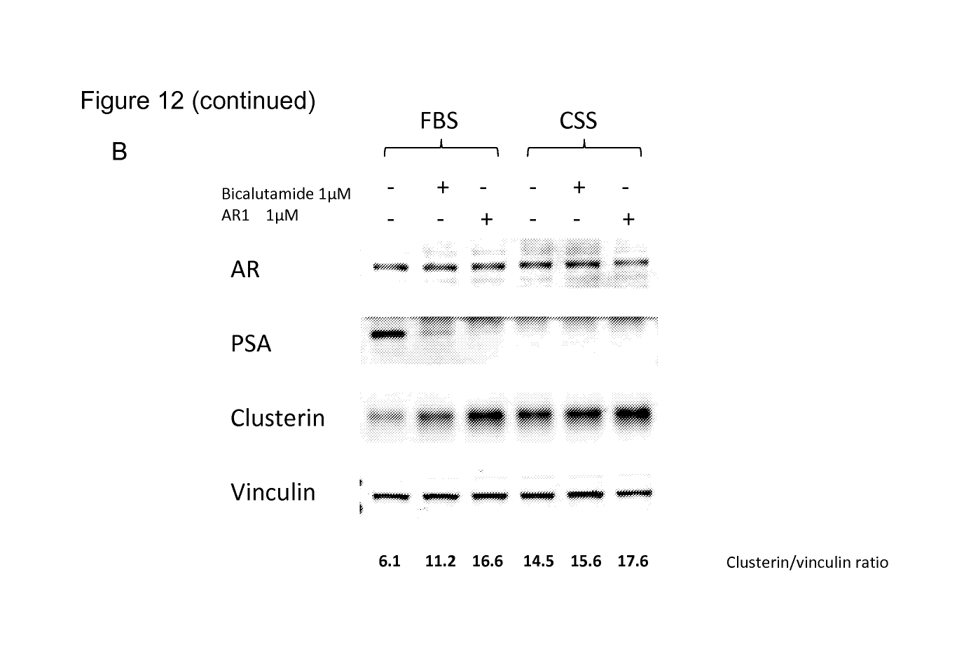

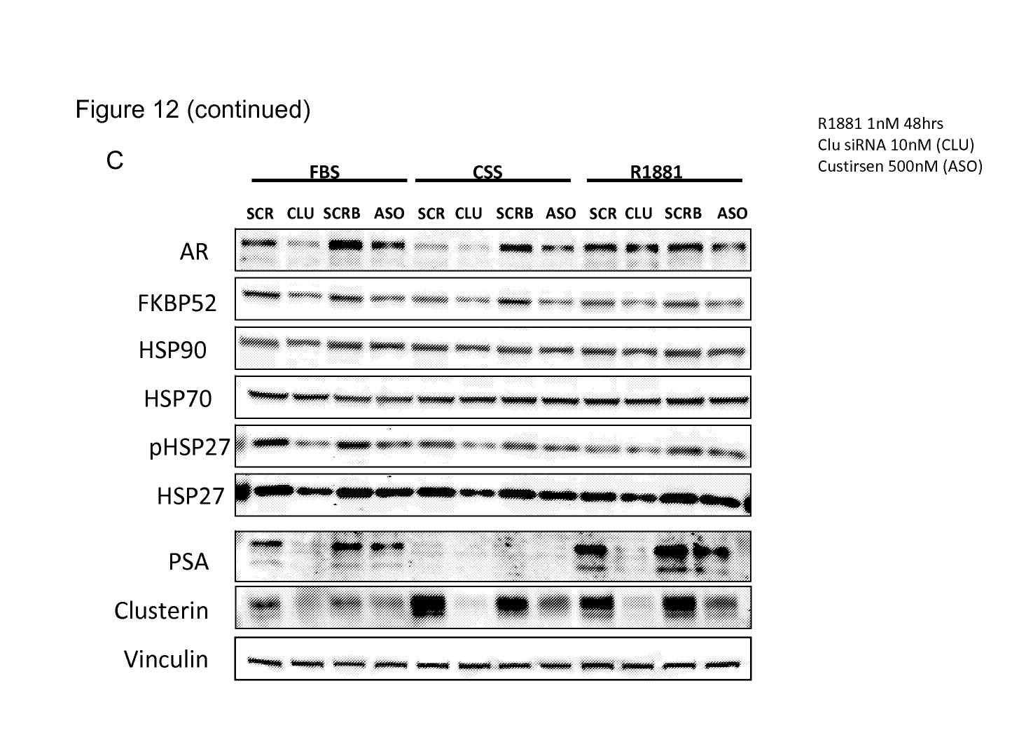

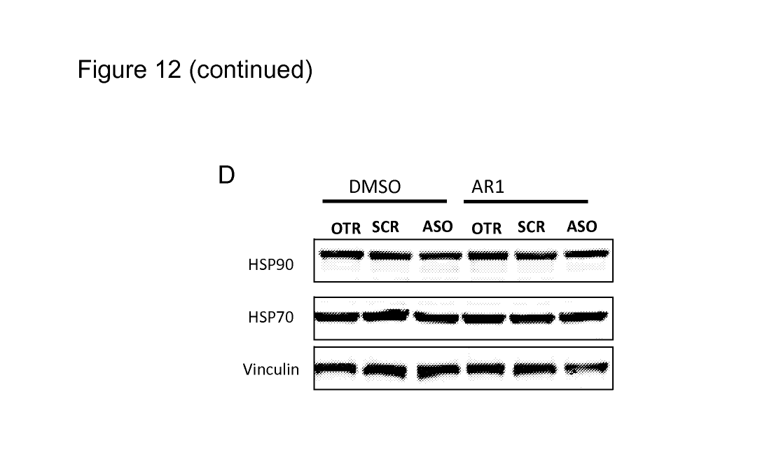

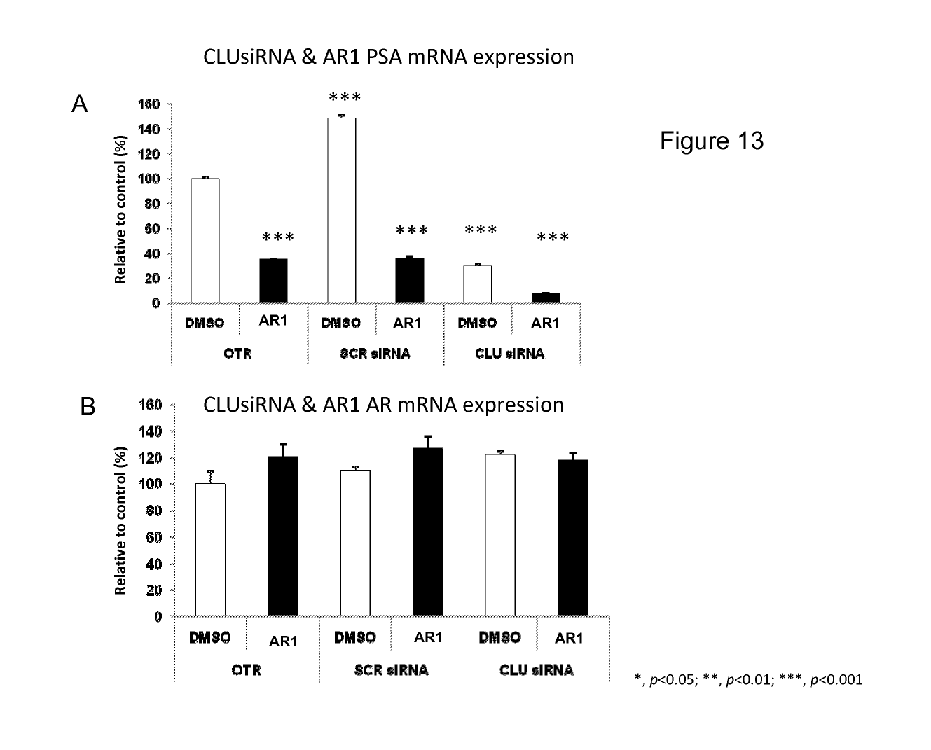

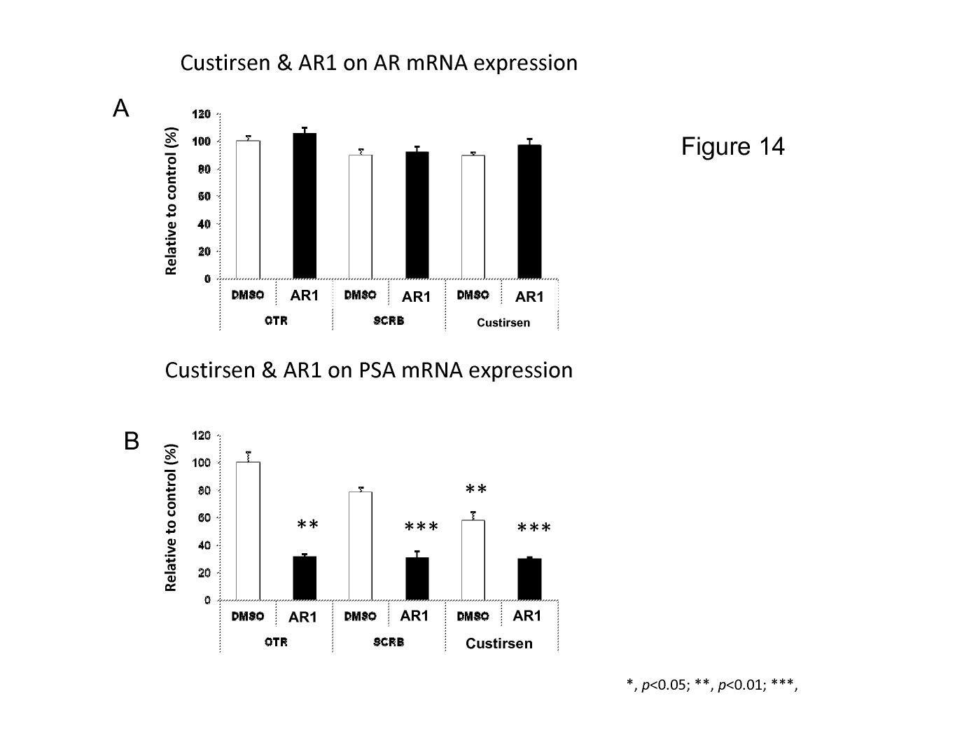

Effects of individual vs combination AR1 and custirsen regimens on AR-positive LNCaP cell proliferation (FIGS. 1-4, and 6-7) and survival (FIG. 5) as well as protein (FIGS. 9, 11, and 13-15), and gene expression (FIGS. 13 and 14) were analyzed using a crystal violet assay, flow cytometry, western blotting and RT-PCR, respectively. AR transcriptional activity was measured by a PSA-luciferase reporter assay, while AR degradation was assessed by a cycloheximide chase assay. The LNCaP cell line used for experiments was AR positive.

The effects of combination treatment on castration-resistant LNCaP tumor growth were assessed in castrated male athymic nude mice. Male athymic nude mice were inoculated with LNCaP cells in Matrigel in two sites of mouse flank lesion. The mice were castrated once tumors reached 150 mm³ or the PSA level increased above 50 ng/mL. Once tumors progressed to castration resistance (PSA levels increased to the same level as pre-castration), 10 mice were randomly assigned to each of AR1+scrambled antisense oligonucleotide (SCRB) or AR1+custirsen treatment groups. Custirsen (10 mg/kg/each dose) or SCRB (10 mg/kg/each dose) was injected i.p. once daily for the first week and then three times per week. AR1 (10 mg/kg/each dose) was administered orally once daily (morning) 7 days per week for 8 to 12 weeks. Tumor volume was measured once per week. Serum PSA was determined weekly. PSA doubling time (PSAdt) and velocity were calculated by the log-slope method (PSAₜ−PSAᵢₙᵢₜᵢₐₗ×e^{mt}). All animal procedures were performed according to the guidelines of the Canadian Council on Animal Care and appropriate institutional certification.

### Results

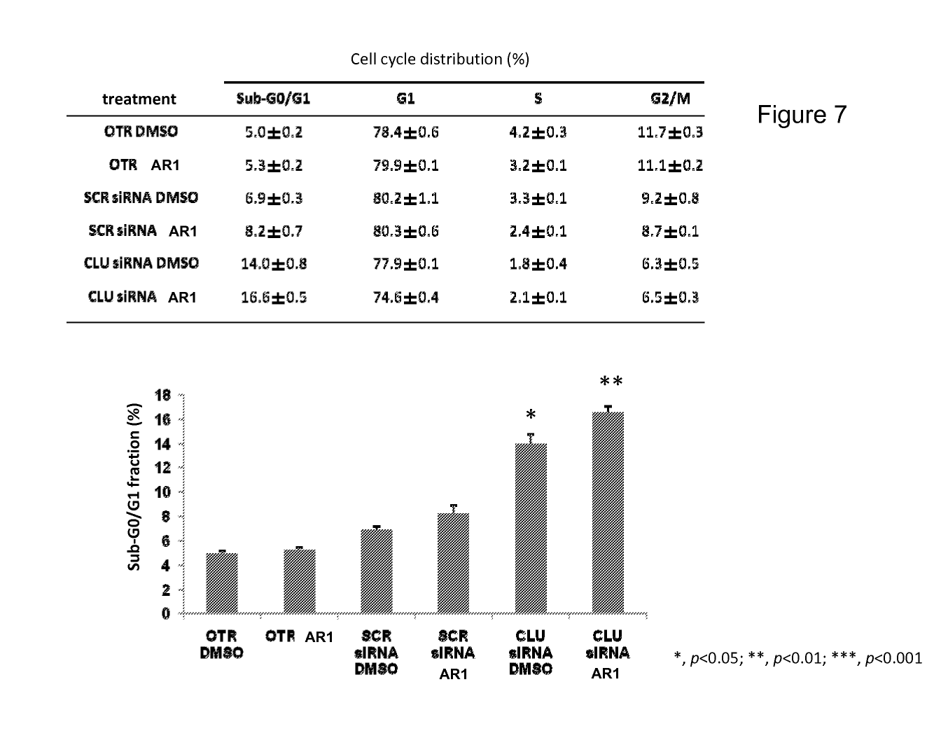

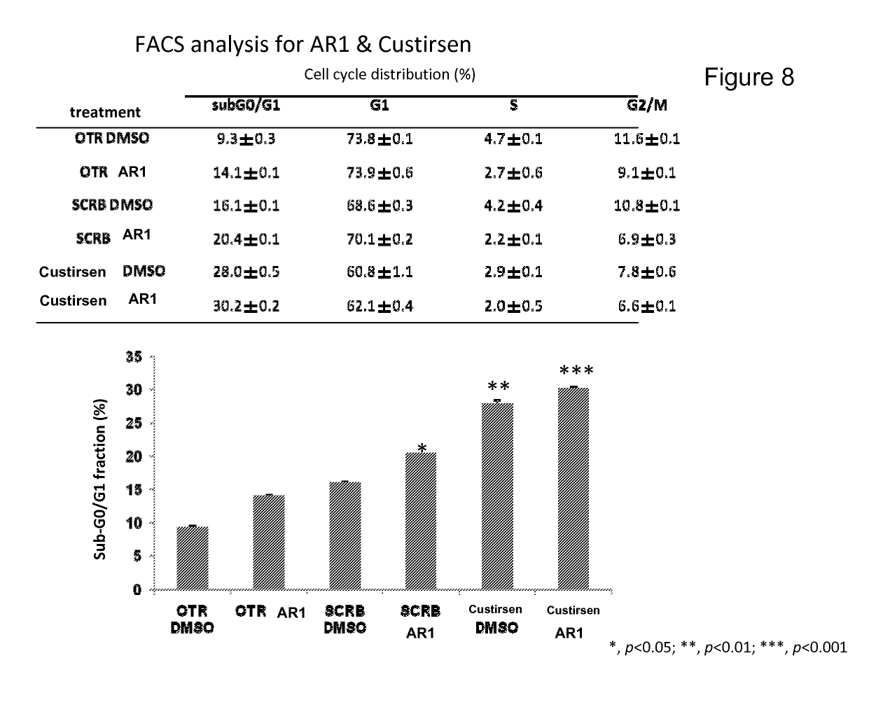

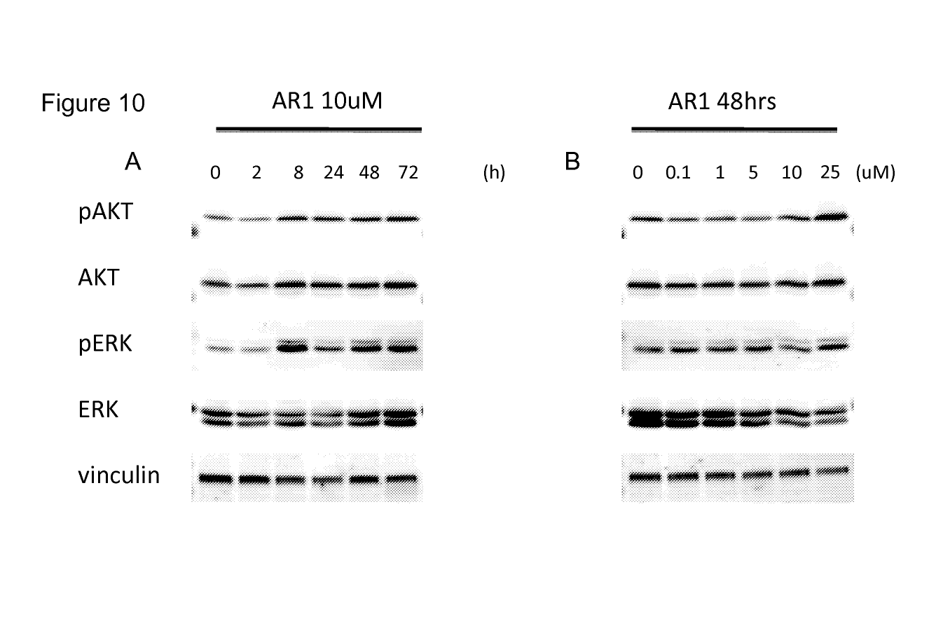

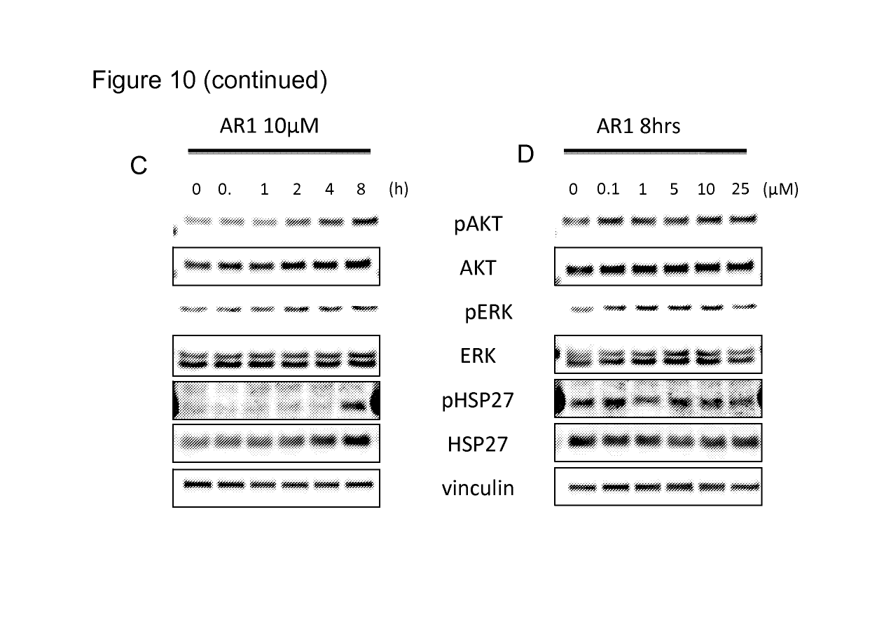

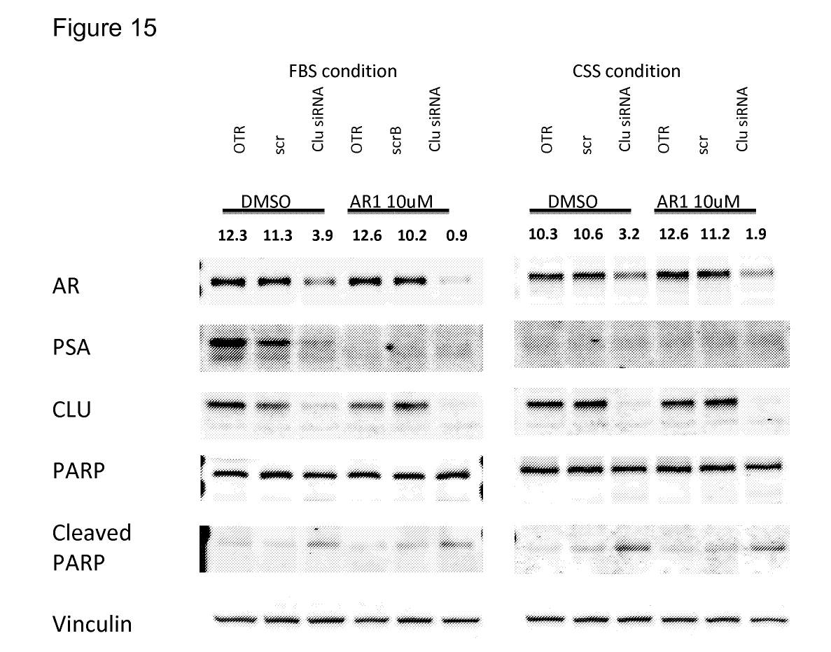

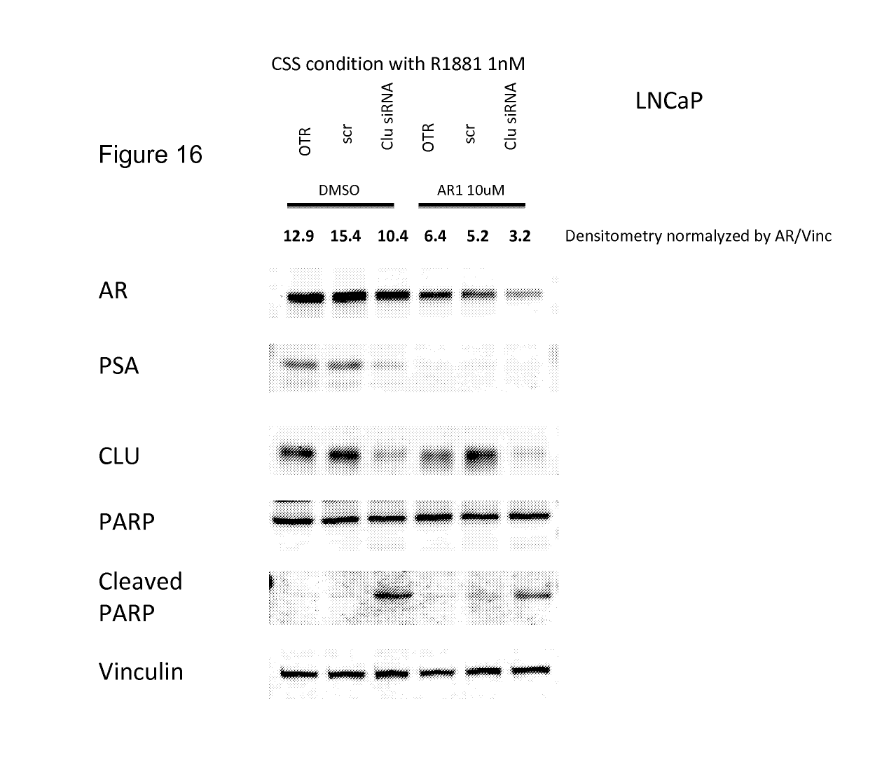

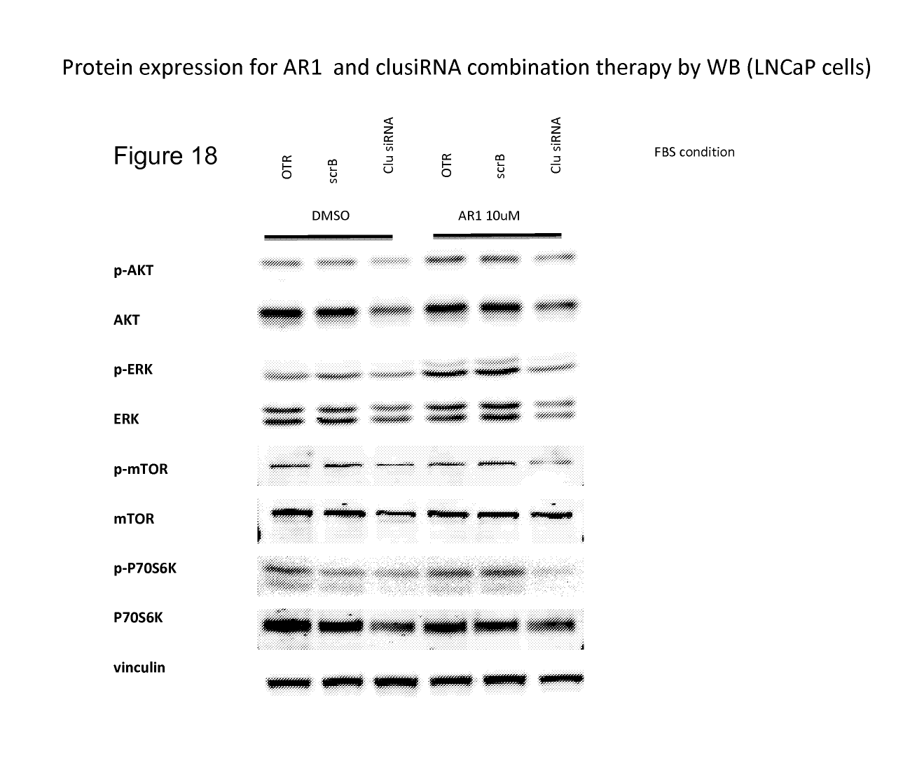

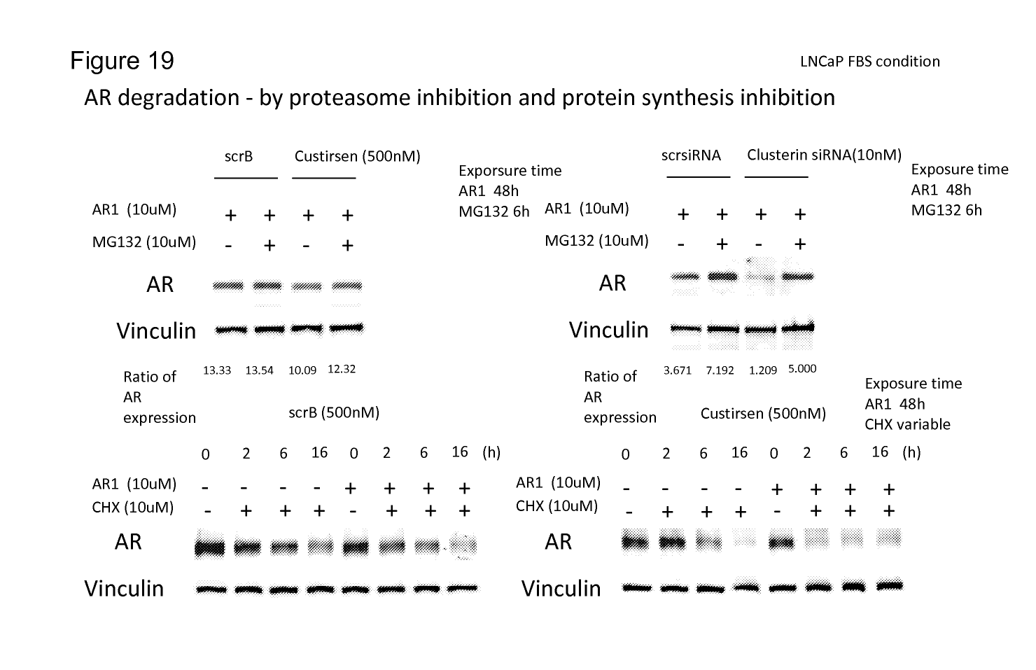

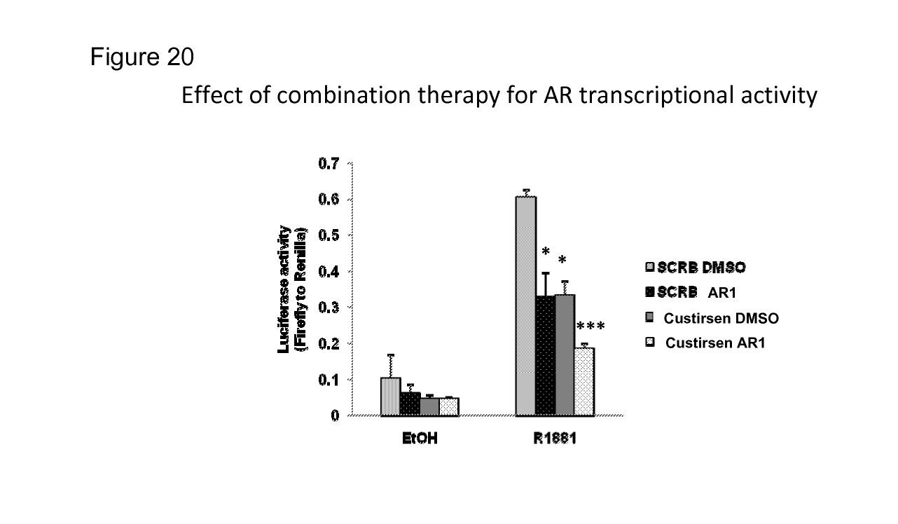

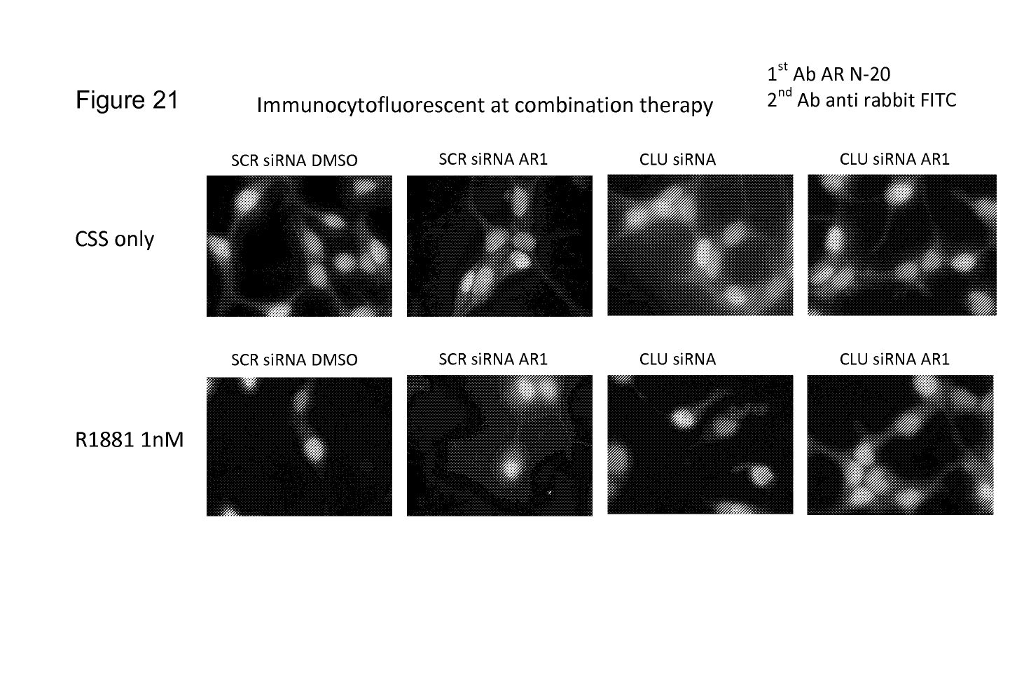

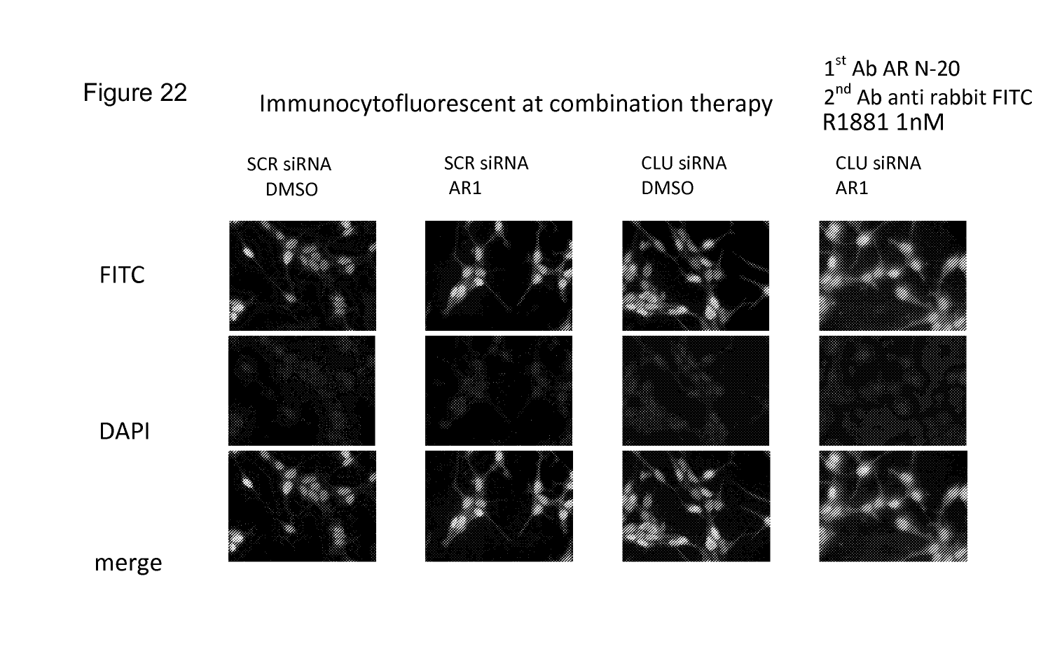

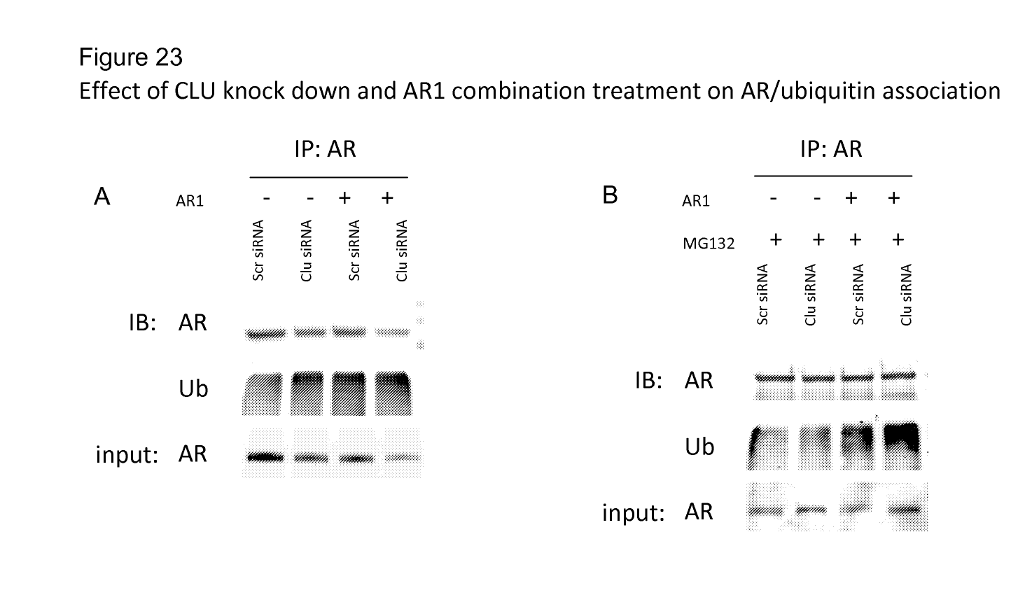

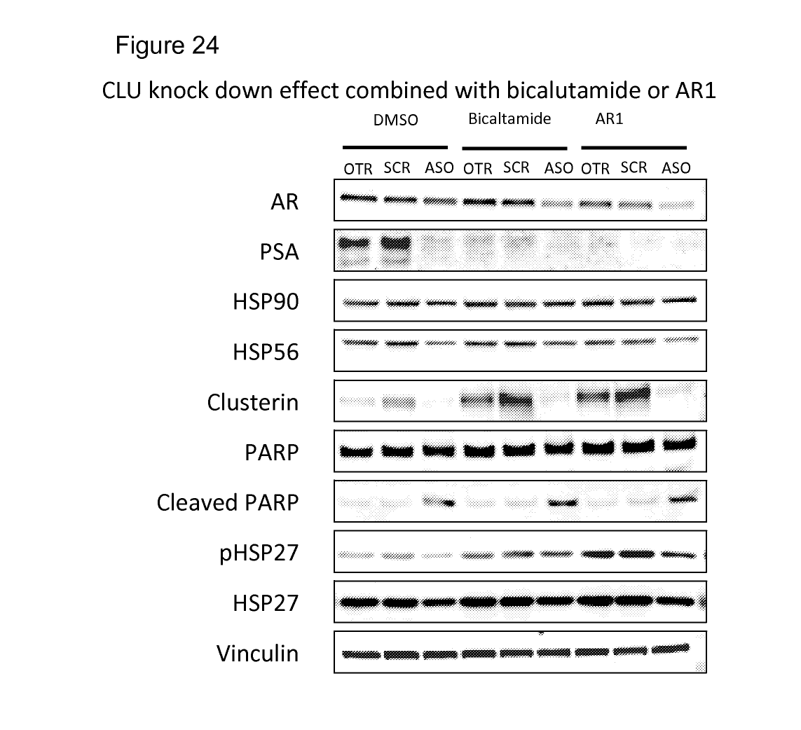

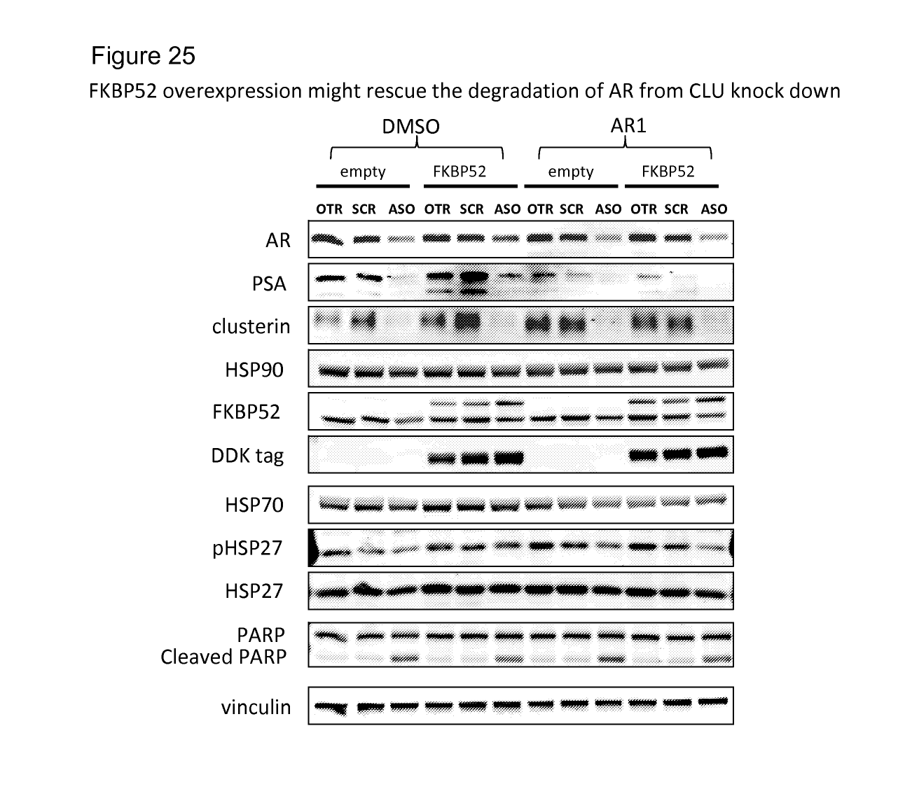

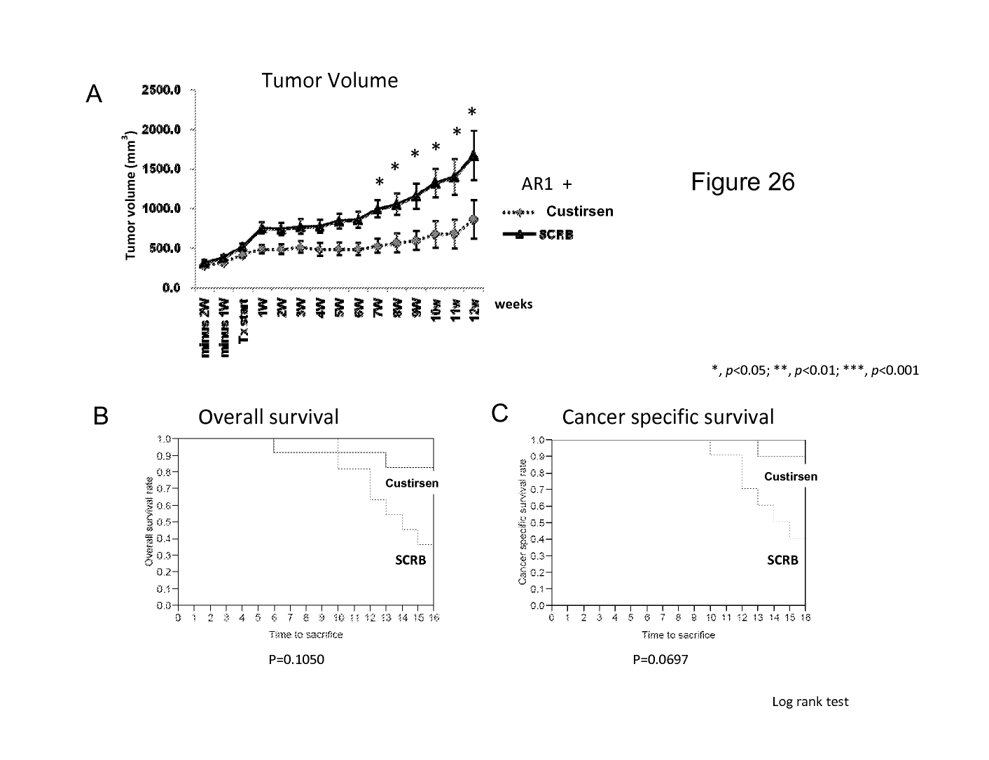

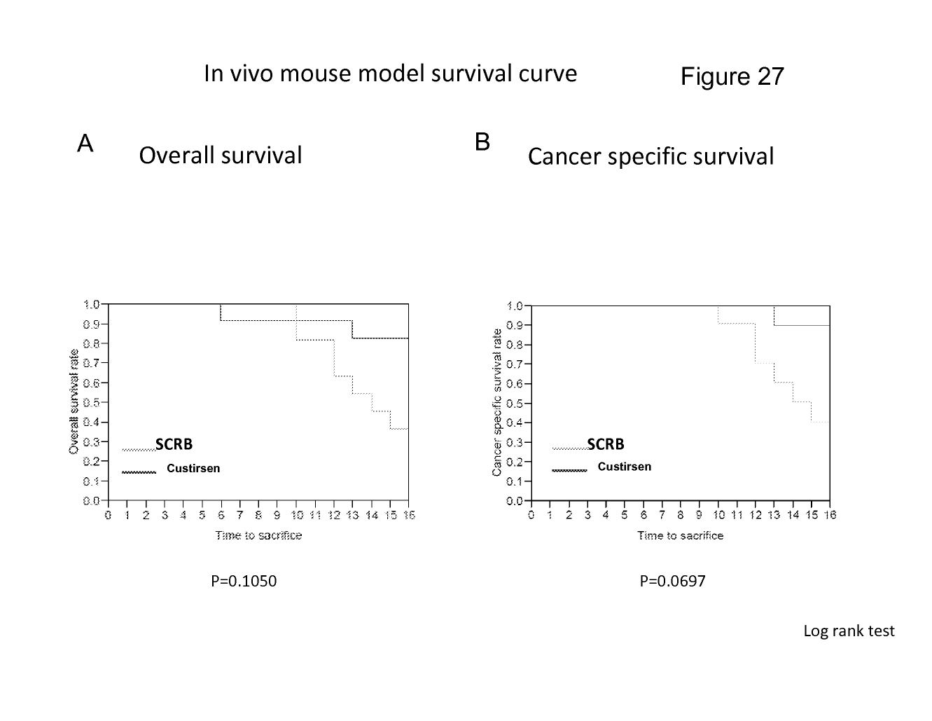

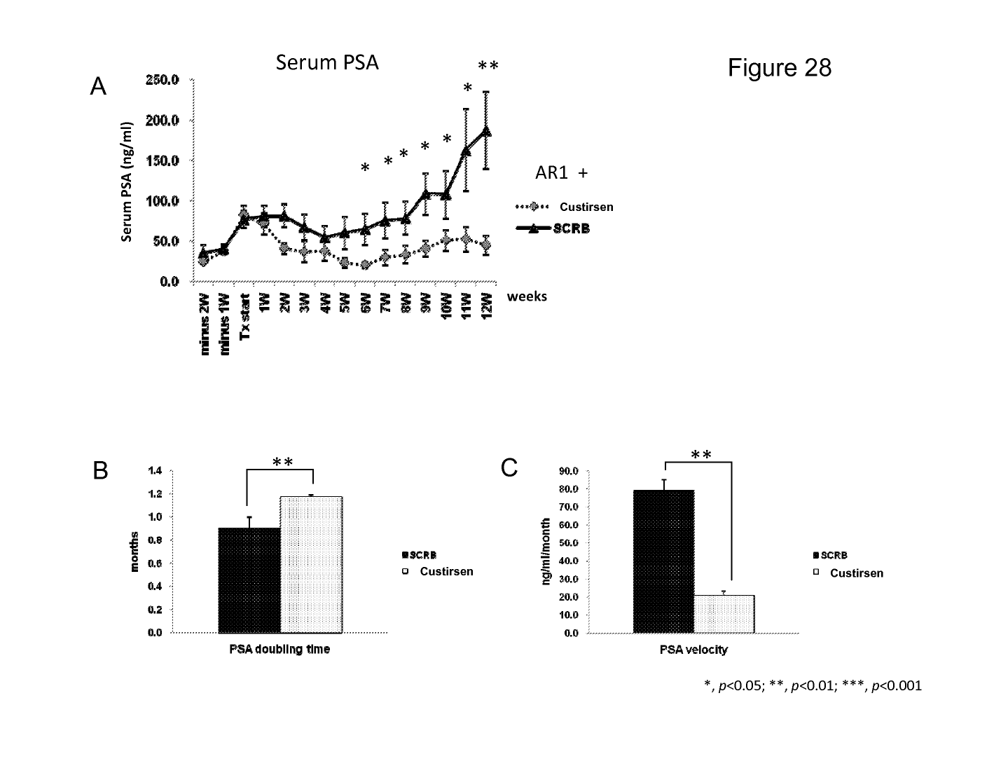

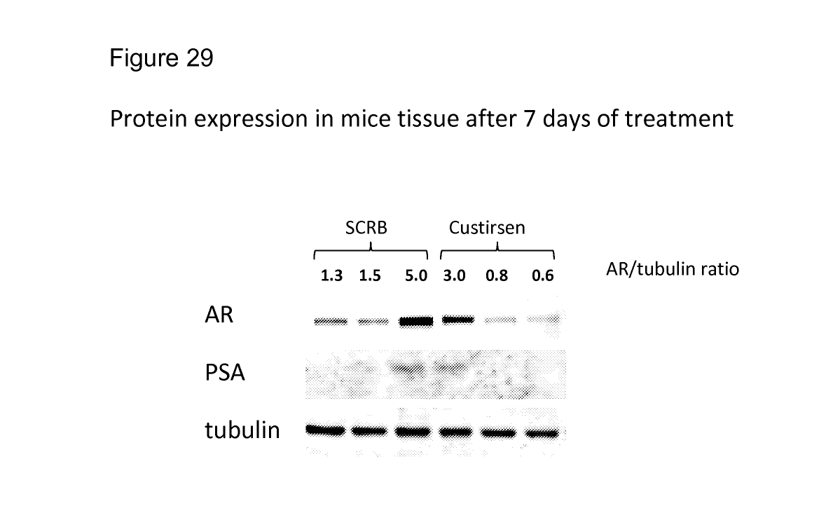

The combination of custirsen and AR1 more potently suppressed LNCaP cell growth rates in a dose and time dependent manner compared to custirsen or AR1 monotherapy (FIGS. 1-8). Surprisingly, PARP cleavage (FIG. 15), sub G0/G1 apoptotic fraction (FIGS. 7 and 8) and repressed AKT phosphorylation (FIGS. 10 and 18) was most increased with combined therapy. Additionally, custirsen accelerated AR degradation (FIGS. 15-19 and 22) and repressed AR transcriptional activity (FIGS. 20-22) in combination with AR1. In vivo, combined custirsen and AR1 significantly delayed castration-resistant LNCaP tumor progression and PSA progression (FIGS. 26-29) compared to scrambled oligonucleotide control and AR1 (p<0.05 and p<0.05 at 12 weeks, respectively).

### Conclusions

Custirsen combined with AR1 down-regulated AR levels and activity and suppressed castration-resistant LNCaP cell growth in vitro and in vivo, providing pre-clinical proof-of-principle as a promising approach for AR-targeting therapy in CRPC.

### Example 2

### Materials and Methods

### Prostate Cancer Cell Lines and Reagents

LNCaP cells were kindly provided by Dr. Leland W. K. Chung (1992, MDACC, Houston Tx) and tested and authenticated by whole-genome and whole-transcriptome sequencing on Illumina Genome Analyzer IIx platform in July 2009. LNCaP cells were maintained RPMI 1640 (Invitrogen Life Technologies, Inc.) supplemented with 5% fetal bovine serum and 2 mmol/L L-glutamine. Cells were cultured in a humidified 5% CO₂/air atmosphere at 37° C. Cycloheximide and MG-132 were purchased from Calbiochem, R1881 (Perkin-Elmer), AR1 (MDV-3100; Haoyuan Chemexpress Co., Limited). Antibodies: anti-GRP78, anti-CREB2 (ATF4), CLU C-18, AR N-20, AR 441, PSA C-19, Ubiquitin, pERK, β-tubulin and vinculin from Santa Cruz Biotechnology; anti-phospho-eIF2α from Invitrogen Life Technologies; anti-ATF6 from Imgenex Corp; Atg3, LC3, pAkt/Akt, pmTOR/mTOR, pp70S6K/p70S6K, poly(ADP ribose)polymerase (PARP)form from Cell Signaling Technology; and anti-Vinculin and anti-β-Actin from Sigma-Aldrich.

CLU siRNA and Antisense Treatment

siRNAs were purchased from Dharmacon Research, Inc., using the siRNA sequence corresponding to the human CLU initiation site in exon 2 and a scramble control as previously described (Lamoureux et al., 2011). Second-generation antisense (custirsen) and scrambled (ScrB) oligonucleotides with a 2′-O-(2-methoxy)ethyl modification were supplied by OncoGenex Pharmaceuticals. custirsen sequence (5′-CAGCAGCAGAGTCTTCATCAT-3′; SEQ ID NO: 3) corresponds to the initiation site in exon II of human CLU. The ScrB control sequence was 5′-CAGCGCTGACAACAGTTTCAT-3′ (SEQ ID NO: 44). Prostate cells were treated with siRNA or oligonucleotides, using protocols described previously (Lamoureux et al., 2011).

### Western Blotting Analysis and Immunoprecipitation

Total proteins were extracted using RIPA buffer (50 mM Tris, pH 7.2, 1% NP-40, 0.1% deoxycholate, 0.1% SDS, 100 mM NaCl, Roche complete protease inhibitor cocktail) and submitted to western blot as we described previously (Zoubeidi et al., 2007). For immunoprecipitation, total proteins (500 μg) were pre-cleared with protein-G sepharose (Invitrogen Life Technologies) for 1 h at 4° C. and immunoprecipitated with 2 μg anti-AR, or immunoglobulin G (IgG) as a control overnight at 4° C. The immune complexes were recovered with protein-G sepharose for 2 h and then washed with radioimmunoprecipitation assay buffer (RIPA) at least thrice, centrifuged, and submitted to SDS-PAGE, followed by Western blotting.

### Quantitative Reverse Transcription-PCR

Total RNA was extracted from cultured cells after 48 hours of treatment using TRIzol reagent (Invitrogen Life Technologies, Inc.). Two μg of total RNA was reversed transcribed using the Transcriptor First Strand cDNA Synthesis Kit (Roche Applied Science). Real time monitoring of PCR amplification of complementary DNA (cDNA) was performed using DNA primers (supplemental table) on ABI PRISM 7900 HT Sequence Detection System (applied Biosystems) with SYBR PCR Master Mix (Applied Biosystems). Target gene expression 5′-TACCAGCTCACCAAGCTCCT-3′ (forward; SEQ ID NO: 45) or 5′-GCTTCACTGGGTGTGGAAAT-3′ (reverse; SEQ ID NO: 46) targeting human AR, 5′-CACAGCCTGTTTCATCCTGA-3′ (forward; SEQ ID NO: 47) or 5′-AGGTCCATGACCTTCACAGC-3′ (reverse; SEQ ID NO: 48) targeting human PSA, were normalized to b-actin levels using 5′-AAATCTGGCACCACACCTTC-3′ (forward; SEQ ID NO: 49) or 5′-AGCACTGTGTTGGCGTACAG-3′ (reverse; SEQ ID NO: 50) as an internal standard, and the comparative cycle threshold (Ct) method was used to calculated relative quantification of target mRNAs. Each assay was performed in triplicate.

### Immunofluorescence

LNCaP cells were grown on coverslips and transfected with CLU siRNA or control. 48 hours post transfection cells were treated with 10 uM of AR1±1 nM R1881 for 6 hours. After treatment, cells were fixed in ice-cold methanol completed with 3% acetone for 10 min at −20° C. Cells were the washed thrice with PBS and incubated with 0.2% Triton/PBS for 10 min, followed by washing and 30 min blocking in 3% nonfat milk before the addition of antibody overnight to detect AR (1:250). Antigens were visualized using anti-mouse antibody coupled with FITC (1:500; 30 min). Photomicrographs were taken at 20× magnification using Zeiss Axioplan II fluorescence microscope, followed by analysis with imaging software (Northern Eclipse, Empix Imaging, Inc.).

### AR Transcription Activity

LNCaP cells were seeded at a density of 5×10⁴ in 12-well plates and transfected the following day with custirsen or SCRB. The next day cells were transfected with custirsen or SCRB together with PSA-luciferase (PSA-Luc) reporter (−6,100 to +12) and Renilla-luciferase plasmid using Lipofectin reagent (1.5 μL per well; Invitrogen), as described previously (Sowery et al., 2008). After 24 h, the medium was replaced with RPMI (Invitrogen) containing 5% charcoal-stripped serum (CSS), supplemented with 1 nmol/L R1881 or ethanol vehicle and 10 μmol/L of AR1 or DMSO for 48 h. Cells were harvested, and luciferase activity measured, as before (Sowery et al., 2008). Reporter assays were normalized to Renilla and luciferase activity expressed by Firefly to Renilla ratio in arbitrary light units. All experiments were carried out in triplicate wells and repeated five times using different preparations of plasmids.

### Cell Proliferation and Cell Cycle Assays

Cultured cells were transfected with CLU or SCR siRNA, custirsen or SCRB, and then treated with AR1 or DMSO control 24 h after transfection. After a time course exposure, cell growth was measured by crystal violet assay as previously described (cleave et al., 2005). Detection and quantitation of apoptotic cell cycle population were analyzed by flow-cytometry (Beckman Coulter Epics Elite; Beckman, Inc.) based on 2N and 4N DNA content as previously described (Lamoureux et al., 2011). For CSS condition, LNCaP cells were plated in RPMI with 5% FBS switched to CSS at the next day, and treatment was started same as FBS condition. Each assay was done in triplicate three times.

### Protein Stability and Degradation

To assess the effect of combination treatment on AR protein stability, LNCaP cells treated with custirsen or SCRB were changed 48 h later to RPMI+5% serum containing 10 μmol/L of cycloheximide and 10 μmol/L of AR1 incubated at 37° C. for 2 to 6 or 16 h and western blot was done using AR and vinculin antibodies. Degradation was tested in LNCaP cells by a 6-hour incubation with RPMI+5% FBS media containing 10 μmol/L of MG132 and 10 μmol/L AR1 24 hours after siRNA or ASO transfection. Western blot was done using AR and vinculin antibodies.

### Determination of Increased Efficacy of Combination Therapy

Crystal violet assay was applied to analyze cell growth inhibition for each single drug or their combination. LNCaP cells were treated with 10 nmol/L CLU siRNA or SCR siRNA combined with escalation dose of AR1 and 500 nmol/L custirsen or SCRB as well. Next, cells were treated for two consecutive days with dose escalating custirsen or oligofectamime only, and one day later treated with indicated concentration of AR1 or DMSO for 48 h. The data was inputted in CalcuSyn® software, and the dose effect curve drawn for each treatment to calculate the combination index (CI) at several effective doses (CI=1; additive effect, CI<1; combination effect, CI>1; antagonistic effect).

### Animal Treatment

Male athymic mice (Harlan Sprague-Dawley, Inc.) were injected s.c. with 1×10⁶ LNCaP cells. When tumors grow in 150 mm³ and serum PSA was >50 ng/ml, mice are castrated. Once tumors progressed to castrate resistance, mice were randomly assigned to AR1 plus either 10 mg/kg custirsen or SCRB i.p. once daily for 7 days and then three times per week thereafter. Each experimental group consisted of 13 mice. Simultaneously, mice were treated with AR1 once daily p.o., 10 mg/kg/each dose for 7 days per week. Tumor volume and serum PSA was measured as previously described (Sowery et al., 2008). All animal procedures were performed according to the guidelines of the Canadian Council on Animal Care. Each three mice xenografts were sacrificed at 7 days after start treatment and the rest were harvested the end of the study and snap-frozen in liquid nitrogen. Protein extraction was done by soliciting tumors in RIPA buffer with protease inhibitor and total cell lysate was used to assess AR and clusterin expression within the xenografts and referenced for β-tubulin as described in the section on Western blotting.

### Statistical Analysis

All results are expressed as the average ±SE. Two-tailed t-tests, one-way ANOVA or Wilcoxon matched-pairs tests were used for statistical analysis. Combination effects were calculated by CalcuSyn software. The differences between single treatment and combination treatment was analysis by Freidman test and done with JMP version 4; *P<0.05, **P<0.01, and ***P<0.001 were considered significant.

### Example 3

### CLU is Highly Expressed in AR1 Resistant Cells and Xenografts

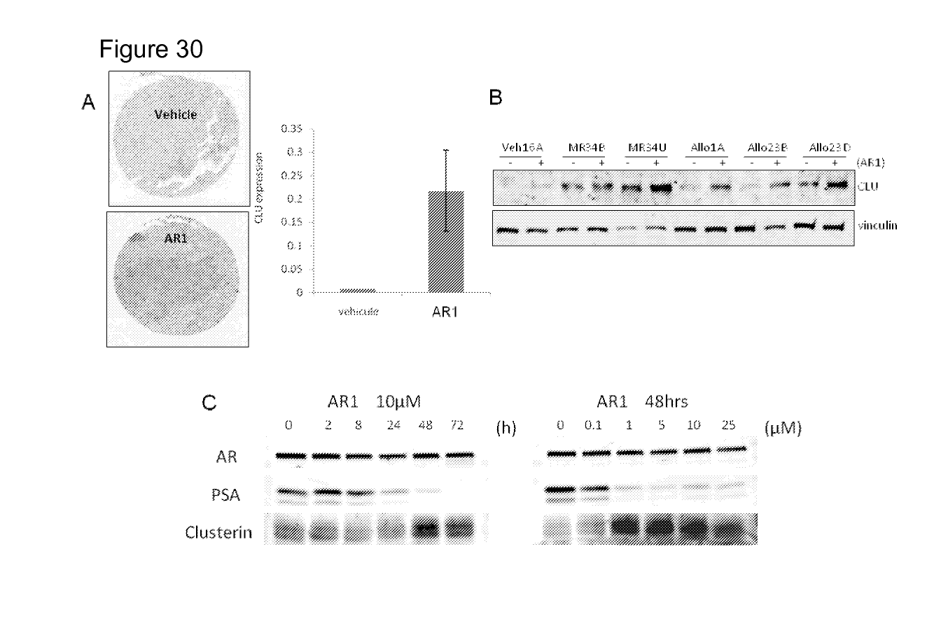

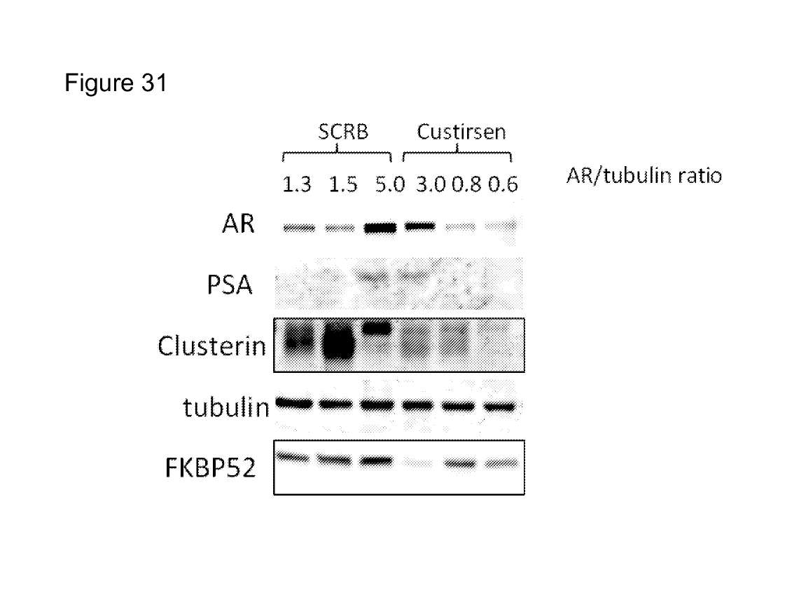

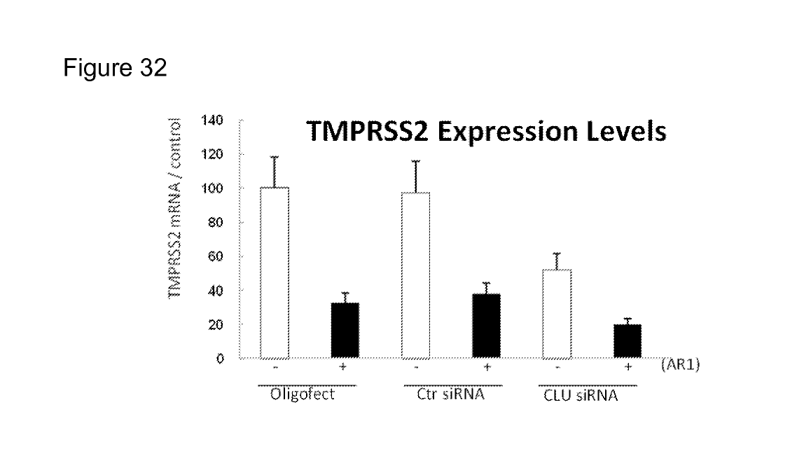

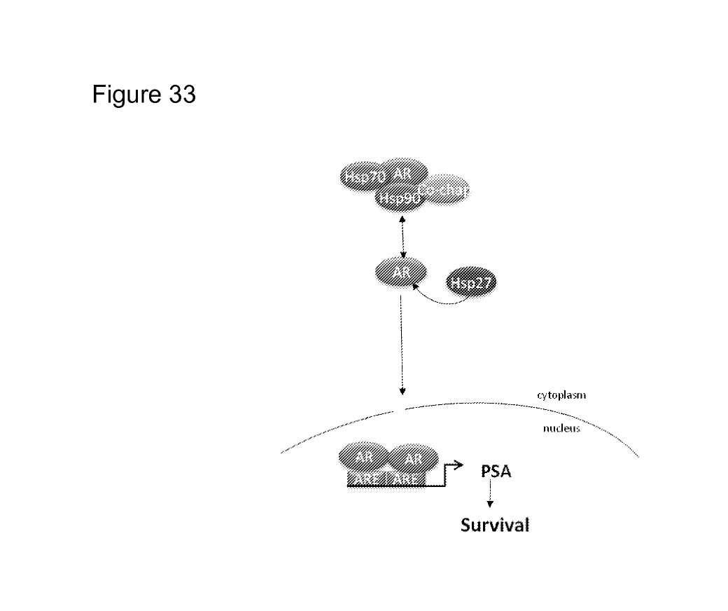

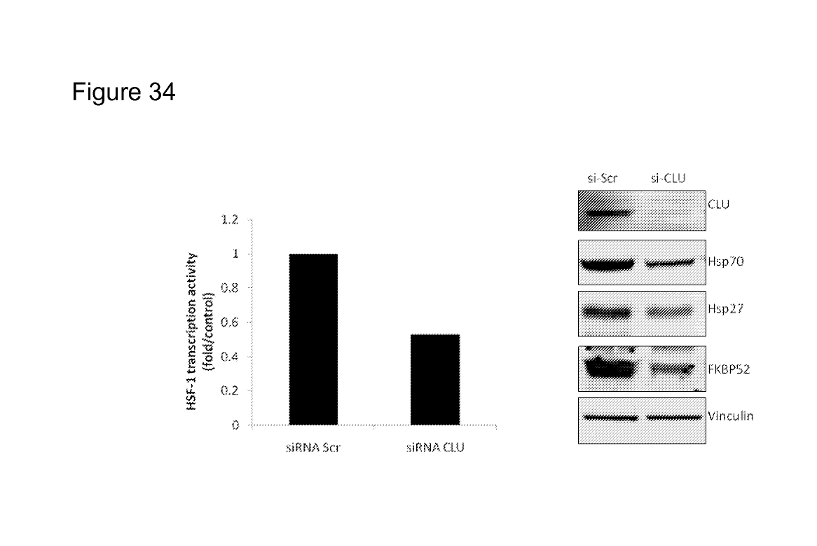

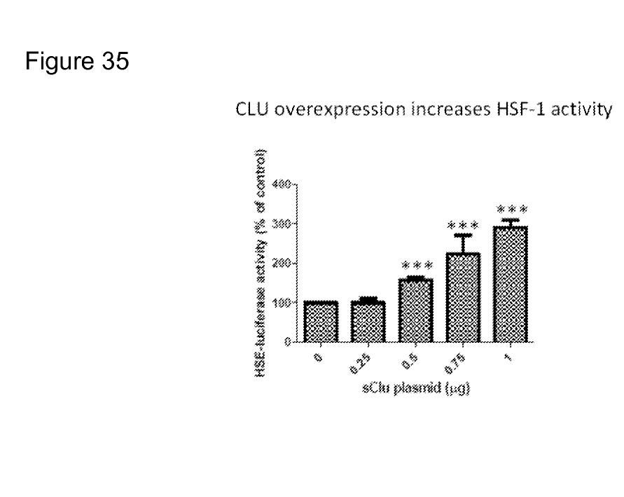

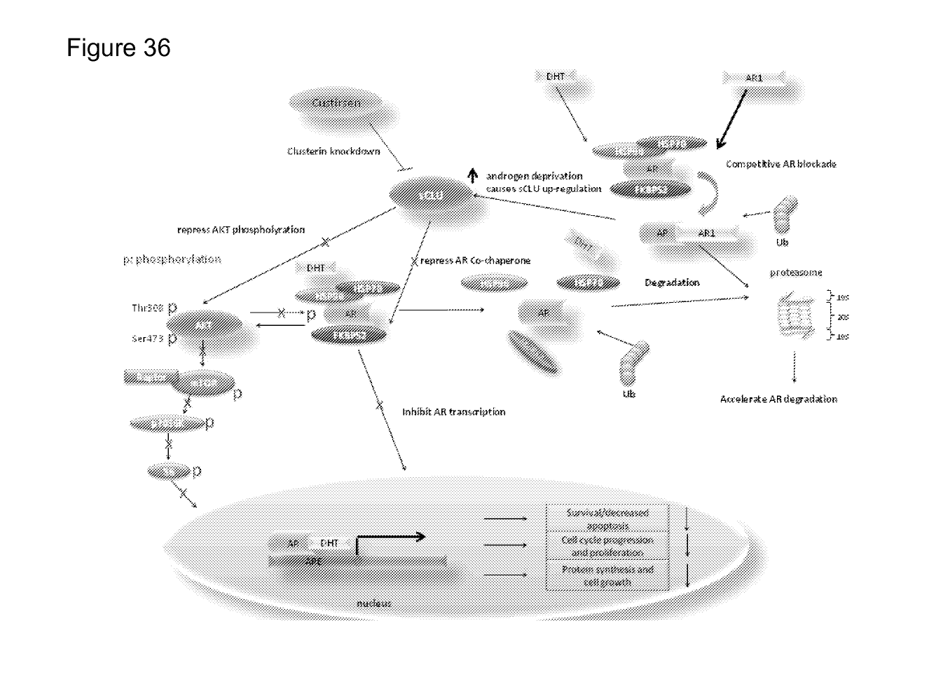

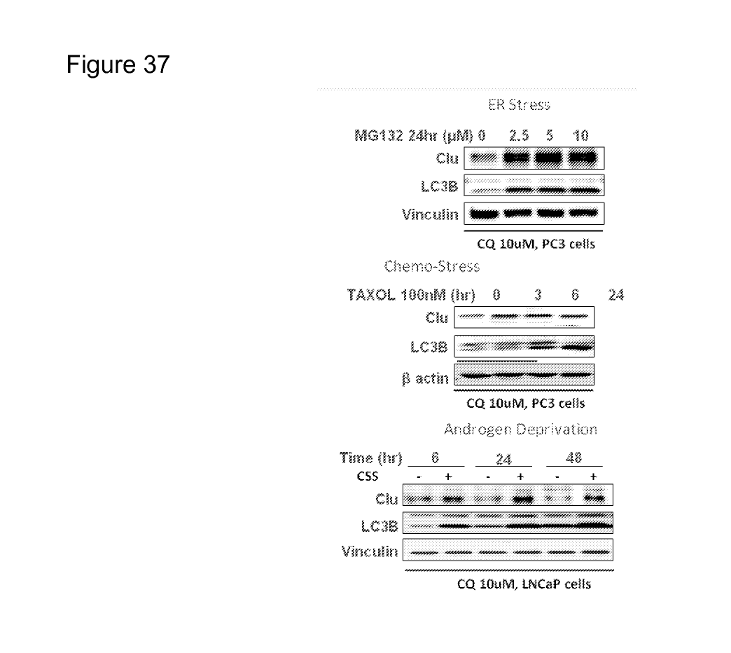

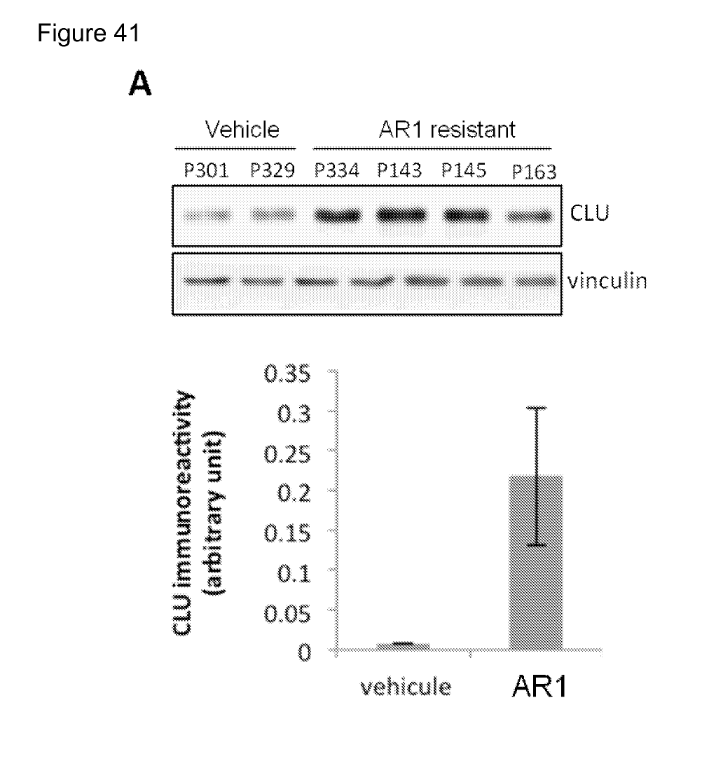

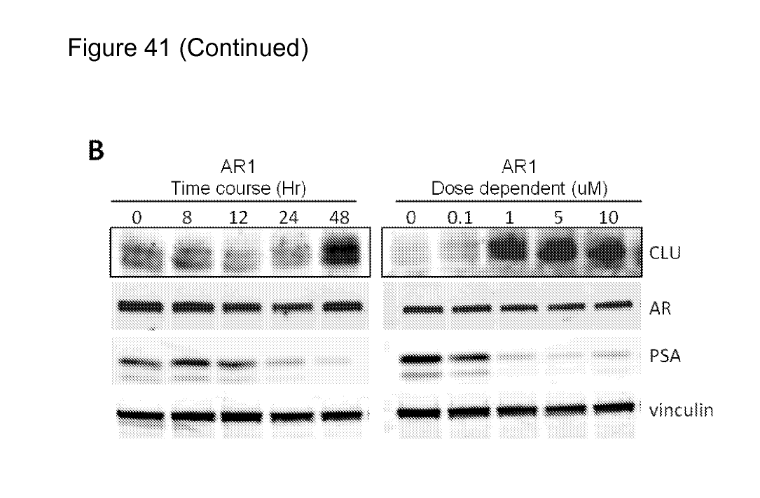

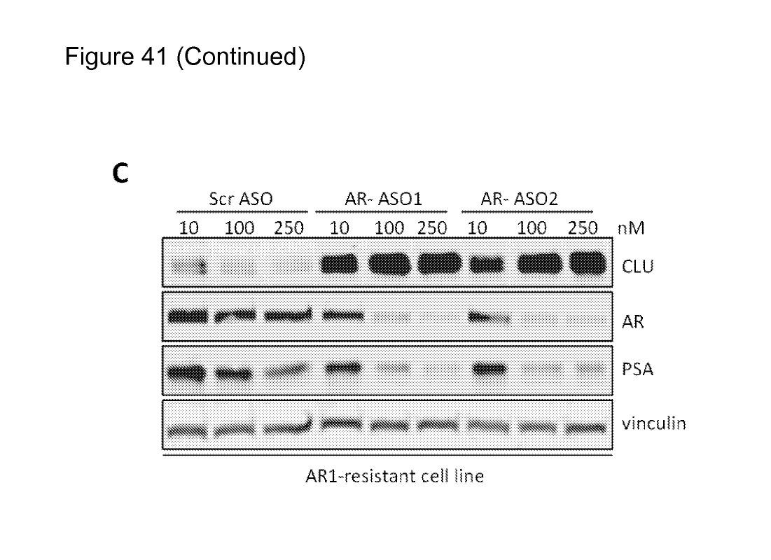

AR1 is a novel anti-androgen which binds the AR LBD and inhibits the growth of castration-resistant xenografts (Tran et al., 2009). Data from phase II and III trials show that AR1 is active in both pre- and post-chemotherapy-treated patients and decreases levels of PSA and circulating tumor cells (Scher et al., 2010) (Sher, GU-ASCO, 2012). Unfortunately, like first line hormone therapies, CRPC-LNCaP xenografts evolved mechanisms of resistance after the addition of AR1 to castration. CLU was found to be up-regulated in AR1 resistant tumors compared to vehicle treated tumors by western blot (FIG. 41A, left panel) and immunohistochemistry (FIG. 41A right panel, FIG. 30A), suggesting that AR1 treatment induces stress activated molecular chaperone CLU in CRPC tumors similar to that seen with castration in castrate sensitive tumors. To facilitate study of mechanisms of AR1 recurrence, different cell lines were created from xenograft tumors maintained under AR1 treatment that were resistant to AR1, and found that these AR1 resistant cells also expressed higher levels of CLU compared to CRPC tumors (FIG. 30B). These data indicate that increased CLU is associated with development of the AR1 recurrence phenotype.

### Example 4

### AR Pathway Inhibition Induces and CLU

Both AR1 antisense approaches were used to confirm whether CLU is induced by AR pathway inhibition. Compared to bicalutamide and androgen deprivation using CSS, AR1 induces CLU in both a time- and dose-dependent manner, in parallel with reduced AR activation indicated by decreased PSA expression. To further evaluate whether AR1 induction of CLU is AR dependent, 2 different antisense sequences targeting the first exon in AR potently down-regulated AR in a dose-dependent and sequence-specific manner in LNCaP cells, in parallel with induction of CLU (FIG. 41C). Together these data suggest that AR pathway inhibition by androgen deprivation, AR LBD antagonism, or antisense knockdown induces CLU, possibly as part of an adaptive stress response.

Clusterin expression is up-regulated by AR1 treatment. Clusterin expression is up-regulated in a time and dose dependent manner after AR1 treatment. LNCaP Cells are treated for different durations and with different concentrations of AR1 in RPMI1640 media with 5% FBS. Cells are harvested and performed for western blot analysis. Clusterin protein expression is up regulated in a time and dose dependent manner. Androgen depleted treatment enhances clusterin expression, especially in AR1. LNCaP Cells are treated with 10 μmol/L of Bicalutamide or AR1 for 48 hrs in RPMI1640 media with 5% FBS or 5% CSS (charcoal striped serum; testosterone depleted media). Clusterin expression is strongly induced by anti-androgen treatment or CSS condition. Additionally, clusterin protein expression strongly increases in AR1 treatment compared to bicalutamide treatment in western blot analysis. The combination of AR1 and custirsen is more effective at reducing prostate cancer cell proliferation than the combination of bicalutamide and custirsen.

### Example 5

### AR1 Induces ER Stress

Whether AR1 induces ER stress with increase of CLU was evaluated since molecular chaperones like CLU are important in regulating misfolded protein and endoplasmic reticular (ER) stress responses (Nizard et al., 2007), and many anti-cancer agents are known to induce ER stress. ER stress activates a complex intracellular signaling pathway, called the unfolded protein response (UPR), which is tailored to re-establish protein homeostasis (proteostasis) by inhibiting protein translation and promoting ER-associated protein degradation via the ubiquitin-proteasome system (UPS). AR1 is found to induce CLU expression concomitant with up-regulation of ER stress markers such as GRP78, ATF4, IRE1, CHOP and cleaved-ATF6, consistent with ER stress and UPR activation.

### Example 6

### AR1-Induced CLU is Mediated by p90Rsk-YB-1 Signalling Pathway

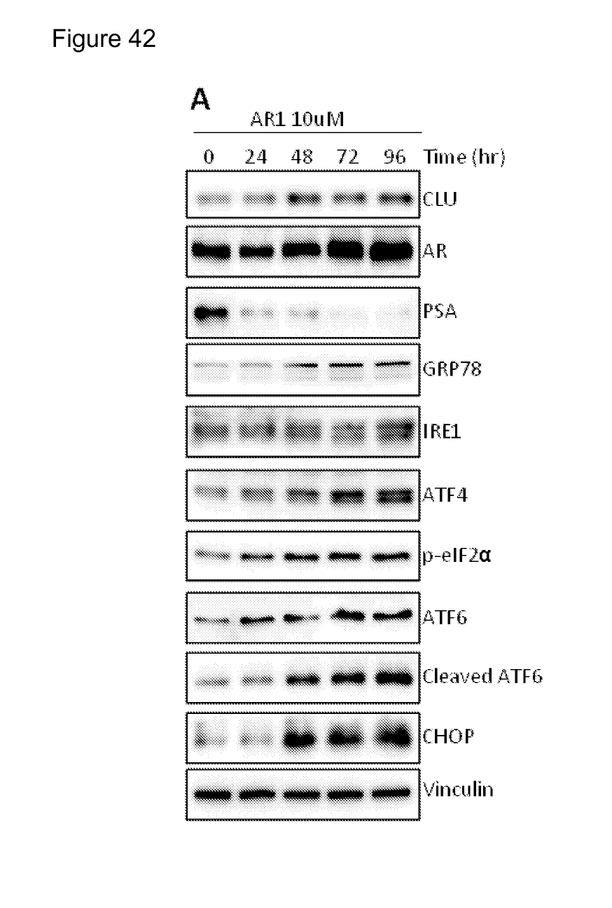

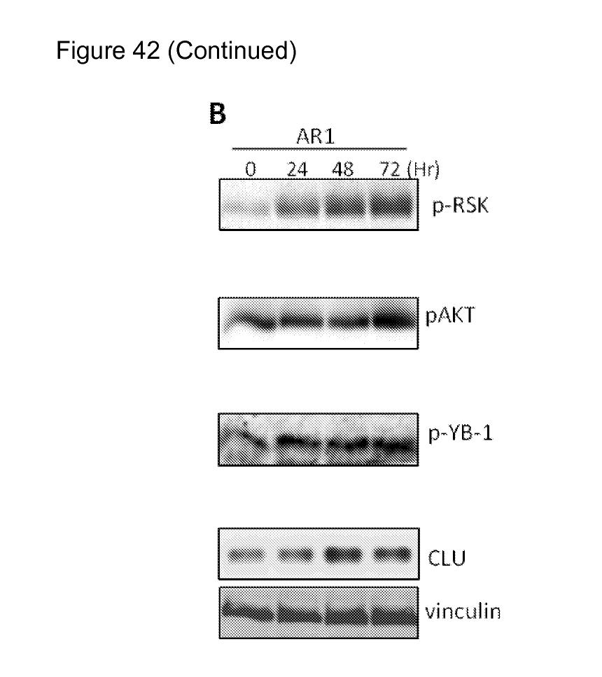

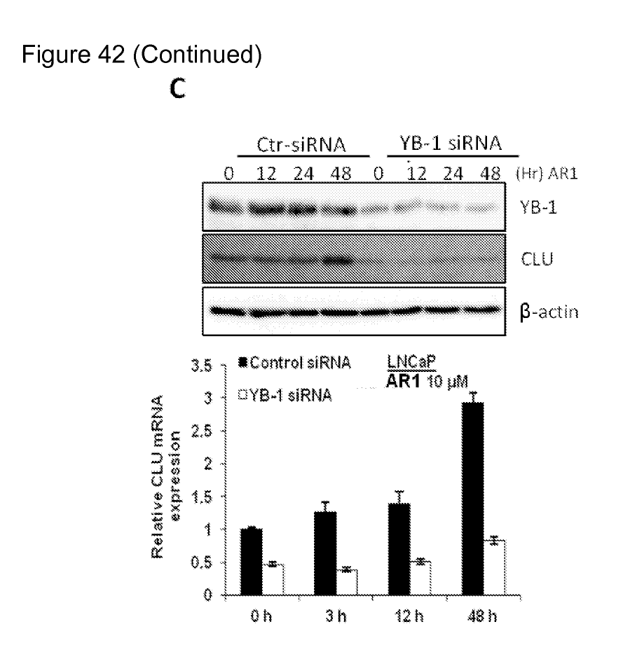

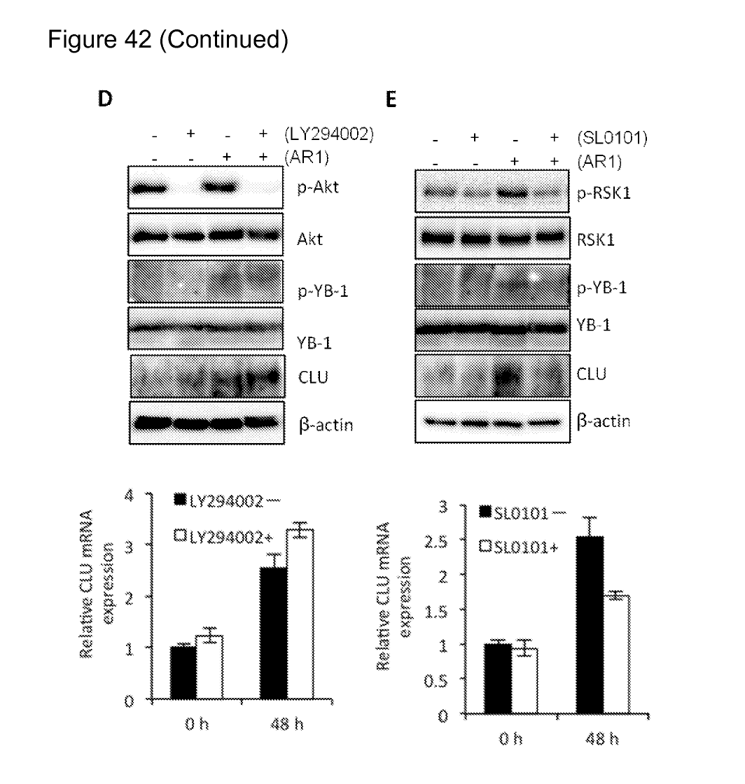

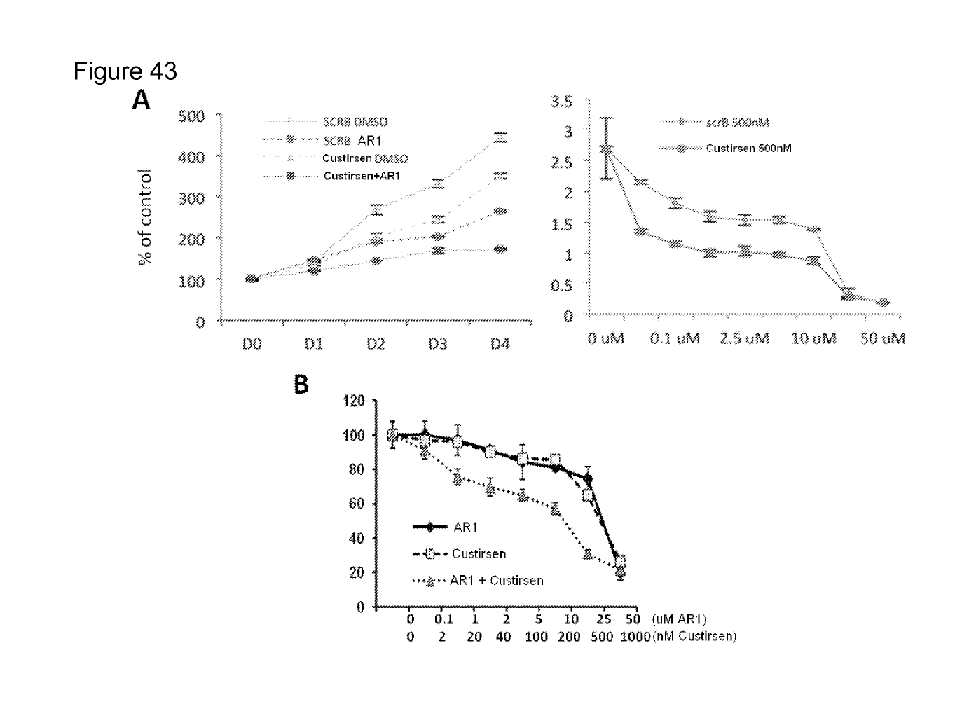

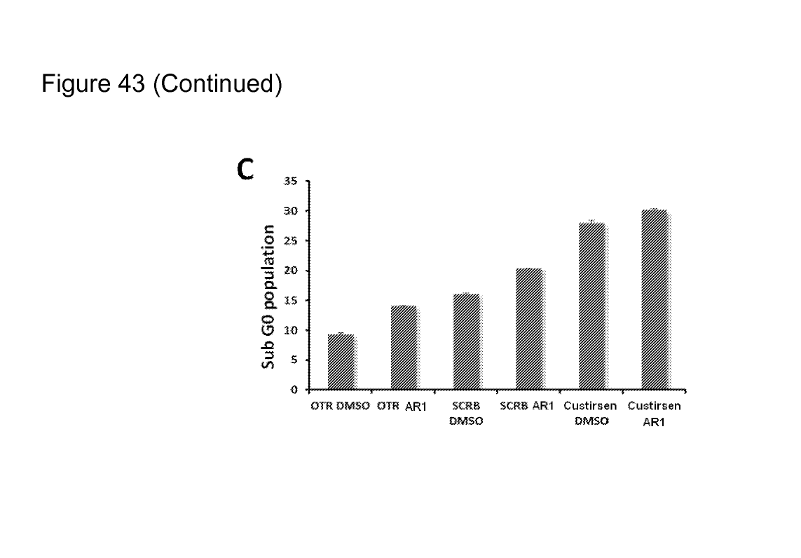

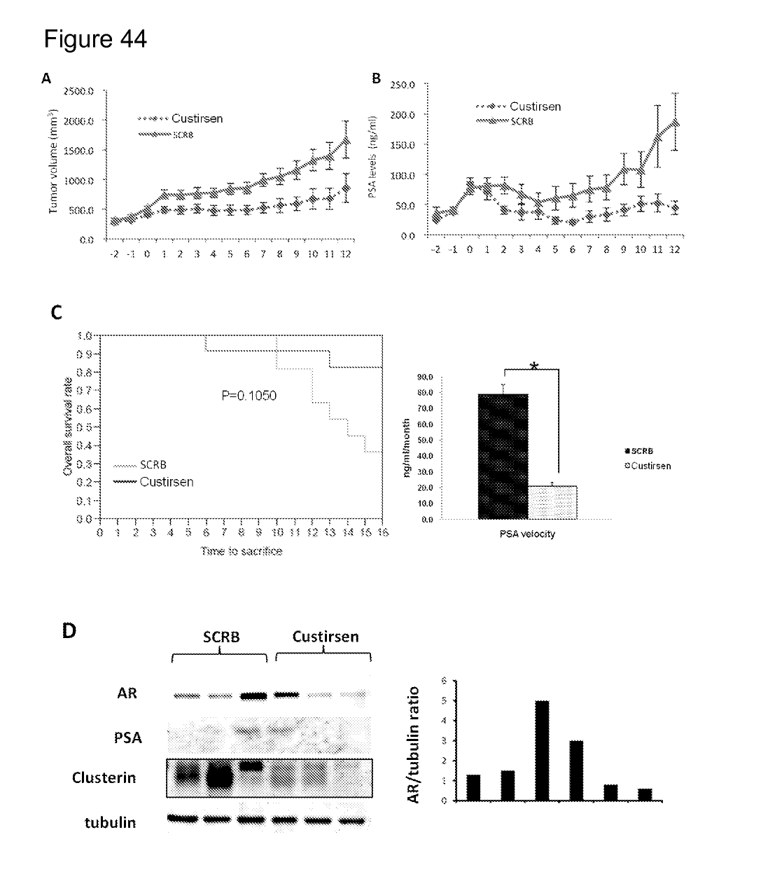

YB-1 binds to CLU promoter leading to increased CLU expression after ER stress (Shiota et al., 2011). Since AR1 can activate Akt and Erk signalling (Carver et al., 2011), it was postulated that AR1 mediated activation of Akt (Evdokimova et al., 2006) and Erk (Stratford et al., 2008) pathways leads to phospho-activation and nuclear translocation of YB-1 (Evdilomova et al., 2006), with up-regulation of CLU and inhibition of stress-induced apoptosis. FIG. 42B confirms that AR1 treatment increases Akt and p90Rsk phosphorylation, which was accompanied with increased phospho-YB-1 levels (FIG. 42B). YB-1 knockdown using siRNA in combination with AR1 treatment abrogates AR1-induced CLU both at the protein and mRNA levels (FIG. 42C) suggesting that AR1-induced CLU is mediated by YB-1.

Since YB-1 can be phosphorylated by both Akt and p90Rsk (Evdilomova et al., 2006) (Stratford et al., 2008), LY294002 was used to inhibit Akt and SL0101 was used to inhibit p90Rsk to further define the predominant pathway mediating AR1 induced up-regulation of CLU. Inhibition of Akt did not affect AR1 induced up-regulation of CLU (FIG. 42D); in contrast, inhibition of p90Rsk using SL101 abrogates AR1-induced CLU (FIG. 42E). Without wishing to be bound by any scientific theory, collectively these data indicate that the p90Rsk-YB-1 pathway is required for AR1 induced CLU expression.

### Example 7

### The Combination of CLU Inhibition and AR1 Increases Inhibition of LNCaP Cell Growth Compared to CLU Inhibition or AR1 Monotherapy

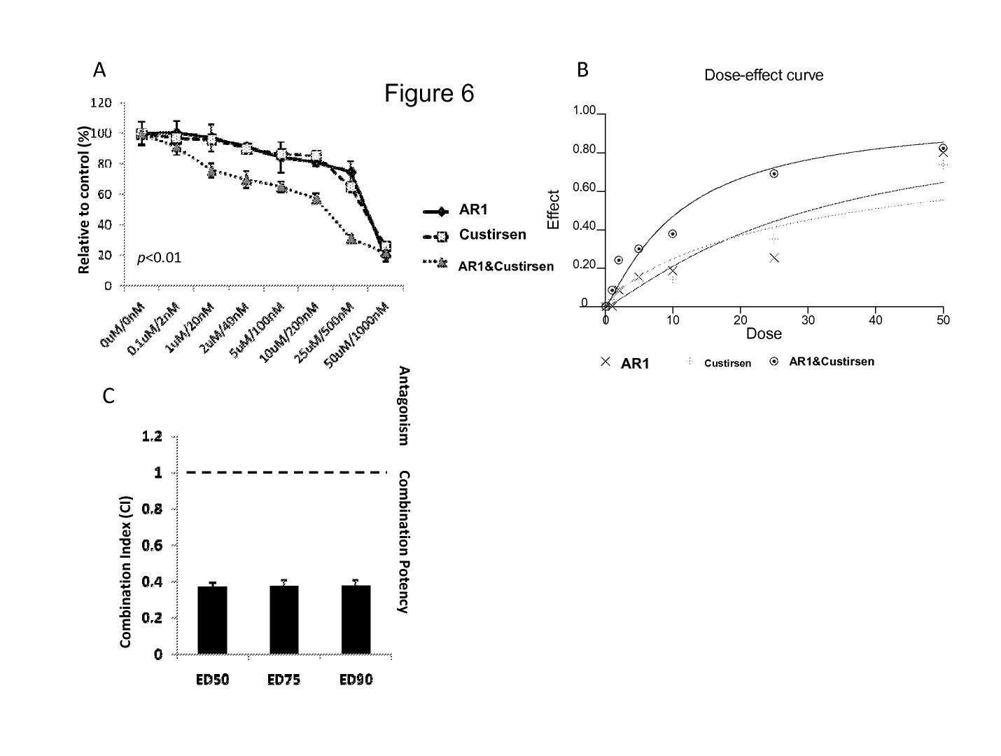

Whether CLU knockdown potentiates the anti-cancer activity of AR1 was evaluated, because anti-AR drugs (July et al., 2002) like AR1 (FIGS. 30 and 41) induce up-regulation of CLU and CLU functions as a mediator in treatment resistance (Zoubeidi et al., 2010b; Gleave et al., 2005). LNCaP cells were treated with custirsen and subsequently treated with indicated concentrations of AR1. Custirsen significantly enhanced AR1 activity, reducing cell viability compared with control ScrB plus AR1 in both time- (FIG. 43A left panel) and dose- (FIG. 43A right panel) dependent manners. To determine whether this effect was additive or a combination effect, the dose-dependent effects with constant ratio design and the CI values were calculated according to the Chou and Talalay median effect principal (Chou et al., 1984). FIG. 6B shows the dose-response curve (combination or monotherapy with custirsen or AR1) along side the CI plots, indicating that the combination of custirsen with AR1 has enhanced effects on tumor cell growth (FIG. 6C, right panel). The combination of custirsen and AR1 also had increased efficacy at reducing viability of AR positive castrate resistant C4-2 and custirsen-resistant cells compared to AR1 or custirsen monotherapy, but not in AR negative PC3 cells.

Flow cytometric analysis indicates custirsen significantly increases (P<0.001) AR1 induced apoptosis (sub-G₁ fraction) when combined with AR1 (30%) compared with control ScrB (15.2%), custirsen (20%), control ScrB+AR1 (18.3%) (FIG. 43C). In addition, combination custirsen plus AR1 increases caspase-dependent apoptosis compared with AR1 or custirsen monotherapy, as shown by cleaved PARP and caspase-3 activity. Collectively, these data indicate that the combination of custirsen and AR1 induced apoptosis more than custirsen or AR1 monotherapy.

### Example 8

### Clusterin Knock Down Combined with AR1 Treatment Mostly Enhances Cell Growth Inhibition and Apoptosis in AR Positive LNCaP Cells

LNCaP cells are seeded in 12-well culture plates in 5×10⁴ cells per well with 5% FBS or 5% CSS containing RPMI medium. The next day, cells are transfected with 10 nmol/L of CLU siRNA or SCR siRNA control at once and also daily with 500 nmol/L of custirsen or SCRB control for 2 days. The next day post transfect with siRNA or antisense oligo, LNCaP cells are treated with 10 μmol/L of AR1 and cell growth assays are performed on day 0, 1, 2, 3, 4 by crystal violet assay. (day of AR1 treatment defined as 100%). CLU knockdown+AR1 combination treatment most significantly repress cell growth. 10 μmol/L of AR1 is combined with 10 nmol/L of CLU siRNA; 10 μmol/L of AR1 combined with 500 nmol/L of custirsen.

Combination treatment enhances LNCaP apoptosis in flow cytometry analysis. Cells are treated with 10 nmol/L of CLU siRNA or SCR siRNA control at once and also daily with 500 nmol/L of custirsen or SCRB control for 2 days in 5% FBS containing RPMI medium. The next day post transfect with siRNA or antisense oligo, LNCaP cells are treated with 10 μmol/L of AR1 and FACS analysis are performed after 48 hrs treatment. The proportion of cells in sub-G0, G0-G1, S, G2-M is determined by propidium iodide staining. Combination treatment increases Sub-G0/1 apoptotic fraction apoptosis in LNCaP cells. p<0.01 in combination CLU siRNA with AR1 and p<0.001 in combination custirsen with AR1 relative to oligofectamime and DMSO control. P value represents between treatment arms and their respective controls *p<0.05, **p<0.01, ***p<0.001 (Wilcoxon matched-pairs test).

Combination treatment enhances apoptosis. LNCaP cells are pretreated with 10 μmol/L of AR1 for 48 h before treatment with CLU or SCR siRNA and custirsen or SCRB control. PARP cleavage expression levels are measured by Western blot. All experiments are repeated at least thrice.

### Example 9

### The Combination of Custirsen with AR1 Treatment Shows Increased Efficacy Compared to Custirsen or AR1 Monotherapy

Inhibition of growth is observed in LNCaP cells treated with CLU siRNA or custirsen combined with AR1 in vitro. Cells are transfected with 10 nmol/L of CLU siRNA or SCR siRNA control at once and also daily with 500 nmol/L of custirsen or SCRB control for 2 days in 5% FBS RPMI medium. The next day post transfect with siRNA or antisense oligo, LNCaP cells are treated with various concentrations of AR1. Three days after treatment, cell viability is determined by crystal violet assay. Viable cell density is normalized to that of cells treated at DMSO control (AR1 compound is dissolved in DMSO and adjusted indicated concentrations). The combination of custirsen with AR1 shows increased efficacy at reducing cell viability compared to custirsen or AR1 monotherapy. Data points are means of triplicate analysis. P value represents between treatment arms and their respective controls *p<0.05, **p<0.01 (Student's t-test).

Cell growth inhibition is evaluated for each single drug or their combination by crystal violet assay. LNCaP cells are treated with variable concentration of AR1 or custirsen. There is a significant difference between each single treatment and those combinations. P value is calculated by Friedman test. The data are input and calculated by CalcuSyn Software®. Bottom, Combination index (CI) at several effective dose. CI=1; additive effect, CI<1; combination effect, CI>1; antagonistic effect. These data indicate the combination effect at combination treatment.

### Example 10

### AR and PSA Expression in Combination Treatment

AR protein expression decreases after CLU knockdown using custirsen combined with AR1. LNCaP Cells are transfected with 10 nmol/L of CLU siRNA or SCR siRNA control at once and also daily with 500 nmol/L of custirsen or SCRB control for 2 days in 5% FBS RPMI medium. The next day post transfect with siRNA or antisense oligo, LNCaP cells are treated with 10 μmol/L of AR1. 48 hrs later, cells are harvested for protein and mRNA. The protein expression is analyzed by western blot. CLU knockdown combined with AR1 treatment has enhanced potency in decreasing AR expression compared to monotherapy. AR expression is strongly repressed by CLU knockdown with AR1. Cells are treated with 10 nmol/L of CLU siRNA or SCR siRNA combined with 10 μmol/L of AR1 or bicalutamide. AR expression is detected by western blot. Combination treatment does not affect AR mRNA level. mRNA expression is analyzed by quantitative RT-PCR, AR and PSA levels are normalized to levels of β-actin mRNA and expressed as mean±SE. **P<0.01 ***p<0.001 (Wilcoxon matched-pairs test). “OTR” means cells treated with oligofectamime only. OTR and DMSO treated cells were defined as 100%.

### Example 11

### Combination AR1 Plus Custirsen has Increased Efficacy in Delaying CRPC LNCaP Tumor Growth

The in vivo effects of co-targeting the AR and the stress response using combined custirsen with AR1 were evaluated. Male nude mice bearing LNCaP xenografts were castrated when serum PSA reached 75 ng/ml and followed until serum PSA and tumor growth rates increased back to pre-castrate levels, indicating progression to castration resistance. Mice were then randomly assigned for treatment with AR1 plus either control ScrB (n=10) or custirsen (n=10). At baseline, mean LNCaP tumor volume and serum PSA levels were similar in both groups. Custirsen significantly enhanced the antitumor effect of AR1, reducing mean tumor volume from 1600 mm³ to 650 mm³ by 12 weeks (**; p≦0.05), compared to control ScrB (FIG. 44A). Overall survival (defined as euthanasia for tumour volume exceeding 10% of body weight) was significantly prolonged with combined AR1+custirsen compared with AR1+ScrB control (90% vs 30% at week 16, respectively; *; p<0.05). Serum PSA levels were also significantly lower (˜4-folds) (FIG. 44C), and PSA doubling time is significantly prolonged (*, p<0.05) in the custirsen+AR1 group (***, p<0.001) compared with AR1 control group. To evaluate the pharmacodynamics effects of combination treatment on target protein levels, western blot analysis from tumour tissue (3 animals each) was performed for AR, PSA and CLU. FIG. 44D illustrates that AR and CLU expression levels were reduced in combination-treatment tumour tissue compared to AR1 controls. Collectively, these data demonstrate that co-targeting the AR and the resultant CLU-regulated stress response potentiates the effects of AR1 in a human CRPC xenograft model.

The efficacy of custirsen and AR1 combination therapy is enhanced compared to AR1 or custirsen monotherapy in a CRPC xenograft model. FIG. 44A-B shows the effect on tumor volume and serum PSA level by combination treatment.

### Example 12

### Combination AR1 Plus Custirsen has Increased Efficacy in CRPC Xenograft Model

LNCaP cells are inoculated s.c. into athymic nude mice. When xenografts grow to ˜500 mm³, or PSA >50 ng/ml mice are castrated. Treatment is started when PSA levels increased to pre-castration levels. Custirsen or SCRB are injected i.p. once daily for 1 week and then 3 times/week thereafter. AR1 is administrated once daily. Total LNCaP xenograft proteins are extracted in RIPA buffer after custirsen or SCRB treatment combined with AR1. (three mice per group) and Western blots are done with AR, PSA, and CLU antibodies; vinculin is used as a loading control. AR/tubulin ratio is calculated. Combination AR1 plus custirsen has increased efficacy at prolonging survival in the CRPC xenograft model.

### Example 13

### Combination AR1 Plus CLU Silencing Reduces AR Nuclear Translocation and Transcriptional Activity more Effectively than AR1 or CLU Silencing Monotherapy

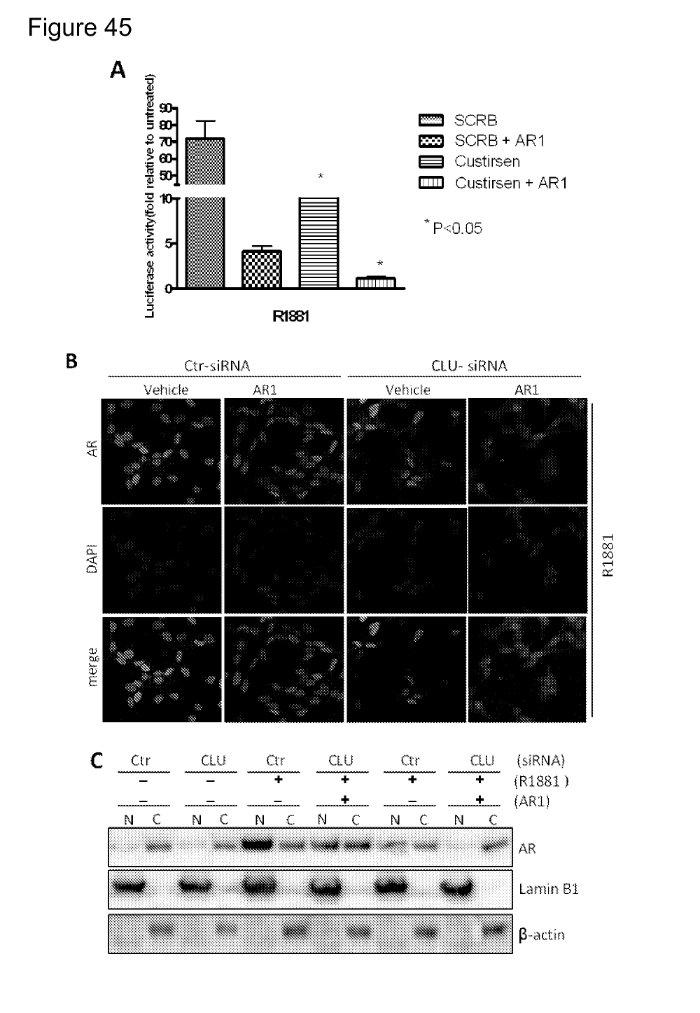

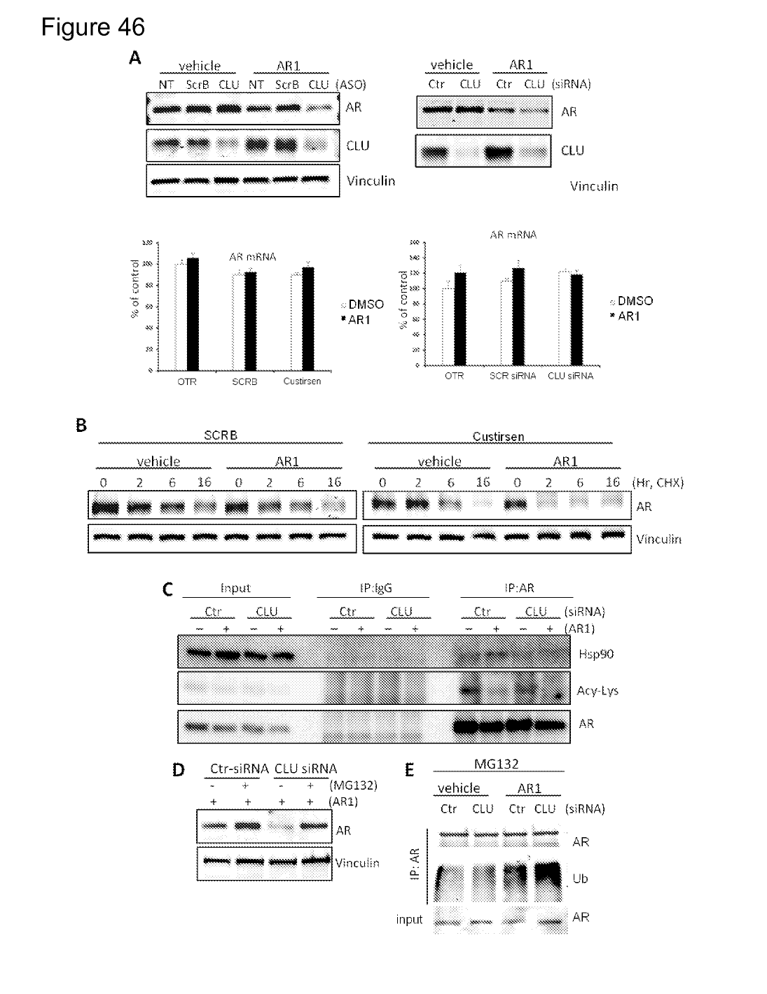

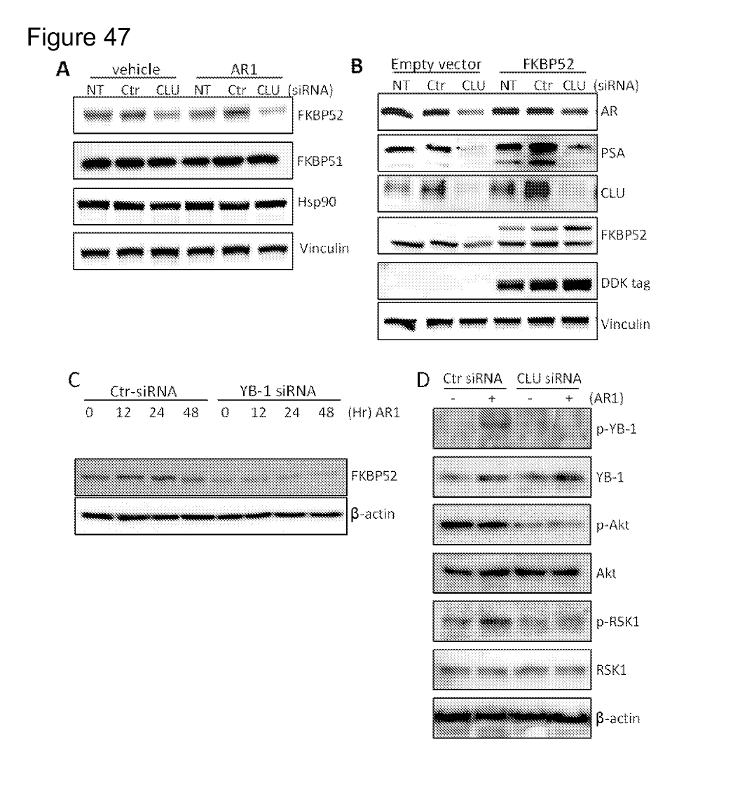

The in vivo study in FIG. 44 shows that custirsen in combination with AR1 induces rapid decrease in PSA before changes in tumor volume became apparent; in addition, AR protein levels appeared lower in the combination treated tumors compared to AR1 alone treated tumors, suggesting that CLU knockdown might potentiate AR targeting and modulate AR signalling pathway. The effects of combination treatment on androgen-induced, AR-mediated gene activation were evaluated. LNCaP cells were treated with AR1 or custirsen alone or in combination and evaluated for changes in R1881 stimulated PSA transactivation (FIG. 45A). As expected, AR1 reduced R1881 induced AR transcriptional activity, as measured by PSA luciferase transactivation assay, by 95%; interestingly custirsen also reduced AR activity by 90%, and this effect was enhanced in combination with AR1 suggesting that CLU knockdown potentiates AR1 inhibition of AR activity. To define how CLU can affect AR transcriptional activity, the effect of CLU knockdown ±AR1 on ligand-induced AR nuclear translocation was evaluated. As expected, while AR nuclear translocation was decreased by AR1, CLU knockdown in combination with AR1 maximally inhibited R1881-induced AR nuclear translocation (FIG. 45B). This co-targeting inhibitory effect was also confirmed by fractionation assay showing that targeting CLU in combination with AR1 inhibits R1881-induced nuclear AR levels (FIG. 45C).

### Example 14

### CLU Knockdown Combined with AR1 Treatment Accelerates AR Degradation Via the Proteasome Pathway.

To investigate the fate of AR after combination treatment, the effect of CLU knockdown combined with AR1 on AR expression was evaluated both at the protein and mRNA levels. CLU silencing using siRNA (FIG. 46 A right upper panel) or custirsen (FIG. 46 A left upper panel) resulted in decreased AR protein, but not mRNA levels (FIG. 46 lower panel) only in combination with AR1, suggesting that CLU knockdown in combination with AR1 may affect AR stability. AR protein stability was then evaluated using cycloheximide, which inhibits protein synthesis. AR protein levels decreased significantly with rapid degradation after custirsen-induced CLU knockdown combined with AR1 (FIG. 46B), suggesting that CLU knockdown leads to AR instability when complexed with AR1.

AR forms a heterodimer complex with Hsp90 to provide stability for ligand-unbound AR. Indeed, without Hsp90 binding, the unfolded protein will be recognized and degraded by the ubiquitin-proteasome system (Solit et al., 2003; Zoubeidi et al., 2010c). Whether AR1 affects AR binding to Hsp90, and subsequent effects if combined with CLU silencing was first evaluated. AR1 treatment actually increases AR-Hsp90 interactions, consistent with prior reports that AR1 sequesters AR in the cytoplasm. Interestingly, CLU knockdown in combination with AR1 decreases the association between AR and Hsp90, as shown in FIG. 46C. Without wishing to be bound by any scientific theory, these data are consistent with a view that AR1-AR-Hsp90 heterocomplex becomes more vulnerable to degradation under conditions of CLU silencing. To assess whether AR degradation under these conditions involves the ubiquitin-proteasome system, levels of ubiquitinated AR were measured under conditions of mono- or after combination therapy, and as shown in FIG. 46D, AR ubiquitination levels were highest under co-targeted combination conditions. AR protein levels were next evaluated in the presence or absence of proteasome inhibitor (MG132) to characterize role of proteasome and MG132 was found to abrogate AR degradation under conditions of CLU knockdown plus AR1, implicating AR degradation via the proteasome (FIG. 46E). Without wishing to be bound by any scientific theory, taken together these data suggest CLU knockdown accelerates AR degradation via a proteasome mediated pathway preferentially when the AR is bound to AR1.

### Example 15

### AR Degradation Rates are Accelerated by Combination Treatment

Combination treatment rapidly decreases AR expression. LNCaP cells are treated with 500 nmol/L of custirsen or SCRB control and then treated with 10 μmol/L of AR1 and 10 μmol/L of cycloheximide various time periods. DMSO is used as control. AR protein levels are measured by Western blot analysis. CLU knockdown combined with AR1 accelerates proteasomal degradation of AR. LNCaP cells are treated with CLU siRNA or SCR siRNA and custirsen or SCRB control, and then treated with 10 μmol/L of AR1 and 10 μmol/L MG-132 for 6 h. DMSO is used as control. AR protein levels are measured by Western blot analysis.

### Example 16

### Combination Treatment Effects AR Ubiquitination

LNCaP cells are treated with 10 nmol/L of CLU siRNA or SCR siRNA control in the presence of FBS and then treated with 10 μmol/L of AR1 and 10 μmol/L of MG-132. Immunoprecipitation is done using anti-AR antibody (N-20), and Western blot analysis is done using anti-AR antibodies (441) or anti-Ubiquitin antibodies. Input is blotted with AR (N-20) antibody. Without wishing to be bound by any scientific theory, combination treatment facilitates proteasomal degradation of AR via ubiquitination of AR.

### Example 17

### CLU Knockdown Decreases Levels of Molecular Co-Chaperones Involved in AR Stability

Without wishing to be bound by any scientific theory, the data herein indicates that AR1 monotherapy sequesters AR-Hsp90 complexes in the cytoplasm; however when combined with CLU knockdown the AR-Hsp90-AR1 heterocomplex becomes destabilized, leading to AR ubiquitination and degradation, and reduced AR nuclear transport and activity. One explanation is the CLU inhibition may lower Hsp90 levels through its affects on HSF-1 regulation (Lamoureax et al. 2011); however combination therapy did not significantly lower Hsp90 levels, and data illustrated in FIG. 42 implicates YB-1 as the key stress-activated transcription factor mediating AR1 increases in CLU. Since Hsp90 functions in cooperation with co-chaperones to confer stability of client proteins, an unbiased approach was initially used to identify Hsp90 co-chaperone affect by CLU expression. The gene profiling analysis from LNCaP cells and PC-3 treated with control and CLU siRNA disclosed herein shows that CLU expression correlated with the Hsp90 co-chaperone FKBP52 (Hsp56). Western blotting was used to confirm that CLU knockdown reduces FKBP52, but not FKBP51 or Hsp90, protein levels (FIG. 47A). To ascertain the role of FKBP52 in AR stability under conditions of AR1 treatment and CLU silencing, FKBP52 was overexpressed after CLU knockdown and AR expression was evaluated. FIG. 7B shows that FKBP52 rescues AR from degradation induced by CLU knockdown and AR1 treatment. FKBP52 overexpression also partially restores PSA expression, indicating increased AR activity when FKBP52 levels are restored under conditions of CLU knockdown. These data suggest that CLU knockdown in combination with AR1 induces AR degradation by affecting FKBP52 levels and the stability of the AR-co-chaperone complex.

To further define how CLU regulates FKBP52 levels under conditions of AR1 induced stress, public databases were mined and provided information supporting that YB-1 binds to FKBP52 with high stringency of 12 in ChIP on ChIP analysis. Western blotting confirmed that YB-1 knockdown decreases FKBP52 expression levels (FIG. 47C). AR1 treatment activates a stress response involving YB-1 transactivation of CLU, as well as increased Akt and p90rsk activity (FIG. 42). Since YB-1 knockdown decreases both CLU and FKBP52 levels, and CLU can also enhance p-AKT activity, how CLU knockdown affects interactivity between YB-1, AKT and p90rsk to affect FKBP52 levels under conditions of AR1 treatment was investigated next. Interestingly, similar to previous reports that CLU can enhance AKT phosphorylation, CLU knockdown was found to also abrogate AR1 induced phosphorylation of YB-1 and p90Rsk (FIG. 47D). Without wishing to be bound by any scientific theory, since the p90Rsk-YB-1 pathway is the key regulator for AR1 induced CLU expression (FIG. 42), collectively these data identify an AR1 treatment-induced feed forward loop involving pYB-1, p90rsk, CLU, and the AR co-chaperone FKBP52 for AR stability, nuclear translocation and activation.

### Example 18

### Combination Treatment Inhibits Akt/mTOR Signalling Pathway

AR1 activates phosphorylation of Akt and ERK. LNCaP cells are treated with AR1 in media with 5% FBS at various time periods and doses. Western blot analysis is done using phospho Akt, phospho ERK, Akt and ERK antibodies. CLU knockdown attenuates Akt/mTOR signalling pathway through inhibition of phospho Akt activation by AR1 treatment. LNCaP cells are treated with 10 nmol/L CLU siRNA or SCR siRNA control in the presence of FBS and then treated with 10 μmol/L of AR1. After 48 h treatment, western blot analysis is done using phospho Akt, phospho ERK, phospho mTOR, phospho P70S6K, Akt, ERK, mTOR and P70S6K antibodies.

### Example 19

### Possible Explanation of Combination Effect Between Clusterin Knockdown and AR1 for AR Positive State

Androgen binding to AR leads to rapid translocation from cytoplasm to nucleus and it leads to enhance activation of AR-regulated genes. Clusterin up-regulates the AKT/mTOR pathway and it leads to cell survival, cell proliferation and cell growth. Custirsen induced clusterin knockdown represses AKT phosphorylation and attenuates androgen transportation from cell surface via repressing megalin expression. AR1 strongly binds to AR and inhibits its translocation to nucleus. Without wishing to be bound by any scientific theory, these results lead to accelerate AR proteasomal degradation and down-regulated mTOR signalling pathway.

### Example 20

### Clusterin Knock Down Combined with AR1 Treatment Mostly Enhances Cell Growth Inhibition and Apoptosis in C4-2 Cells, but does not have a Combination Effect in AR Negative PC-3 Cells

C4-2 cell growth is evaluated upon combination treatment. C4-2 cells are seeded in 12-well culture plates in 3×10⁴ cells per well with 5% FBS or 5% CSS containing RPMI medium. The next day, cells are transfected with 10 nmol/L of CLU siRNA or SCR siRNA control. The next day post transfect with siRNA, C4-2 cells are treated with 10 μmol/L of AR1 and cell growth assays were performed on day 0, 1, 2, 3, 4 by crystal violet assay. (day of AR1 treatment defined as 100%). CLU knockdown and AR1 combination treatment represses cell growth most significantly. PC-3 cell growth is evaluated upon combination treatment. PC-3 cells are seeded in 12-well culture plates in 3×10⁴ cells per well with 5% FBS containing DMEM medium. The next day, cells are transfected with 10 nmol/L of CLU siRNA or SCR siRNA control at once and also daily with 500 nmol/L of custirsen or SCRB control for 2 days. The next day post transfect with siRNA or antisense oligo, PC-3 cells are treated with 10 μmol/L of AR1 and cell growth assays are performed on day 0, 1, 2, 3 by crystal violet assay. (day of AR1 treatment defined as 100%). Combination treatment enhances LNCaP apoptosis in flow cytometry analysis. Cells are treated with 10 nmol/L of CLU siRNA or SCR siRNA control at once and also daily with 500 nmol/L of custirsen or SCRB control for 2 days in 5% FBS containing RPMI medium. The next day post transfect with siRNA or antisense oligo, LNCaP cells are treated with 10 μmol/L of AR1 and FACS analysis were performed after 48 hrs treatment. Proportion of cells in sub-G0, G0-G1, S, G2-M was determined by propidium iodide staining. Combination treatment increases Sub-G0/1 apoptotic fraction apoptosis in LNCaP cells. 1Ca: combined with CLU siRNA, 1Cb: combined with custirsen.

### Example 21

### Clusterin and AR mRNA Expression is Up-Regulated in a Time and Dose Dependent Manner after AR1 Treatment

LNCaP Cells are treated with deferent time and different concentration of AR1 in RPMI1640 media with 5% FBS. AR1 is treated at various concentrations and exposure times. Cells are harvested and analyzed for mRNA level by quantitative RT-PCR. AR and CLU levels are normalized to levels of β-actin mRNA and expressed as mean±SD. AR1 exposure time of 0 h and dose of 0 μmol/L defined as 100%. *P<0.05 **P<0.01 ***p<0.001 (Wilcoxon matched-pairs test).

AR protein expression decreases after CLU knockdown combined with AR1 in both androgen depleted and androgen stimulated cells. LNCaP Cells are transfected with 10 nmol/L of CLU siRNA or SCR siRNA control in 5% CSS with or without 1 nmol/L of R1881 containing RPMI medium. The next day post transfect with siRNA, LNCaP cells are treated with 10 μmol/L of AR1. 48 hrs later, cells are harvested for protein. The protein expression is analyzed by Western blot. CLU knockdown combined with AR1 treatment decreases AR expression with greater efficacy than CLU knockdown alone or AR1 monotherapy.

### Discussion

In prostate cancer, the androgen receptor (AR) continues to drive castrate resistant progression after castration. While new AR pathway inhibitors like AR1 prolong survival in CRPC, resistance rapidly develops and is often associated with re-activation of AR signalling and induction of the cytoprotective chaperone, clusterin (CLU). Since adaptive stress pathways activated by treatment can facilitate development of acquired treatment resistance, co-targeting the stress response activated by AR inhibition, and mediated through CLU, may create conditional lethality and improve outcomes. The data herein show that co-targeted the AR and stress-induced CLU by combining AR1 with custirsen, and defined mechanisms of combination activity using the castrate-resistant LNCaP model.

AR1 induced markers of ER stress markers and chaperone proteins, including CLU, as well as the AKT and MAPK signalosome. This stress response was coordinated by a feed forward loop involving p-YB-1, p90rsk, and CLU. Combination CLU knockdown plus AR1 suppressed LNCaP cell growth rates by enhancing apoptotic rates over that seen with AR1 or custirsen monotherapy. In vivo, combined custirsen+AR1 significantly delayed castration-resistant LNCaP tumor progression and PSA progression. Mechanistically, AR1 induced AR cross talk activation of AKT and MAPK pathways was repressed with combined therapy. Interestingly, CLU knockdown also accelerated AR degradation and repressed AR transcriptional activity when combined with AR1, through mechanisms involving decreased HSF-1 and YB-1 regulated expression of AR co-chaperones FKBP52.

Co-targeting adaptive stress pathways activated by AR pathway inhibitors, and mediated through CLU, creates conditional lethality and provides mechanistic and pre-clinical proof-of-principle to guide biologically rational combinatorial clinical trial design.

Prostate cancer is the most common solid malignancy and second leading cause of cancer deaths among males in Western countries (Siegel et al., 2011). While early-stage disease is treated with curative surgery or radiotherapy, the mainstay of treatment for locally advanced, recurrent or metastatic prostate cancer is androgen ablation therapy, which reduces serum testosterone to castrate levels and suppresses androgen receptor (AR) activity. Despite high initial response rates after androgen ablation, progression to castrate resistant prostate cancer (CRPC) occurs within 3 years (Gleave et al., 2001; Bruchovsky et al., 2000; Goldenberg et al., 1999; Goldenberg et al., 1996; Gleave et al., 1998; Bruchovsky et al., 2006). Over 80% of CRPC specimens express the AR and androgen-responsive genes (Chen et al., 2004), indicating that the AR axis remains activate despite castration. Hence, the AR is a key driver of CRPC, and is supported by treatment-activated growth factor signalling pathways (Miyake et al., 2000), survival genes (Miyake et al., 1999), and cytoprotective chaperone networks (Rocchi et al., 2004). Docetaxel chemotherapy (Petrylak et al., 2004) was the first therapy to prolong survival in CRPC, stratifying the treatment landscape into pre- and post-chemotherapy states. More recently, two new AR pathway inhibitors, the CYP17 inhibitor abiraterone (de Bono et al., 2011) and the AR antagonist AR1 (Tran et al., 2009), have produced promising survival gains and are rapidly changing the CRPC landscape. Despite significant responses (Tran et al., 2009; Harris et al., 2009; Scher et al., 2010), abiraterone and AR1 activate redundant survival pathways that adaptively drive treatment resistance and recurrent CRPC progression. Realization of the full potential of these novel AR pathway inhibitors will require characterization of these stress-activated survival responses, and rational combinatorial co-targeting strategies designed to abrogate them.

Molecular chaperones play central roles in stress responses by maintaining protein homeostasis and playing prominent roles in signalling and transcriptional regulatory networks. Clusterin (CLU) is a stress-activated chaperone originally cloned as “testosterone-repressed prostate message 2” (TRPM-2) (Montpetit et al., 1986) from post-castration regressing rat prostate, but was subsequently defined as a stress-activated and apoptosis-associated, rather than an androgen-repressed, gene (Cochrane et al., 2007). CLU is transcriptionally regulated by HSF1 (Lamoureux et al., 2011) and YB-1 (Shiota et al., 2011), inhibiting stress-induced apoptosis by suppressing protein aggregation (Poon et al., 2002), p53-activating stress signals (Trougakos et al., 2009), and conformationally-altered Bax (Zhang et al., 2005; Trougakos et al., 2009) while enhancing Akt phosphorylation (Ammar et al., 2008) and trans-activation of NF-κB and HSF-1 (Lamoureux et al., 2011; Shiota et al., 2011; Poon et al., 2002; Trougakos et al., 2009; Zhang et al., 2005; Ammar et al., 2008; Zoubeidi et al., 2010a). CLU is expressed in many human cancers (Yom et al., 2009; Kruger et al., 2007; Zhang et al., 2006), including prostate, where it increases following castration and to become highly expressed in CRPC (July et al., 2002). CLU over-expression confers treatment resistance (Miyake et al., 2000), while CLU inhibition potentiates activity of most anti-cancer therapies in many preclinical models (Miyake et al., 2005; Sowery et al., 2008; Gleave et al., 2005; Zoubeidi et al., 2010b). The CLU inhibitor, OGX-011 (custirsen, OncoGenex Pharmaceuticals), is currently in Phase III trials after a randomized phase II study in CRPC reported 7 month gain in overall survival and 50% reduced death rate (HR=0.50) when combined with docetaxel chemotherapy (Chi et al., 2010).

Since CLU is induced by treatment stress, including castration, and functions as an important mediator of the stress response, the hypothesis herein that AR1 treatment induces the stress response and CLU, and that co-targeting the AR and stress-response pathways mediated by CLU may create conditional lethality and improve cancer control was tested. The data described herein set out to correlate AR1 treatment stress and resistance with CLU induction, identify pathways regulating CLU activation, and define mechanisms by which CLU inhibition potentiates anti-AR therapy in CRPC.

Many strategies used to kill cancer cells induce stress- and redundant survival responses that promote survival and emergence of treatment resistance, which is the underlying basis for most cancer deaths. This therapeutic resistance results from a Darwinian interplay of innate and adaptive survival pathways activated by selective pressures of treatment. In prostate cancer, androgen ablation induces tumor cell apoptosis and clinical responses in most patients but also triggers progression within 2-3 years to castration resistant prostate cancer (CRPC) (Gleave et al., 2001; Bruchovsky et al., 2000; Goldenberg et al., 1999; Goldenberg et al., 1996; Gleave et al., 1998). Experimentally, CRPC progression is attributed to re-activation of the AR axis (Miyake et al., 2000; Miyake et al., 1999) supported by growth factor (Miyake et al., 2000; Culig et al., 2004; Craft et al., 1999) and survival gene (Miyake et al., 1999; Gleave et al., 1999; Miayake et al., 2000; Rocchi et al., 2004; Miyake et al., 2000) networks. Recently new AR pathway inhibitors like abiraterone and AR1 (Rocchi et al., 2004) have been shown to prolong survival and clinically validate the AR as the main driver of CRPC (Miyake et al., 2000; Miyake et al., 1999). Not all patients respond to these inhibitors, and resistance develops in many initial responders (Petrylak et al., 2004); moreover, disease progression frequently correlates with a rising PSA level, indicating continued AR signalling and highlighting need for additional therapies targeting the molecular basis of treatment resistance in CRPC. Defining interactions between the AR and redundant survival pathways will build new combinatorial strategies that control progression and improve outcomes.

Persistent AR signalling in CRPC is postulated to occur via AR amplification and mutations that increase sensitivity to low levels of DHT and other steroids (Miyake et al., 2000; Miyake et al., 1999; Zoubeidi et al., 2007), or AR splice variants that drive constitutively active truncated receptors lacking a LBD (Nizard et al., 2007; Carver et al., 2011; Evdokimova et al., 2006). Other AR-related mechanisms include altered levels of AR coactivators or co chaperones (hsp27), and AR phosphorylation via activated src or tyrosine kinase receptors like EGFR (Chi et al., 2010). Another more dynamic mechanism involves reciprocal feedback regulation between AR and PI3K pathways whereby AR inhibition activates AKT signaling by reducing levels of the AKT phosphatase PHLPP, and PI3K inhibition activates AR signaling by relieving feedback inhibition of HER kinases; inhibition of one activates the other, thereby enhancing survival. These mechanistic insights are guiding design of combinatorial regimens co-targeting the AR pathway with inhibitors against histone deacetylase (de Bono et al., 2011), src, AKT, and AR chaperone heat-shock proteins (Hsp)-90 and Hsp27.

Inhibiting the stress response activated by AR pathway inhibitors is another combinatorial co-targeting strategy. Many anti-cancer agents induce ER stress (Rutkowski et al., 2007), which activates a complex intracellular signaling pathway, termed the unfolded protein response (UPR), tailored to reestablish protein homeostasis by inhibiting protein translation and stimulating the ubiquitin-proteasome system (UPS) to enhance ER-associated protein degradation (ERAD) (Harding et al., 2002). Chaperones like CLU are key mediators of ER stress responses. AR pathway inhibition is known to induce ER stress and CLU with reciprocal pathway activation of AKT, which are all implicated in castration resistance. Consistent with these prior reports, the data herein show that AR1 induces ER stress and the UPR, and go on to define feed-forward links between stress-induced YB-1 activity to CLU activation in parallel with enhanced AKT and MAPK signaling, which collectively support AR stability and activity under AR1 treatment conditions. YB-1 and CLU are both stress-activated survival chaperone proteins functionally associated with anti-cancer treatment resistance (Poon et al., 2002) (Zoubeidi et al., 2007). Under stress conditions, YB-1 is phospho-activated by AKT (Evdokimova et al., 2006) and p90RSK (cleave et al., 2005), stimulating its nuclear translocation and binding to target promoters. CLU is transcribed by, and acts as, a critical downstream mediator of stress-induced YB-1 activity and paclitaxel resistance (Shiota). YB-1 can also function as an mRNA chaperone protein to regulate translation of certain stress-associated transcripts (Law et al., 2010; Evdokimova et al., 2009). Using YB1 RNA-IP hybridized to microarrays with different platform technologies, YB1 was found to bind to CLU mRNA. The data disclosed herein show that YB1 binds preferentially to CLU mRNA after AR1 induced ER stress, and found that YB1 is associated with CLU-mRNA in different polysomal fraction. Since polysomal fractions represent translationally active mRNAs that are bound by ribosomes or other elements of the translational machinery, and post-polysomal mRNAs are ribosome-depleted and hence translationally inactive; Evdokimova et al., 2009; Evdokimova et al., 2006a), CLU mRNA will be amplified from these fractions. These data indicate that YB-1 mediates not only transcriptional, but also translational, induction of CLU in response to AR1 induced ER stress.

CLU is a stress-activated molecular chaperone closely linked to treatment resistance and cancer progression (Miyake et al., 2000; Gleave and Miyake, 2005; Trougakos and Gonos, 2009b), where its overexpression confers broad-spectrum treatment resistance (Tran et al., 2009; Yom et al., 2009). Similar to castration and other treatment stressors, AR1 increases CLU expression levels; moreover, CLU levels are higher in AR1 resistant tumors, as they are in CRPC compared to castrate naïve cancers. CLU is not only transcriptionally regulated by HSF-1, but also enhances HSF-1-mediated transcriptional activity in a feed-forward manner (Lamoureax et al., 2011). CLU is also activated by prosurvival pathways including the AR and downstream of IL-6 (via JAK/stat) and IGF-1R (via Src-MEK-ERK-Erg-1) signalling pathways. CLU suppresses stress-induced apoptosis by inhibiting protein aggregation, p53-activating stress signals, and conformationally-altered Bax (Zhang et al., 2005; Trougakos et al., 2009) while enhancing Akt phosphorylation (Sowery et al., 2008; Chou et al., 1984) and trans-activation of NF-κB and HSF-1.

This stress-activated anti-apoptotic function for CLU results in broad-spectrum resistance to many anti-cancer therapies, and identifies it as a potential anti-cancer target. A CLU antisense inhibitor, custirsen, enhances cancer cell death in combination with therapeutic stressors in many preclinical cancer models. Indeed, combination docetaxel plus custirsen phase III clinical trials are underway in CRPC after randomized Phase II studies reported a significant survival benefit when custirsen was added to docetaxel (Zoubeidi et al., 2010b; Culig et al., 2004). While CLU inhibition has been reported to enhance castration and delay time to CRPC in androgen-dependent xenografts, the pure AR antagonist AR1 now enables investigation of effects of AR pathway inhibition and CLU in treatment response in vitro and in vivo in CRPC models. The data disclosed herein established that CLU was induced after AR1 and AR knockdown in AR1-sensitive and resistant LNCaP cells, respectively, and that CLU remained highly expressed in most AR1 resistant LNCaP xenografts and cell lines. Co-targeting the AR and CLU using AR1 plus custirsen enhanced apoptotic rates over monotherapy. Mechanistically, AR1 induced cross talk activation of AKT and MAPK pathways was repressed with combined therapy. Unexpectedly, we found that AR ubiquitination and proteasome-mediated degradation rates were accelerated when AR1 was combined with CLU knockdown. While AR1 alone did not alter AR stability, when CLU was inhibited, stress-activation of YB-1 and MAPK was blunted, resulting in decreased YB-1 activated expression of AR co-chaperones Hsp56 (FKBP52) and Hsp90, which led to ubiquitination and proteasomal degradation of the AR, decreasing AR transcriptional activity beyond that observed with AR1 monotherapy, and even in AR1 resistant cell lines.

These results highlight a role for CLU in supporting YB-1 mediated expression of other molecular chaperones under context dependent stress conditions, similar to its ability to enhance HSF-1-mediated transactivation of Hsp70 and Hsp27 after Hsp90 inhibition (Lamoureax et al., 2011). In addition to CLU, HSF-1 and YB-1 orchestrate expression of other molecular chaperones involved in processes of folding, trafficking, and transcriptional activation of the AR and other steroid receptors. In the absence of ligand, AR is predominately cytoplasmic, maintained in an inactive, but highly responsive state by a large dynamic heterocomplex composed of Hsp90 and Hsp70, and co-chaperones like Hsp56. These Hsp AR co-chaperones play important roles in AR stability and activation. (Cheung-Flynn et al., 2005; Yang et al., 2006). Ligand binding leads to a conformational change in the AR and dissociation from the large Hsp complex to associate with Hsp27 for nuclear transport and transcriptional activation of target genes (Zoubeidi et al., 2006; Abdul et al., 2001). Compared to the first generation AR antagonist bicalutamide, which does not inhibit AR nuclear transport, we show that AR1-bound AR remains cytoplasmic and complexed with its Hsp chaperones, Hsp90 and FKBP52. This cytoplasmic confinement of AR complexed with its Hsp co-chaperones may increase its susceptibility to degradation under conditions of ER stress and chaperone suppression.

Without wishing to be bound by any scientific theory, the data herein identifies another mechanism by which CLU inhibition potentiates anti-AR therapy, via suppression of MAPK and Akt signalling pathways activated after AR pathway inhibition. The data herein confirm previous reports that AR1 induces Akt phosphorylation, and also show that MAPK and p90rsk are activated by AR1 to mediate YB-1 phosphorylation. The results herein demonstrate that CLU knock down combined with AR1 abrogates both Akt and p90rsk activation.

Without wishing to be bound by any scientific theory, the data herein define a stress-induced feed-forward loop involving AR1-induced YB-1 transactivation of CLU, with CLU facilitating pro-survival AKT and p90rsk signalling, phospho-activation of YB-1, and expression of AR co-chaperones that stabilize the AR under conditions of AR1 treatment. Co-targeting adaptive stress pathways activated by AR pathway inhibitors, and mediated through CLU, potentiate anti-AR activity by decreasing AR expression levels and activity, as well as activation of Akt and MAPK signaling pathways induced by AR1. These results provide mechanistic and pre-clinical proof-of-principle to support combinatorial clinical studies with AR1 and custirsen.

Aspects of the present invention relate to the unexpected discovery that an oligonucleotide targeting clusterin expression such as custirsen, together with an AR antagonist as a combination is more potent than a monotherapy of either agent for treatment of prostate cancer. This increased efficacy is in addition to increased cancer cell death, and includes reduced proliferation of the cancer cells, reduced translocation of AR from the cytoplasm to the nucleus, reduced transcriptional activity of AR, increased PARP cleavage, reduced AKT phosphorylation, reduced ERK phosphorylation, and increased AR protein degradation. FIG. 30 shows that AR1 resistant prostate cancer tumors have increased clusterin expression. Without wishing to be bound by any scientific theory, the data herein may be reflecting that the resistance of these tumors to AR1 may be due to increased clusterin expression, and therefore, decreasing clusterin expression increases the sensitivity of AR1 resistant tumors to AR1 treatment. Thus, AR1 resistant prostate cancer cells are sensitized to AR1 by concomitant treatment with custirsen.

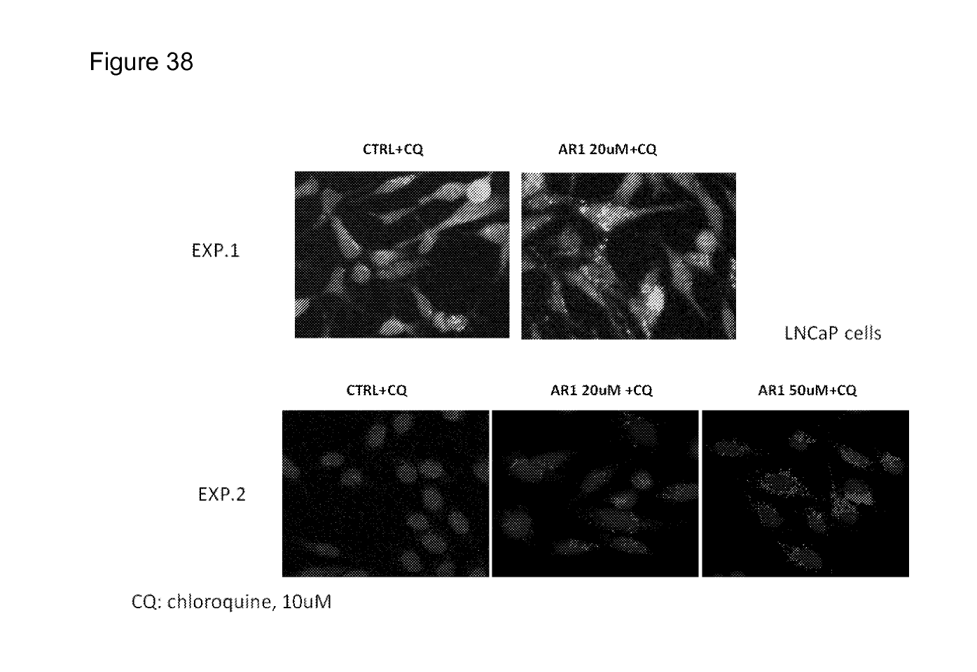

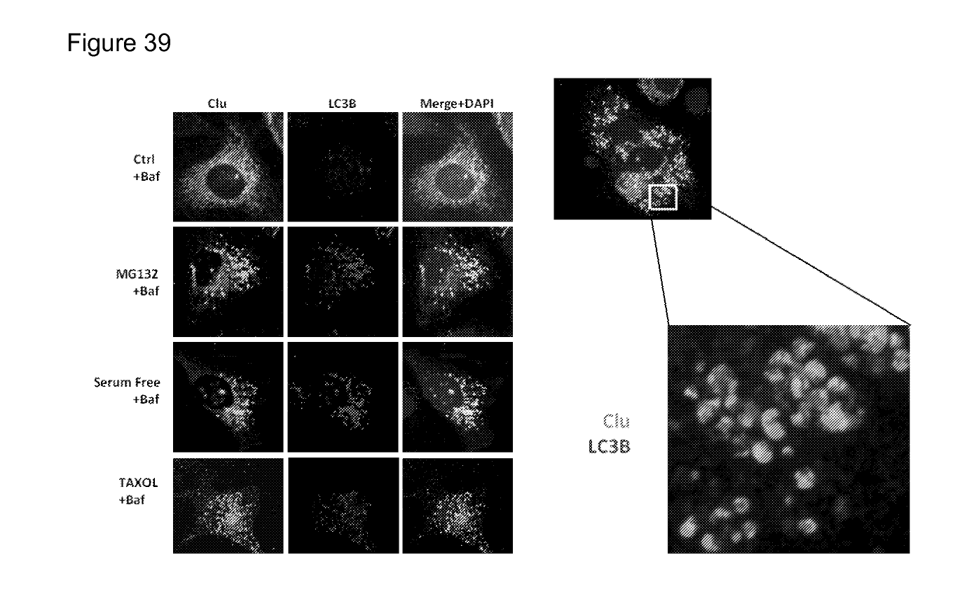

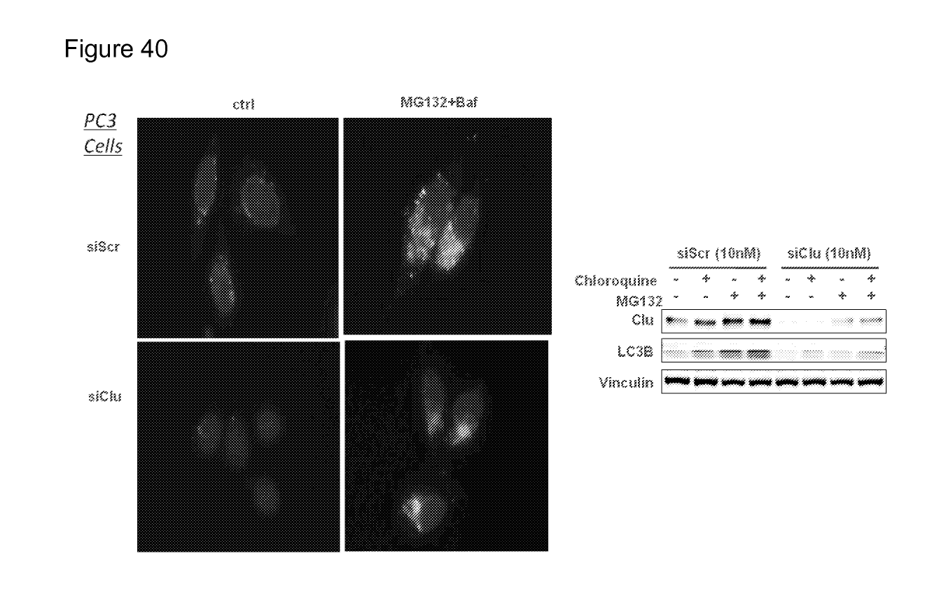

AR1 induces autophagy in prostate cancer cells (FIG. 38), and clusterin silencing can inhibit ER stress-induced autophagy. Autophagy is a well conserved lysosomal degradation pathway for intra-cellular digestion that can confer stress tolerance and sustain cell viability under adverse conditions. Without wishing to be bound by any scientific theory, it is possible that increased autophagy following AR1 treatment may enhance prostate cancer cell survival, and inhibition of clusterin expression inhibits this increased autophagy, thereby resulting in reduced cancer cell survival and enhanced AR1 activity.

Without wishing to be bound by any scientific theory, decreased clusterin expression may enhance the activity of AR1 by decreasing AR stability via suppression of HSF-1 mediated regulation of AR co-chaperones such as FKBP52 and Hsp27.

Without wishing to be bound by any scientific theory, decreased clusterin expression may enhance the activity of AR1 by decreasing the induction of AKT levels and/or phosphorylation following AR1 treatment.

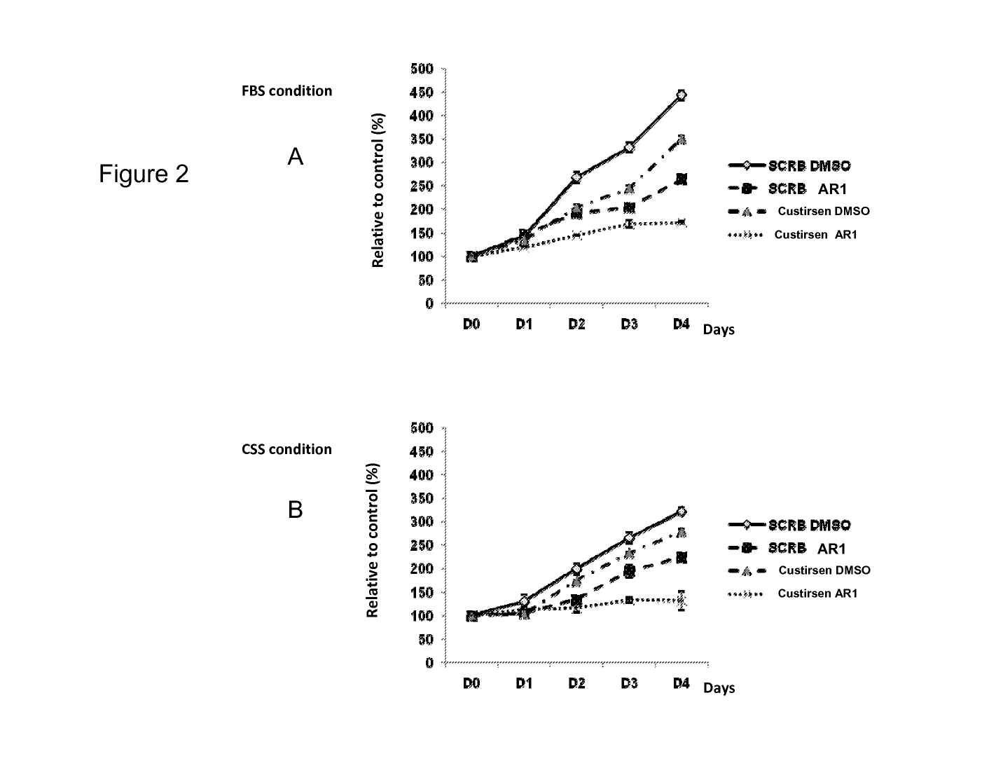

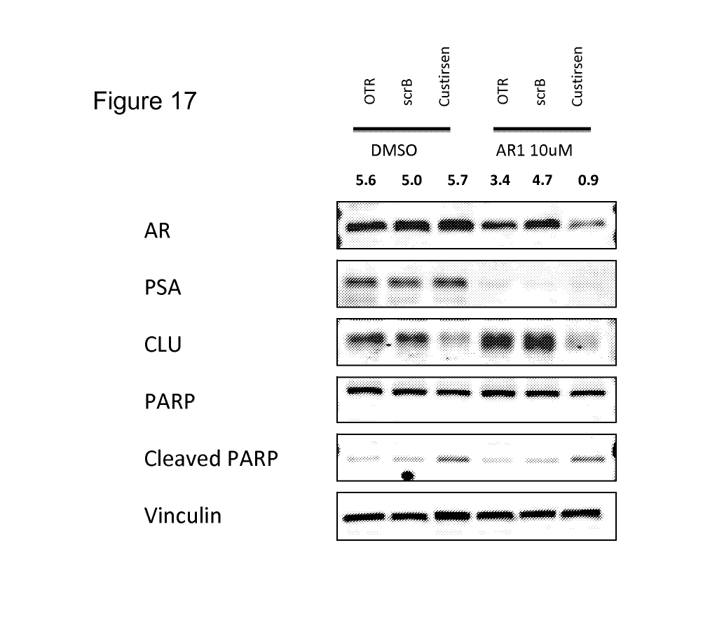

AR1 monotherapy is able to treat castration-resistant prostate cancer in humans; however, custirsen monotherapy has not been shown to inhibit the progression of prostate cancer after it has progressed to androgen-independence in any model system. It is therefore surprising that the combination treatment of AR1 and custirsen would be more potent than treatment with AR1 alone. Furthermore, AR1 and custirsen combination therapy is surprisingly potent, and is able to halt prostate cancer cell growth in vitro, whereas cells receiving either agent alone proliferate by over 200% over a period of four days (FIG. 2B). Surprisingly, also, the combination of custirsen and AR1 is able to reduce AR protein expression by over 80%, whereas custirsen alone has no effect, and AR1 alone reduces expression by only about 40% (FIG. 17). Finally, the combination of custirsen and AR1 increases the survival of treated mice afflicted with castration-resistant prostate cancer to about 90% at 16 weeks from the start of treatment, as compared to about 40% for AR1 alone.

Additionally, the combination of AR1 and custirsen is more effective at reducing tumor growth in mammals than the combination of bicalutamide and custirsen. The combination of AR1 and custirsen is more effective at reducing tumor growth in mammals than the combination of flutamide and custirsen. The combination of AR1 and custirsen is more effective at reducing cancer cell proliferation than the combination of bicalutamide and custirsen. The combination of AR1 and custirsen is more effective at reducing cancer cell proliferation than the combination of flutamide and custirsen.

The combination of AR1 and custirsen is more effective at increasing cancer cell apoptosis than the combination of bicalutamide and custirsen. The combination of AR1 and custirsen is more effective at increasing cancer cell apoptosis than the combination of flutamide and custirsen.

In addition to increased anti-tumor potency, combination therapy may also allow dose reduction strategies to reduce toxicity. For example, AR1 is known to induce side effects such as fatigue and has a maximum tolerated dose of 240 mg/day (Scher et al., 2010). However, the present invention discloses that doses of AR1 as low as 10 mg/kg/day in combination with custirsen are effective to decrease tumor size and prolong survival in mice. The NIH provides guidance on the conversion of doses used in mouse studies to those appropriate for human use based on Equivalent Surface Area Dosage Conversion Factors (NIH Equivalent Surface Area Dosage Conversion Factors Guidance, Posted August 2007; Freidreich et al., 1966). According to the NIH conversion factor table, the 10 mg/kg/day dose described for use in mice herein is equivalent to 0.83 mg/kg/day in a 60 kg human, equaling a dose of about 49.8 mg/day for a 60 kg human, or about 83 mg/day for a 100 kg human. These doses are much lower than the dose of 240 mg/kg recommended for phase III trials in humans (Scher et al., 2009). Therefore, an aspect of the invention provides a combination of an anti-clusterin oligonucleotide and an AR antagonist effective to treat prostate cancer in which the amount of the AR antagonist in the combination is less than the effective amount used in monotherapy. The surprising potency of combination therapy comprising an oligonucleotide which reduces clusterin levels and an AR antagonist can be used to decrease doses of one or both agents in humans, enabling therapeutic benefit with less side effects.

### REFERENCES

Abdul, M. and N. Hoosein, Inhibition by anticonvulsants of prostate-specific antigen and interleukin-6 secretion by human prostate cancer cells. Anticancer Res, 2001. 21(3B): p. 2045-8.
Ammar, H. and J. L. Closset, Clusterin activates survival through the phosphatidylinositol 3-kinase/Akt pathway. J Biol Chem, 2008. 283(19): p. 12851-61.
Bruchovsky, N., et al., Characterization of 5 a-reducatase gene expression in stroma and epithelium of human prostate. Journal of Steroid Biochemistry and Molecular Biology, 1996. 59(5/6): p. 397-404.
Bruchovsky, N., et al., Intermittent androgen suppression for prostate cancer: Canadian Prospective Trial and related observations. Mol Urol, 2000. 4(3): p. 191-9; discussion 201.
Carthew, R. W., Gene silencing by double-stranded RNA. Current Opinion in Cell Biology; 2001, 13:244-248.
Carver, B. S., et al., Reciprocal feedback regulation of PI3K and androgen receptor signaling in PTEN-deficient prostate cancer. Cancer Cell, 2011. 19(5): p. 575-86.
Chen, C. D., et al., Molecular determinants of resistance to antiandrogen therapy. Nat Med, 2004. 10(1): p. 33-9.
Cheung-Flynn, J., et al., Physiological role for the cochaperone FKBP52 in androgen receptor signaling. Mol Endocrinol, 2005. 19(6): p. 1654-66.
Chi et al., A Phase I Pharmacokinetic and Pharmacodynamic Study of custirsen, a 2′-Methoxyethyl antisense Oligonucleotide to Clusterin, in Patients with Localized Prostate Cancer. Journal of the National Cancer Institute; 2005, 97(17)1287-1296.
Chi et al., A phase I pharmacokinetic (PK) and pharmacodynamic (PD) study of custirsen, a 2′ methoxyethyl phosphorothioate antisense to clusterin, in patients with prostate cancer prior to radical prostatectomy. Journal of Clinical Oncology; 2004 ASCO Annual Meeting Proceedings, vol. 22, no. 14S:3033.
Chi, K. N., et al., Randomized phase II study of docetaxel and prednisone with or without OGX-011 in patients with metastatic castration-resistant prostate cancer. J Clin Oncol, 2010. 28(27): p. 4247-54.
Chou, T. C. and P. Talalay, Quantitative analysis of dose-effect relationships: the combined effects of multiple drugs or enzyme inhibitors. Adv Enzyme Regul, 1984. 22: p. 27-55.
Cochrane, D. R., et al., Differential regulation of clusterin and its isoforms by androgens in prostate cells. J Biol Chem, 2007. 282(4): p. 2278-87.
Craft, N., et al., A mechanism for hormone-independent prostate cancer through modulation of androgen receptor signaling by the HER-2/neu tyrosine kinase. Nature Medicine, 1999. 5(3): p. 280-5.
Culig, Z., Androgen receptor cross-talk with cell signalling pathways. Growth Factors, 2004. 22(3): p. 179-84.
de Bono, J. S., et al., Abiraterone and increased survival in metastatic prostate cancer. N Engl J Med, 2011. 364(21): p. 1995-2005.
Elbashir et al., Duplexes of 21-nucleotide RNAs mediate RNA interference in cultured mammalian cells. Nature; 2001, 411:494-498.
Evdokimova, V., et al., Translational activation of snail1 and other developmentally regulated transcription factors by YB-1 promotes an epithelial-mesenchymal transition. Cancer Cell, 2009. 15(5): p. 402-15.
Evdokimova, V., et al., Akt-mediated YB-1 phosphorylation activates translation of silent mRNA species. Mol Cell Biol, 2006. 26(1): p. 277-92.
Evdokimova, V., L. P. Ovchinnikov, and P. H. Sorensen, Y-box binding protein 1: providing a new angle on translational regulation. Cell Cycle, 2006a. 5(11): p. 1143-7.
Fire et al., Potent and specific genetic interference by double-stranded RNA in Caenorhabditis elegans. Nature; 1998, 391:806-11.
Freireich, et al., Quantitative comparison of toxicity of anticancer agents in mouse, rat, dog, monkey, and man. Cancer Chemother Rep. 1966; 50(4):219-244.
Gleave, M. E., et al., Randomized comparative study of 3 versus 8-month neoadjuvant hormonal therapy before radical prostatectomy: biochemical and pathological effects. J Urol, 2001. 166(2): p. 500-6; discussion 506-7.
Gleave, M., et al., Intermittent androgen suppression for prostate cancer: rationale and clinical experience. Prostate Cancer Prostatic Dis, 1998. 1(6): p. 289-296.
Gleave, M. and H. Miyake, Use of antisense oligonucleotides targeting the cytoprotective gene, clusterin, to enhance androgen- and chemo-sensitivity in prostate cancer. World J Urol, 2005. 23(1): p. 38-46.
Gleave, M. and K. N. Chi, Knock-down of the cytoprotective gene, clusterin, to enhance hormone and chemosensitivity in prostate and other cancers. Ann N Y Acad Sci, 2005. 1058: p. 1-15.
Gleave, M., et al., Progression to androgen independence is delayed by adjuvant treatment with antisense Bcl-2 oligodeoxynucleotides after castration in the LNCaP prostate tumor model. Clin Cancer Res, 1999. 5(10): p. 2891-8.
Goldenberg, S. L., et al., Clinical Experience with Intermittent Androgen Suppression in Prostate Cancer: Minimum of 3 Years' Follow-Up. Mol Urol, 1999. 3(3): p. 287-292.
Goldenberg, S. L., et al., Low dose cyproterone acetate plus mini-dose diethylstilbesterol—a protocol for reversible medical castration. Urology, 1996. 47(6): p. 882-884.
Harding, H. P., et al., Transcriptional and translational control in the Mammalian unfolded protein response. Annu Rev Cell Dev Biol, 2002. 18: p. 575-99.
Harris, W. P., et al., Androgen deprivation therapy: progress in understanding mechanisms of resistance and optimizing androgen depletion. Nat Clin Pract Urol, 2009. 6(2): p. 76-85.
July, L. V., et al., Clusterin expression is significantly enhanced in prostate cancer cells following androgen withdrawal therapy. Prostate, 2002. 50(3): p. 179-88.
Krajewska et al., Immunohistochemical analysis of bcl-2, bax, bcl-X, and mcl-1 expression in prostate cancers. Am. J. Pathol; 1996, 148:1567-1576.
Kruger, S., et al., Prognostic significance of clusterin immunoreactivity in breast cancer. Neoplasma, 2007. 54(1): p. 46-50.
Lamoureux, F., et al., Clusterin Inhibition using OGX-011 Synergistically Enhances Hsp90 Inhibitor Activity by Suppressing the Heat Shock Response in Castrate Resistant Prostate Cancer. Cancer Res, 2011.
Lassi et al., Update on castrate-resistant prostate cancer: 2010. Current Opinion in Oncology; 2010, 22:263-267.
Law, J. H., et al., Molecular decoy to the Y-box binding protein-1 suppresses the growth of breast and prostate cancer cells whilst sparing normal cell viability. PLoS One. 5(9).
Miyaki et al., Antisense oligodeoxynucleotide therapy targeting clusterin gene for prostate cancer: Vancouver experience from discovery to clinic. International Journal of Urology; 2005, 12: 785-794.
McDonnell et al., Expression of the proto-oncogene bcl-2 in the prostate and its association with the emergence of androgen-independent prostate cancer. Cancer Res; 1992, 52:6940-6944.
Miyaki et al., Antisense oligodeoxynucleotide therapy targeting clusterin gene for prostate cancer: Vancouver experience from discovery to clinic. International Journal of Urology; 2005, 12: 785-794.
Miyake, H., et al., Overexpression of insulin-like growth factor binding protein-5 helps accelerate progression to androgen-independence in the human prostate LNCaP tumor model through activation of phosphatidylinositol 3′-kinase pathway. Endocrinology, 2000. 141(6): p. 2257-65.
Miyake, H., A. Tolcher, and M. E. Gleave, Antisense Bcl-2 oligodeoxynucleotides inhibit progression to androgen-independence after castration in the Shionogi tumor model. Cancer Res, 1999. 59(16): p. 4030-4.
Miyake, H., et al., Testosterone-repressed prostate message-2 is an antiapoptotic gene involved in progression to androgen independence in prostate cancer. Cancer Res, 2000. 60(1): p. 170-6.
Miyake, H., I. Hara, and M. E. Gleave, Antisense oligodeoxynucleotide therapy targeting clusterin gene for prostate cancer: Vancouver experience from discovery to clinic. Int J Urol, 2005. 12(9): p. 785-94.
Miayake, H., A. Tolcher, and M. E. Gleave, Chemosensitization and delayed androgen-independent recurrence of prostate cancer with the use of antisense Bcl-2 oligodeoxynucleotides. J Natl Cancer Inst, 2000. 92(1): p. 34-41.
Montpetit, M. L., K. R. Lawless, and M. Tenniswood, Androgen-repressed messages in the rat ventral prostate. Prostate, 1986. 8(1): p. 25-36.
NIH Equivalent Surface Area Dosage Conversion Factors Guidance, Posted August 2007. Accessed from web.ncifcrf.gov/rtp/lasp/intra/acuc/fred/guidelines/ACUC4-2EquivSurfAreaDosageConversion.pdf on Jan. 14, 2011.
Nizard, P., et al., Stress-induced retrotranslocation of clusterin/ApoJ into the cytosol. Traffic, 2007. 8(5): p. 554-65.
Oh et al., Management of hormone refractory prostate cancer: current standards and future prospects. J Urol; 1998, 160(4):1220-9.
Petrylak, D. P., et al., Docetaxel and estramustine compared with mitoxantrone and prednisone for advanced refractory prostate cancer. N Engl J Med, 2004. 351(15): p. 1513-20.
Poon, S., et al., Mildly acidic pH activates the extracellular molecular chaperone clusterin. J Biol Chem, 2002. 277(42): p. 39532-40.
Raffo et al., Overexpression of bcl-2 protects prostate cancer cells from apoptosis in vitro and confers resistance to androgen depletion in vivo. Cancer Res; 1995 55(19): 4448-4445.
Rocchi, P., et al., Heat shock protein 27 increases after androgen ablation and plays a cytoprotective role in hormone-refractory prostate cancer. Cancer Res, 2004. 64(18): p. 6595-602.
Rutkowski, D. T. and R. J. Kaufman, That which does not kill me makes me stronger: adapting to chronic ER stress. Trends Biochem Sci, 2007. 32(10): p. 469-76.
Scher, H. I., et al., Antitumour activity of AR1 in castration-resistant prostate cancer: a phase 1-2 study. Lancet, 2010. 375(9724): p. 1437-46.
Scher et al., Antitumor activity of MDV3100 in a phaseI/II study of castration-resistant prostate cancer (CRPC). 2009 ASCO Meeting, J Clin Oncol 27:15s, 2009 (suppl; abstr 5011).
Sensibar et al., Prevention of Cell Death Induced by Tumor Necrosis Factor α in LNCaP Cells by Overexpression of Sulfated Glycoprotein-2 (Clusterin). Cancer Research; 1995, 55: 2431-2437.
Shiota, M., et al., Clusterin is a critical downstream mediator of stress-induced YB-1 transactivation in prostate cancer. Mol Cancer Res, 2011.
Siegel, R., et al., Cancer statistics, 2011: the impact of eliminating socioeconomic and racial disparities on premature cancer deaths. CA Cancer J Clin, 2011. 61(4): p. 212-36.
Solit, D. B., H. I. Scher, and N. Rosen, Hsp90 as a therapeutic target in prostate cancer. Semin Oncol, 2003. 30(5): p. 709-16.
Sowery, R. D., et al., Clusterin knockdown using the antisense oligonucleotide OGX-011 re-sensitizes docetaxel-refractory prostate cancer PC-3 cells to chemotherapy. BJU Int, 2008. 102(3): p. 389-97.
Stratford, A. L., et al., Y-box binding protein-1 serine 102 is a downstream target of p90 ribosomal S6 kinase in basal-like breast cancer cells. Breast Cancer Res, 2008. 10(6): p. R99.
Tran, C., et al., Development of a second-generation antiandrogen for treatment of advanced prostate cancer. Science, 2009. 324(5928): p. 787-90.
Trougakos, I. P., et al., Intracellular clusterin inhibits mitochondrial apoptosis by suppressing p53-activating stress signals and stabilizing the cytosolic Ku70-Bax protein complex. Clin Cancer Res, 2009. 15(1): p. 48-59.
Wong et al., Molecular characterization of human TRPM-2/clusterin, a gene associated with sperm maturation, apoptosis and neurodegeneration. Eur. J. Biochem. 1994, 221 (3):917-925.
Yagoda et al., Cytotoxic chemotherapy for advanced hormone-resistant prostate cancer. Cancer; 1993, 71 (Supp. 3): 10981109.
Yang, Z., et al., FK506-binding protein 52 is essential to uterine reproductive physiology controlled by the progesterone receptor A isoform. Mol Endocrinol, 2006. 20(11): p. 2682-94.
Yom, C. K., et al., Clusterin overexpression and relapse-free survival in breast cancer. Anticancer Res, 2009. 29(10): p. 3909-12.
Zhang, H., et al., Clusterin inhibits apoptosis by interacting with activated Bax. Nat Cell Biol, 2005. 7(9): p. 909-15.
Zhang, S., et al., Clusterin expression and univariate analysis of overall survival in human breast cancer. Technol Cancer Res Treat, 2006. 5(6): p. 573-8.
Zoubeidi, A., et al., Clusterin facilitates COMMD1 and I-kappaB degradation to enhance NF-kappaB activity in prostate cancer cells. Mol Cancer Res, 2010a. 8(1): p. 119-30.
Zoubeidi, A., K. Chi, and M. Gleave, Targeting the cytoprotective chaperone, clusterin, for treatment of advanced cancer. Clin Cancer Res, 2010b. 16(4): p. 1088-93.
Zoubeidi, A., et al., Cooperative interactions between androgen receptor (AR) and heat-shock protein 27 facilitate AR transcriptional activity. Cancer Res, 2007. 67(21): p. 10455-65.
Zoubeidi, A., et al., Hsp27 promotes insulin-like growth factor-I survival signaling in prostate cancer via p90Rsk-dependent phosphorylation and inactivation of BAD. Cancer Res, 2010c. 70(6): p. 2307-17.

1. A method for treating a mammalian subject afflicted with prostate cancer comprising administering to the mammalian subject i) an oligonucleotide which reduces clusterin expression and ii) an androgen receptor antagonist having the structure 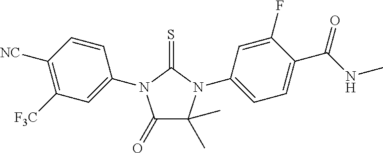 [SMILES: CNC(=O)c1ccc(N2C(=S)N(c3ccc(C#N)c(C(F)(F)F)c3)C(=O)C2(C)C)cc1F]
or a pharmaceutically acceptable salt thereof, each in an amount such that the combination is effective to treat the mammalian subject.

2. The method of claim 1, wherein the cancer is androgen-independent prostate cancer.

3. The method of claim 1, wherein the amount of the oligonucleotide and the amount of the androgen receptor antagonist or a pharmaceutically acceptable salt thereof when taken together is more effective to treat the subject than either agent administered alone.

4. The method of claim 1, wherein the amount of the oligonucleotide in is less than an amount which is clinically effective when the oligonucleotide is administered alone.

5. The method of claim 1, wherein the amount of the androgen receptor antagonist or a pharmaceutically acceptable salt thereof is less than an amount which is clinically effective when antagonist or salt thereof is administered alone.

6. The method of claim 1, wherein the amount of the oligonucleotide and the amount of the androgen receptor antagonist or a pharmaceutically acceptable salt thereof when taken together is effective to reduce a clinical symptom of prostate cancer in the subject.

7. (canceled)

8. The method of any one of claims 1-7, wherein the oligonucleotide is an antisense oligonucleotide.

9. The method of claim 8, wherein the antisense oligonucleotide spans either the translation initiation site or the translation termination site of clusterin-encoding mRNA.

10. The method of claim 9, wherein the antisense oligonucleotide comprises nucleotides, the sequence of which is set forth in one of SEQ ID NOS: 1 to 11.

11. The method of claim 9, wherein the antisense oligonucleotide comprises nucleotides, the sequence of which is set forth in SEQ ID NO: 3.

12. The method of claim 10, wherein the antisense oligonucleotide is modified to enhance in vivo stability relative to an unmodified oligonucleotide of the same sequence.

13. The method of claim 12, wherein the oligonucleotide is custirsen.

14. The method of claim 13, wherein the amount of custirsen is less than 640 mg.

15. (canceled)

16. The method of claim 13, wherein the amount of custirsen is administered intravenously once in a seven day period.

17. The method of claim 1, wherein the amount of the androgen receptor antagonist is less than 240 mg.

18-20. (canceled)

21. The method of claim 1, wherein the amount of the androgen receptor antagonist is administered orally once per day.

22. A method for treatment of a mammalian subject afflicted with androgen-independent prostate cancer, which comprises administering to the subject i) an oligonucleotide which reduces clusterin expression and ii) an androgen receptor antagonist, each in an amount such that the combination is effective to treat the mammalian subject.

23. The method of claim 22, wherein the androgen receptor antagonist is a non-steroidal anti-androgen.

24. The method of claim 22, wherein the androgen receptor antagonist is AR1.

25. The method of claim 1, wherein the combination of the oligonucleotide and the androgen receptor antagonist is effective to decrease androgen receptor translocation from the cytoplasm to the nucleus of the tumor cells; and/or
to increase the proteasome degradation of the androgen receptor protein in the tumor cells; and/or
to decrease androgen receptor transcriptional activity in the tumor cells; and/or
to decrease the amount of phosphorylated AFT in the tumor cells; and/or
to decrease the amount of phosphorylated ERK in the tumor cells; and/or
to inhibit the proliferation of prostate cancer cells.

26-30. (canceled)

31. A method of increasing the sensitivity of AR1 resistant prostate cancer cells to AR1 comprising treating the AR1 resistant prostate cancer cells with custirsen.

32. A pharmaceutical composition comprising an amount of an oligonucleotide which reduces clusterin expression and an androgen receptor antagonist.

33. (canceled)

34. A composition for treating a mammalian subject afflicted with prostate cancer comprising i) an oligonucleotide which reduces clusterin expression and ii) an androgen receptor antagonist having the structure 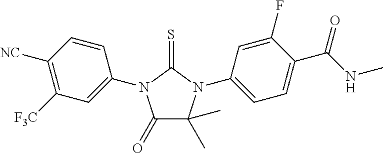 [SMILES: CNC(=O)c1ccc(N2C(=S)N(c3ccc(C#N)c(C(F)(F)F)c3)C(=O)C2(C)C)cc1F]
or a pharmaceutically acceptable salt thereof.


SEQUENCE LISTING (50 sequences)

SEQ ID NO: 1 - DNA, 21 bp, Homo sapiens
gcacagcaggagaatcttcat

SEQ ID NO: 2 - DNA, 21 bp, Homo sapiens
tggagtctttgcacgcctcgg

SEQ ID NO: 3 - DNA, 21 bp, Homo sapiens
cagcagcagagtcttcatcat

SEQ ID NO: 4 - DNA, 21 bp, Homo sapiens
attgtctgagaccgtctggtc

SEQ ID NO: 5 - DNA, 21 bp, Homo sapiens
ccttcagctttgtctctgatt

SEQ ID NO: 6 - DNA, 21 bp, Homo sapiens
agcagggagtcgatgcggtca

SEQ ID NO: 7 - DNA, 21 bp, Homo sapiens
atcaagctgcggacgatgcgg

SEQ ID NO: 8 - DNA, 21 bp, Homo sapiens
gcaggcagcccgtggagttgt

SEQ ID NO: 9 - DNA, 21 bp, Homo sapiens
ttcagctgctccagcaaggag

SEQ ID NO: 10 - DNA, 21 bp, Homo sapiens
aatttagggttcttcctggag

SEQ ID NO: 11 - DNA, 21 bp, Homo sapiens
gctgggcggagttgggggcct

SEQ ID NO: 12 - DNA, 17 bp, Homo sapiens
ggtgtagacgccgcacg

SEQ ID NO: 13 - DNA, 16 bp, Homo sapiens
gcagcgcagcccctgg

SEQ ID NO: 14 - DNA, 22 bp, Homo sapiens
gcagcagccgcagcccggctcc

SEQ ID NO: 15 - DNA, 18 bp, Homo sapiens
agccgcagcccggctcct

SEQ ID NO: 16 - DNA, 20 bp, Homo sapiens
cagcagccgcagcccggctc

SEQ ID NO: 17 - DNA, 20 bp, Homo sapiens
cagcagccgcagcccggctc

SEQ ID NO: 18 - DNA, 20 bp, Homo sapiens
agcagccgcagcccggctcc

SEQ ID NO: 19 - DNA, 21 bp, Artificial
  RNAi for human clusterin, the sequence is synthesized
ccagagcucgcccuucuactt

SEQ ID NO: 20 - DNA, 21 bp, Artificial
  RNAi for human clusterin, the sequence is synthesized
guagaagggcgagcucuggtt

SEQ ID NO: 21 - DNA, 21 bp, Artificial
  RNAi for human clusterin, the sequence is synthesized
gaugcucaacaccuccucctt

SEQ ID NO: 22 - DNA, 21 bp, Artificial
  RNAi for human clusterin, the sequence is synthesized
ggaggagguguugagcauctt

SEQ ID NO: 23 - DNA, 19 bp, Artificial
  RNAi for human clusterin, the sequence is synthesized
uaauucaacaaaacugutt

SEQ ID NO: 24 - DNA, 21 bp, Artificial
  RNAi for human clusterin, the sequence is synthesized
gacaguuuuauugaauuagtt

SEQ ID NO: 25 - DNA, 19 bp, Artificial
  RNAi for human clusterin, the sequence is synthesized
uaauucaacaaaacugutt

SEQ ID NO: 26 - DNA, 19 bp, Artificial
  RNAi for human clusterin, the sequence is synthesized
acaguuuuguugaauuatt

SEQ ID NO: 27 - DNA, 21 bp, Artificial
  RNAi for human clusterin, the sequence is synthesized
augaugaagacucugcugctt

SEQ ID NO: 28 - DNA, 21 bp, Artificial
  RNAi for human clusterin, the sequence is synthesized
gcagcagagucuucaucautt

SEQ ID NO: 29 - DNA, 22 bp, Artificial
  RNAi for human clusterin, the sequence is synthesized
ugaaugaagggacuaaccugtt

SEQ ID NO: 30 - DNA, 22 bp, Artificial
  RNAi for human clusterin, the sequence is synthesized
cagguuagucccuucauucatt

SEQ ID NO: 31 - DNA, 22 bp, Artificial
  RNAi for human clusterin, the sequence is synthesized
cagaaauagacaaaguggggtt

SEQ ID NO: 32 - DNA, 22 bp, Artificial
  RNAi for human clusterin, the sequence is synthesized
ccccacuuugucuauuucugtt

SEQ ID NO: 33 - DNA, 22 bp, Artificial
  RNAi for human clusterin, the sequence is synthesized
acagagacuaagggaccagatt

SEQ ID NO: 34 - DNA, 22 bp, Artificial
  RNAi for human clusterin, the sequence is synthesized
acagagacuaagggaccagatt

SEQ ID NO: 35 - DNA, 21 bp, Artificial
  RNAi for human clusterin, the sequence is synthesized
ccagagcucgcccuucuactt

SEQ ID NO: 36 - DNA, 21 bp, Artificial
  RNAi for human clusterin, the sequence is synthesized
guagaagggcgagcucuggtt

SEQ ID NO: 37 - DNA, 21 bp, Artificial
  RNAi for human clusterin, the sequence is synthesized
gucccgcaucguccgcagctt

SEQ ID NO: 38 - DNA, 21 bp, Artificial
  RNAi for human clusterin, the sequence is synthesized
gcugcggacgaugcgggactt

SEQ ID NO: 39 - DNA, 21 bp, Artificial
  RNAi for human clusterin, the sequence is synthesized
cuaauucaauaaaacuguctt

SEQ ID NO: 40 - DNA, 21 bp, Artificial
  RNAi for human clusterin, the sequence is synthesized
gacaguuuuauugaauuagtt

SEQ ID NO: 41 - DNA, 19 bp, Artificial
  RNAi for human clusterin, the sequence is synthesized
augaugaagacucugcugc

SEQ ID NO: 42 - DNA, 19 bp, Artificial
  RNAi for human clusterin, the sequence is synthesized
gcagcagagucuucaucau

SEQ ID NO: 43 - DNA, 2877 bp, Homo sapiens
gctttccgcggcattctttgggcgtgagtcatgcaggtttgcagccagccccaaagggggtgtgtgcgcgagcagagcgctataaatacggcgcctcccagtgcccacaacgcggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcacactgtttcaaaagttaaaaaaaaaaaaaaaaaa

SEQ ID NO: 44 - DNA, 21 bp, Artificial
  Control antisense oligonucleotide sequence, the sequence is synthesized
cagcgctgacaacagtttcat

SEQ ID NO: 45 - DNA, 20 bp, Artificial
  Target gene expression forward primer, the sequence is synthesized
taccagctcaccaagctcct

SEQ ID NO: 46 - DNA, 20 bp, Artificial
  Target gene expression reverse primer, the sequence is synthesized
gcttcactgggtgtggaaat

SEQ ID NO: 47 - DNA, 20 bp, Artificial
  Target gene expression forward primer, the sequence is synthesized
cacagcctgtttcatcctga

SEQ ID NO: 48 - DNA, 20 bp, Artificial
  Target gene expression reverse primer, the sequence is synthesized
aggtccatgaccttcacagc

SEQ ID NO: 49 - DNA, 20 bp, Artificial
  Target gene expression forward primer, the sequence is synthesized
aaatctggcaccacaccttc

SEQ ID NO: 50 - DNA, 20 bp, Artificial
  Target gene expression reverse primer, the sequence is synthesized
agcactgtgttggcgtacag